(19) United States
(12) Patent Application Publication
(10) Pub. No.: US 2017/0304658 A1
(43) Pub. Date: Oct. 26, 2017

(54) GEL/GEL COMPOSITION COMPRISING A UV-SCREENING AGENT

(71) Applicant: L'OREAL, PARIS (FR)

(72) Inventors: Angelina ROUDOT, LE KREMELIN BICETRE (FR); Anne FALIP, ALFORTVILLE (FR); Didier CANDAU, BIEVRES (FR); Elodie VALVERDE, PARIS (FR); Laure FAGEON, PARIS (FR)

(73) Assignee: L'OREAL, PARIS (FR)

(21) Appl. No.: 15/507,318

(22) PCT Filed: Aug. 26, 2015
(86) PCT No.: PCT/IB2015/056471

§ 371 Date: Feb. 28, 2017

(30) Foreign Application Priority Data
Aug. 28, 2014 (FR) 14 58031
Aug. 28, 2014 (FR) 14 58032
Aug. 28, 2014 (FR) 14 58034

(51) Int. Cl. A61Q 17/04; C08F 220/56; A61K 8/73; C08G 77/04; A61K 8/04

(52) U.S. Cl. CPC A61Q 17/04; C08G 77/04; A61K 8/042; A61K 8/73; C08F 220/56

(57) ABSTRACT

A composition, in particular a cosmetic composition, for making up and/or caring for keratin materials, including at least one aqueous phase gelled with at least one non-starchy hydrophilic gelling agent; at least one oily phase gelled with at least one non-cellulose-based lipophilic gelling agent other than apolar hydrocarbon-based waxes with a melting point of greater than 75.0° C. and silicone polyamides; the phases forming therein a macroscopically homogeneous mixture; the composition also including at least one UV-screening agent.

The present invention is directed toward proposing for the field of antisun protection and more particularly for the field of compositions for caring for and/or making up keratin materials, especially the skin and/or the lips, a novel presentation form that is most particularly advantageous with regard to its technical performance and especially in terms of photoprotection the sensations it affords the user during its application thereto, in particular to the skin.

The term “keratin materials” especially means the skin, the lips, and also keratin fibers such as the hair, in particular the skin and/or the hair, and preferably the skin.

It is known that radiation with wavelengths of between 280 nm and 400 nm makes possible tanning of the human epidermis and that radiation with wavelengths of between 280 and 320 nm, known under the name of UV-B rays, harms the development of a natural tan. Exposure is also liable to induce impairment of the biomechanical properties of the epidermis, which is reflected by the appearance of wrinkles, leading to premature aging of the skin.

It is also known that UV-A rays with wavelengths of between 320 and 400 nm penetrate more deeply into the skin than UV-B rays. UV-A rays cause immediate and persistent browning of the skin. Daily exposure to UVA rays, even for a short period, under normal conditions may lead to degradation of the collagen and elastin fibers, which is reflected by a change in the skin's microrelief, the appearance of wrinkles and non-uniform pigmentation (liver spots, or heterogeneity of the complexion).

Many photoprotective compositions have been proposed to date to overcome the effects induced by UVA and/or UVB radiation. They generally contain liposoluble and water-soluble organic UV-screening agents which function by absorbing UV rays depending on their intrinsic chemical nature.

It is known that the formulation of emulsions with high contents of screening agents, which are necessary for achieving high levels of screening efficacy, does not lend itself to quick and easy development of a varied range of compositions and textures.

Thus, in a given emulsified system, it often proves to be difficult to integrate UV-screening agents in high content, without impairing stability thereof. It is then necessary to readjust the formulation and in particular to reformulate the emulsified system.

It is moreover known that screening formulations have uncomfortable or even unpleasant sensory aspects masking the freshness and comfort of the formulations. The weak point of screening emulsions with a high protection factor is often a strong greasy and tacky feel, and thus a lack of lightness of the textures obtained, but also a shiny appearance on the skin which may be offputting, in particular for makeup compositions. It is thus difficult to reconcile in the same composition opposing technical performance qualities, such as a high level of UV protection, which entails a greasy and tacky finish on the skin, and a pleasant sensory aspect, attributed, inter alia, to the fresh sensation, and also a smooth, uniform look.

There thus remains a need for UV antisun protection compositions which are stable, efficient for photoprotection and which do not have the drawbacks presented above, in particular which afford an immediate visual result on the skin with a pleasant sensory aspect, especially a light, fresh sensation on application.

In the field of makeup, especially for foundations, compositions are also sought that allow homogeneous deposition, giving a visibly smoother appearance, and that are efficient in terms of photoprotection.

Unexpectedly and advantageously, the inventors have shown that this need can be met by means of the photoprotective compositions according to the present invention.

Thus, according to one of its aspects, the present invention is directed toward a composition, in particular a cosmetic composition for making up and/or caring for keratin materials, comprising:
at least one aqueous phase gelled with at least one non-starchy hydrophilic gelling agent,
at least one oily phase gelled with at least one non-cellulose-based lipophilic gelling agent other than apolar hydrocarbon-based waxes with a melting point of greater than 75.0° C., preferably greater than 80.0° C., and silicone polyamides,

said phases forming therein a macroscopically homogeneous mixture;

said composition also comprising at least one UV-screening agent.

Advantageously, the UV-screening agent(s) are totally or partly, and preferably solely, present in the gelled aqueous phase or are totally or partly, and preferably solely, present in the gelled oily phase.

According to a particular embodiment, the present invention is directed toward a composition, especially a cosmetic composition for making up and/or caring for keratin materials, comprising:
at least one aqueous phase gelled with at least one non-starchy hydrophilic gelling agent,
at least one oily phase gelled with at least one non-cellulose-based lipophilic gelling agent other than apolar hydrocarbon-based waxes and silicone polyamides,

said phases forming therein a macroscopically homogeneous mixture;

said composition also comprising at least one UV-screening agent.

Contrary to all expectation, and as emerges from the examples given below, the inventors have found that the formulation of a UV-screening agent in a gel-gel architecture as defined above makes it possible to obtain a composition that can provide freshness and lightness even when said composition contains a high content of UV-screening agents. In addition, the gel-gel architecture as defined above makes it possible to maintain the photoprotection properties and leads to stable compositions, especially without any appearance of phase separation.

Application of the composition to the skin gives a smooth, uniform deposit.

“Gel-gel” compositions have already been proposed in the cosmetic field. Formulations of this type combine a gelled aqueous phase with a gelled oily phase. Thus, gel/gel formulations are described in Almeida et al., Pharmaceutical Development and Technology, 2008, 13:487, tables 1 and 2, page 488; WO 99/65455; PI 0405758-9; WO 99/62497; JP 2005-112834 and WO 2008/081175. However, to the inventors' knowledge, this type of composition does not at the present time make it possible to ensure all the essential properties expected in the cosmetic field, such as a pleasant texture when taking up the product, a deposit with reduced tack, which is comfortable and uniform especially for making up, or stability of the formulation.

Patent application WO 2013/093869 also discloses compositions comprising at least:
a gelled aqueous phase containing at least 3% by weight, relative to its total weight, of particles of at least one carboxy(C₁-C₄)alkyl starch and whose particle size ranges from 25 to 300 μm, and
an oily phase gelled using at least one texturing agent,

with one of said phases constituting the continuous phase of said composition in which the other phase is uniformly dispersed.

As stated above, the inventors have found that the use of UV-screening agents in a multi-phase composition according to the invention makes it possible to ensure the persistence of the photoprotective properties and the stability.

Thus, a composition according to the invention shows very good stability both in terms of visual stability (no phase separation) and of photoprotective properties, while at the same time affording the user a light, fresh sensation on application.

Finally, the composition proves to be easy to apply to the surface of the intended keratin material and gives a smooth, uniform deposit in accordance with the requirements in the field of makeup.

According to another of its aspects, a subject of the invention is also a process for preparing a composition, in particular a cosmetic composition for making up and/or caring for keratin materials, comprising at least one step of mixing:
an aqueous phase gelled with at least one non-starchy hydrophilic gelling agent; and
at least one oily phase gelled with at least one non-cellulose-based lipophilic gelling agent other than apolar hydrocarbon-based waxes with a melting point of greater than 75.0° C., preferably greater than 80.0° C., and silicone polyamides;

said phases forming therein a macroscopically homogeneous mixture;

said composition also comprising at least one UV-screening agent.

According to one embodiment variant, this process may advantageously comprise a step of mixing at least two and better still at least three or more gelled phases.

More particularly, the invention is directed toward a process for preparing a composition, in particular a cosmetic composition for making up and/or caring for keratin materials, comprising at least one step of mixing:
an aqueous phase gelled with at least one non-starchy hydrophilic gelling agent; and
at least one oily phase gelled with at least one non-cellulose-based lipophilic gelling agent other than apolar hydrocarbon-based waxes and silicone polyamides;

said phases forming therein a macroscopically homogeneous mixture;

said composition also comprising at least one UV-screening agent.

For obvious reasons, the number of gelled aqueous phases and of gelled oily phases to be considered for forming a composition according to the invention may range for each of the two types of phase beyond two.

Advantageously, the mixing of the phases may be performed at room temperature.

However, the process of the invention may comprise, if necessary, a step of heating the mixture.

According to one embodiment variant, the final formulation may be manufactured without following a particular order of introduction of the various constituents and, in certain cases, a “one-pot” manufacture may be performed.

According to a particular embodiment, the representative gelled phases of the same type of architecture are gelled with a different gelling agent.

Multi-phase formulations may thus be developed.

According to another of its aspects, a subject of the invention is also a cosmetic process for making up and/or caring for keratin materials, in particular bodily and/or facial skin, and/or keratin fibers, especially the hair, comprising at least one step which consists in applying to said keratin material a composition according to the invention.

According to another of its aspects, a subject of the invention is also a cosmetic process for making up and/or caring for keratin materials, in particular bodily and/or facial skin, and/or keratin fibers, especially the hair, comprising at least the application to said keratin materials of a macroscopically homogeneous composition obtained by extemporaneous mixing, before application or at the time of application to said keratin material, of at least one aqueous phase gelled with at least one non-starchy hydrophilic gelling agent, and at least one oily phase gelled with at least one non-cellulose-based lipophilic gelling agent other than apolar hydrocarbon-based waxes with a melting point of greater than 75.0° C., preferably greater than 80.0° C., and silicone polyamides, and said composition also comprising at least one UV-screening agent.

According to another of its aspects, a subject of the invention is also a cosmetic process for limiting the darkening of the skin and/or improving the color and/or uniformity of the complexion, comprising the application, to the surface of the keratin material, of a composition according to the invention.

It also relates to a non-therapeutic cosmetic process for preventing and/or treating the signs of ageing of a keratin material, comprising the application, to the surface of the keratin material, of at least one composition according to the invention.

Definitions

For the purposes of the present invention, the term “UV-screening agent” means any organic compound (comprising at least carbon and hydrogen atoms) or any mineral compound (not comprising any carbon atoms) which is capable of screening out, by absorption and/or scattering and/or reflection, UV radiation ranging from 280 nm to 400 nm and which does not have sufficient covering power to produce a color on the surface of a keratin material by application of a composition comprising same.

For the purposes of the present invention, the term “wax” generally means a lipophilic compound that is solid at room temperature (25° C.), with a solid/liquid reversible change of state, having a melting point of greater than or equal to 30° C., which may be up to 200° C. and in particular up to 120° C.

For the purpose of the invention, the melting temperature or melting point corresponds to the temperature of the most endothermic peak observed on thermal analysis (DSC) as described in Standard ISO 11357-3; 1999. The melting point of the wax may be measured using a differential scanning calorimeter (DSC), for example the calorimeter sold under the name “MDSC 2920” by the company TA Instruments.

The measurement protocol is as follows:

A 5 mg sample of wax placed in a crucible is subjected to a first temperature rise from −20° C. to 100° C., at a heating rate of 10° C./minute, and then is cooled from 100° C. to −20° C. at a cooling rate of 10° C./minute and is finally subjected to a second temperature rise ranging from −20° C. to 100° C. at a heating rate of 5° C./minute. During the second temperature increase, the variation of the difference in power absorbed by the empty crucible and by the crucible containing the sample of wax is measured as a function of the temperature. The melting point of the compound is the temperature value corresponding to the top of the peak of the curve representing the variation in the difference in power absorbed as a function of the temperature.

The term “hydrocarbon-based wax” means a wax formed essentially from, or even constituted by, carbon and hydrogen atoms, and optionally oxygen and nitrogen atoms, and not containing any silicon or fluorine atoms.

For the purposes of the present invention, the term “apolar wax” means a wax whose solubility parameter at 25° C. as defined below, δₐ, is equal to 0 (J/cm³)^{1/2}.

The definition and calculation of the solubility parameters in the Hansen three-dimensional solubility space are described in the article by C. M. Hansen: The three-dimensional solubility parameters, J. Paint Technol., 39, 105 (1967).

According to this Hansen space:

δ_{D} characterizes the London dispersion forces derived from the formation of dipoles induced during molecular impacts;

δₚ characterizes the Debye interaction forces between permanent dipoles and also the Keesom interaction forces between induced dipoles and permanent dipoles;

δₕ characterizes the specific interaction forces (such as hydrogen bonding, acid/base, donor/acceptor, etc.); and

δₐ is determined by the equation: δₐ=(δₚ²+δₕ²)^{1/2}.

The parameters δₚ, δₕ, δ_{D} and δₐ are expressed as (J/cm³)^{1/2}.

Apolar waxes are in particular hydrocarbon-based waxes constituted solely of carbon and hydrogen atoms, and free of heteroatoms such as N, O, Si and P.

For the purposes of the invention, the term “polyamide” means a compound containing at least 2 amide repeating units, preferably at least 3 amide repeating units and better still 10 amide repeating units.

The term “silicone polyamide” means a polyamide comprising a polyorganosiloxane chain formed essentially from, or even constituted by, carbon and hydrogen atoms and silicon atoms and especially —SiO groups.

Composition

To begin with, it is important to note that a composition according to the invention is different from an emulsion.

An emulsion generally consists of an oily liquid phase and an aqueous liquid phase. It is a dispersion of droplets of one of the two liquid phases in the other. The size of the droplets forming the dispersed phase of the emulsion is typically about a micrometer (0.1 to 100 μm). Furthermore, an emulsion requires the presence of a surfactant or an emulsifier to ensure its stability.

In contrast, a composition according to the invention consists of a macroscopically homogeneous mixture of two immiscible gelled phases. These two phases both have a gel-type texture. This texture is especially reflected visually by a consistent and/or creamy appearance.

The term “macroscopically homogeneous mixture” means a mixture in which each of the gelled phases cannot be individualized by the naked eye. More precisely, in a composition according to the invention, the gelled aqueous phase and the gelled oily phase interpenetrate and thus form a stable, consistent product. This consistency is achieved by mixing interpenetrated macrodomains. These interpenetrated macrodomains are not measurable objects. Thus, by microscope, the composition according to the invention is very different from an emulsion. A composition according to the invention cannot, either, be characterized as having a “sense”, i.e. an O/W or W/O sense; in other words a continuous phase and a dispersed phase cannot be defined.

Thus, a composition according to the invention has a consistency of gel type. The stability of the composition may be ensured without the mandatory presence of a surfactant. Consequently, a cosmetic composition according to the invention does not necessarily require any surfactant or silicone emulsifier to ensure its stability.

A composition according to the invention differs from an emulsion according to at least one of the following tests: test performed using a dyestuff, “drop test” and dilution test.

Test Performed Using a Dyestuff

It is known practice from the prior art to observe the intrinsic nature of a mixture of aqueous and oily gels in a composition of gel-gel type, for example, by introducing a dyestuff either into the aqueous gelled phase or into the lipophilic gelled phase, before the formation of the composition of gel-gel type. During visual inspection, in a composition of gel-gel type, the dyestuff appears uniformly dispersed, even if the dye is present solely in the gelled aqueous phase or in the gelled oily phase. Specifically, if two different dyes of different colors are introduced, respectively, into the oily phase and into the aqueous phase, before formation of the composition of gel-gel type, the two colors may be observed as being uniformly dispersed throughout the composition of gel-gel type. This is different from an emulsion in which, if a dye, which is soluble in water or soluble in oil, is introduced, respectively, into the aqueous and oily phases, before forming the emulsion, the color of the dye present will only be observed in the outer phase (Remington: The Science and Practice of Pharmacy, 19th Edition (1995), Chapter 21, page 282).

Drop Test

It is also known practice to distinguish a composition of gel-gel type from an emulsion by performing a “drop test”. This test consists in demonstrating the bi-continuous nature of a composition of gel-gel type. Specifically, as mentioned previously, the consistency of a composition is obtained by means of the interpenetration of the aqueous and oily gelled domains. Consequently, the bi-continuous nature of a composition of gel-gel type may be demonstrated by means of a simple test with, respectively, hydrophilic and hydrophobic solvents. This test consists in depositing, firstly, one drop of a hydrophilic solvent on a first sample of the test composition, and, secondly, one drop of a hydrophobic solvent on a second sample of the same test composition, and in analyzing the behavior of the two drops of solvents. In the case of an O/W emulsion, the drop of hydrophilic solvent diffuses into the sample and the drop of hydrophobic solvent remains at the surface of the sample. In the case of a W/O emulsion, the drop of hydrophilic solvent remains at the surface of the sample and the drop of hydrophobic solvent diffuses throughout the sample. Finally, in the case of a composition of gel-gel type (bi-continuous system), the hydrophilic and hydrophobic drops diffuse throughout the sample.

Dilution Test

In the case of the present invention, the test that will be preferred for distinguishing a composition of gel-gel type from an emulsion is a dilution test. Specifically, in a composition of gel-gel type, the aqueous and oily gelled domains interpenetrate and form a consistent and stable composition, in which the behavior in water and in oil is different from the behavior of an emulsion. Consequently, the behavior during dilution of a composition of gel-gel type (bi-continuous system) may be compared to that of an emulsion and will obviously lead to different results.

More specifically, the dilution test consists in placing 40 g of product and 160 g of dilution solvent (water or oil) in a beaker. The dilution is performed with controlled stirring to avoid any emulsification. In particular, this is performed using a planetary mixer: Speed Mixer™ DAC400FVZ. The speed of the mixer is set at 1500 rpm for 4 minutes. Finally, observation of the resulting sample is performed using a light microscope at a magnification of ×100 (×10×10). It may be noted that oils such as Parleam® and Xiameter PMX-200 Silicone Fluid 5CS® sold by Dow Corning are suitable as dilution solvent, in the same respect as one of the oils contained in the composition.

In the case of a composition of gel-gel type (bi-continuous system), when it is diluted in oil or in water, a heterogeneous appearance is always observed. When a composition of gel-gel type (bi-continuous system) is diluted in water, pieces of oily gel in suspension are observed, and when a composition of gel-gel type (bi-continuous system) is diluted in oil, pieces of aqueous gel in suspension are observed.

In contrast, during dilution, emulsions have a different behavior. When an O/W emulsion is diluted in an aqueous solvent, it gradually reduces without having a heterogeneous and lumpy appearance. This same O/W emulsion, on dilution with oil, has a heterogeneous appearance (pieces of O/W emulsion suspended in the oil). When a W/O emulsion is diluted with an aqueous solvent, it has a heterogeneous appearance (pieces of W/O emulsion suspended in the water). This same W/O emulsion, when diluted in oil, gradually reduces without having a heterogeneous and lumpy appearance.

According to the present invention, the aqueous gelled phase and the oily gelled phase forming a composition according to the invention are present therein in a weight ratio ranging from 95/5 to 5/95. More preferentially, the aqueous phase and the oily phase are present in a weight ratio ranging from 20/80 to 80/20 and even more preferentially ranging from 30/70 to 70/30.

The ratio between the two gelled phases is adjusted according to the desired cosmetic properties.

Thus, in the case of a care or makeup composition, in particular for the face, it will be advantageous to promote an aqueous gelled phase/oily gelled phase weight ratio of greater than or equal to 1, especially ranging from 50/50 to 90/10, preferably ranging from 60/40 to 80/20. Advantageously, a composition according to the invention may thus be in the form of a creamy gel having a minimum stress below which it does not flow unless it has been subjected to an external mechanical stress.

As emerges from the text hereinbelow, a composition according to the invention may have a minimum threshold stress of 1.5 Pa and in particular greater than 10 Pa. The composition according to the invention may have a threshold stress pf less than 10 000 Pa, preferably less than 5000 Pa.

It may also advantageously have a stiffness modulus G* at least equal to 400 Pa and preferably greater than 1000 Pa. The composition according to the invention may have a stiffness modulus G* preferably less than 50 000 Pa, preferably less than 5000 Pa.

The viscosity of the hydrophilic phase to the viscosity of the lipophilic phase (measured at 25° C. and 100 s⁻¹) preferably ranges from 0.2 to 3, preferably 0.5 to 1.5.

According to an advantageous embodiment variant, the gelled phases under consideration to form a composition according to the invention have, respectively, a threshold stress of greater than 1.5 Pa and preferably greater than 10 Pa.

The gelled phases under consideration to form a composition according to the invention may have a threshold stress of less than 10 000 Pa and preferably less than 5000 Pa.

Characterization of the threshold stresses is performed by oscillating rheology measurements. Methodology is proposed in the illustrative chapter of the present text.

In general, the corresponding measurements are taken at 25° C. using a Haake RS600 imposed-stress rheometer equipped with a plate-plate measuring body (60 mm diameter) fitted with an anti-evaporation device (bell jar). For each measurement, the sample is placed delicately in position and the measurements start 5 minutes after placing the sample in the jaws (2 mm). The test composition is then subjected to a stress ramp from 10⁻² to 10³ Pa at a set frequency of 1 Hz.

A composition according to the invention may also have a certain elasticity. This elasticity may be characterized by a stiffness modulus G* which, under this minimum stress threshold, may be at least equal to 400 Pa and preferably greater than 1000 Pa. The value G* of a composition may be obtained by subjecting the composition under consideration to a stress ramp from 10⁻² to 10³ Pa at a set frequency of 1 Hz.

Hydrophilic Gelling Agent

For the purposes of the present invention, the term “hydrophilic gelling agent” means a compound that is capable of gelling the aqueous phase of the compositions according to the invention.

For the purposes of the present invention, the term “non-starchy hydrophilic gelling agent” means a hydrophilic gelling agent which is different from a starch.

The hydrophilic gelling agent is thus present in the aqueous phase of the composition.

The gelling agent may be water-soluble or water-dispersible.

The hydrophilic gelling agents are preferably non-emulsifying; preferably, they do not contain any fatty chains such as alkyl chains greater than C₇ and especially ranging from C₇ to C₂₄.

As stated above, the aqueous phase of a composition according to the invention is gelled with at least one hydrophilic gelling agent.

The non-starchy hydrophilic gelling agent may be chosen from synthetic polymeric gelling agents, mixed silicates and fumed silicas, non-starchy polymeric gelling agents which are natural or of natural origin, especially non-starchy polysaccharides, and mixtures thereof.

Preferably, the hydrophilic gelling agent is chosen from synthetic polymeric gelling agents.

I. Non-Starchy Polymeric Gelling Agents that are Natural or of Natural Origin

The polymeric hydrophilic gelling agents that are suitable for use in the invention may be natural or of natural origin.

For the purposes of the invention, the term “of natural origin” is intended to denote polymeric gelling agents obtained by modification of natural polymeric gelling agents.

These gelling agents may be particulate or non-particulate.

More specifically, these gelling agents fall within the category of polysaccharides.

Non-Starchy Polysaccharides

In general, the non-starchy polysaccharides may be chosen from polysaccharides produced by microorganisms; polysaccharides isolated from algae, and higher plant polysaccharides, such as homogeneous polysaccharides, in particular celluloses and derivatives thereof or fructosans, heterogeneous polysaccharides such as gum arabics, galactomannans, glucomannans and pectins, and derivatives thereof; and mixtures thereof.

In particular, the polysaccharides may be chosen from fructans, gellans, glucans, glycogen, pullulan, dextrans, celluloses and derivatives thereof, in particular methylcelluloses, hydroxyalkylcelluloses, ethylhydroxyethylcelluloses and carboxymethylcelluloses, mannans, xylans, lignins, arabans, galactans, galacturonans, alginate-based compounds, chitin, chitosans, glucuronoxylans, arabinoxylans, xyloglucans, glucomannans, pectic acids and pectins, arabinogalactans, carrageenans, agars, glycosaminoglucans, gum arabics, tragacanth gums, ghatti gums, karaya gums, locust bean gums, galactomannans such as guar gums and nonionic derivatives thereof, in particular hydroxypropyl guar, and ionic derivatives thereof, biopolysaccharide gums of microbial origin, in particular scleroglucan or xanthan gums, mucopolysaccharides, and in particular chondroitin sulfates, and mixtures thereof.

These polysaccharides may be chemically modified, especially with urea or urethane groups or by hydrolysis, oxidation, esterification, etherification, sulfatation, phosphatation, amination, amidation or C₁-C₆ alkylation reaction, or by several of these modifications.

The derivatives obtained may be anionic, cationic, amphoteric or nonionic.

Advantageously, the polysaccharides may be chosen from carrageenans, in particular kappa carrageenan, gellan gum, agar-agar, xanthan gum, alginates-based compounds, in particular sodium alginate, scleroglucan gum, guar gum, inulin and pullulan, and mixtures thereof.

In general, the compounds of this type that may be used in the present invention are chosen from those described especially in Kirk-Othmer's Encyclopedia of Chemical Technology, Third Edition, 1982, volume 3, pp. 896-900, and volume 15, pp. 439-458, in Polymers in Nature by E. A. MacGregor and C. T. Greenwood, published by John Wiley & Sons, Chapter 6, pp. 240-328, 1980, in the book by Robert L. Davidson entitled Handbook of Water-Soluble Gums and Resins published by Mc Graw Hill Book Company (1980) and in Industrial Gums—Polysaccharides and their Derivatives, edited by Roy L. Whistler, Second Edition, published by Academic Press Inc.

Such a gelling agent may be used in a proportion of from 0.1% to 8% by weight of solids relative to the total weight of the aqueous phase, especially from 0.1% to 6% by weight and preferably between 0.5% and 2.5% by weight relative to the total weight of the aqueous phase.

More precisely, these polysaccharides that are suitable for use in the invention may be distinguished according to whether they are derived from microorganisms, from algae or from higher plants, and are detailed below.

Polysaccharides Produced by Microorganisms

Xanthan

Xanthan is a heteropolysaccharide produced at the industrial scale by the aerobic fermentation of the bacterium Xanthomonas campestris. Its structure consists of a main chain of β(1,4)-linked β-D-glucoses, similar to cellulose. One glucose molecule in two bears a trisaccharide side chain composed of an α-D-mannose, a β-D-glucuronic acid and a terminal β-D-mannose. The internal mannose residue is generally acetylated on carbon 6. About 30% of the terminal mannose residues bear a pyruvate group linked in chelated form between carbons 4 and 6. The charged pyruvic acids and glucuronic acids are ionizable, and are thus responsible for the anionic nature of xanthan (negative charge down to a pH equal to 1). The content of pyruvate and acetate residues varies according to the bacterial strain, the fermentation process, the conditions after fermentation and the purification steps. These groups may be neutralized in commercial products with Na⁺, K⁺ or Ca²⁺ ions (Satia company, 1986). The neutralized form may be converted into the acid form by ion exchange or by dialysis of an acidic solution.

Xanthan gums have a molecular weight of between 1 000 000 and 50 000 000 and a viscosity of between 0.6 and 1.65 Pa·s for an aqueous composition containing 1% of xanthan gum (measured at 25° C. on a Brookfield viscometer of LVT type at 60 rpm).

Xanthan gums are represented, for example, by the products sold under the names Rhodicare by the company Rhodia Chimie, under the name Satiaxane™ by the company Cargill Texturizing Solutions (for the food, cosmetic and pharmaceutical industries), under the name Novaxan™ by the company ADM, and under the names Kelzan® and Keltrol® by the company CP-Kelco.

Pullulan

Pullulan is a polysaccharide consisting of maltotriose units, known under the name α(1,4)-α(1,6)-glucan. Three glucose units in maltotriose are connected via an α(1,4) glycoside bond, whereas the consecutive maltotriose units are connected to each other via an α(1,6) glycoside bond.

Pullulan is produced, for example, under the reference Pullulan PF 20 by the group Hayashibara in Japan.

Dextran and Dextran Sulfate

Dextran is a neutral polysaccharide not bearing any charged groups, which is biologically inert, prepared by fermentation of beet sugar containing solely hydroxyl groups.

It is possible to obtain dextran fractions of different molecular weights from native dextran by hydrolysis and purification. Dextran may in particular be in the form of dextran sulfate.

Dextran is represented, for example, by the products sold under the name Dextran or Dextran T by the company Pharmacosmos, or under the name Dextran 40 Powder or Dextran 70 Powder by the company Meito Sangyo Co. Dextran sulfate is sold by the company PK Chemical A/S under the name Dextran sulfate.

Succinoglycan

Succinoglycan is an extracellular polymer of high molecular weight produced by bacterial fermentation, consisting of octasaccharide repeating units (repetition of 8 sugars). Succinoglycans are sold, for example, under the name Rheozan by the company Rhodia.

Scleroglucan

Scleroglucan is a nonionic branched homopolysaccharide consisting of β-D-glucan units. The molecules consist of a linear main chain formed from D-glucose units linked via β(1,3) bonds and of which one in three is linked to a side D-glucose unit via a β(1,6) bond.

A more complete description of scleroglucans and of their preparation may be found in U.S. Pat. No. 3,301,848.

Scleroglucan is sold, for example, under the name Amigel by the company Alban Müller, or under the name Actigum™ CS by the company Cargill.

Gellan Gum

Gellan gum is an anionic linear heteropolyoside based on oligoside units composed of 4 saccharides (tetra-oside). D-Glucose, L-rhamnose and D-glucuronic acid in 2:1:1 proportions are present in gellan gum in the form of monomer elements.

It is sold, for example, under the name Kelcogel CG LA by the company CP Kelco.

Polysaccharides Isolated from Algae

Galactans

The polysaccharide according to the invention may be a galactan chosen especially from agar and carrageenans.

Carrageenans are anionic polysaccharides constituting the cell walls of various red algae (Rhodophyceae) belonging to the Gigartinacae, Hypneaceae, Furcellariaceae and Polyideaceae families. They are generally obtained by hot aqueous extraction from natural strains of said algae. These linear polymers, formed by disaccharide units, are composed of two D-galactopyranose units linked alternately by α(1,3) and β(1,4) bonds. They are highly sulfated polysaccharides (20-50%) and the α-D-galactopyranosyl residues may be in 3,6-anhydro form. Depending on the number and position of sulfate-ester groups on the repeating disaccharide of the molecule, several types of carrageenans are distinguished, namely: kappa-carrageenans, which bear one sulfate-ester group, iota-carrageenans, which bear two sulfate-ester groups, and lambda-carrageenans, which bear three sulfate-ester groups.

Carrageenans are composed essentially of potassium, sodium, magnesium, triethanolamine and/or calcium salts of polysaccharide sulfate esters.

Carrageenans are sold especially by the company SEPPIC under the name Solagum®, by the company Gelymar under the names Carragel®, Carralact® and Carrasol®, by the company Cargill, under the names Satiagel™ and Satiagum™, and by the company CP-Kelco under the names Genulacta®, Genugel® and Genuvisco®.

Galactans of agar type are galactose polysaccharides contained in the cell wall of some of these species of red algae (rhodophyceae). They are formed from a polymer group whose base backbone is a β(1,3) D-galactopyranose and α(1,4) L 3-6 anhydrogalactose chain, these units repeating regularly and alternately. The differences within the agar family are due to the presence or absence of sulfated methyl or carboxyethyl groups. These hybrid structures are generally present in variable percentage, depending on the species of algae and the harvest season.

Agar-agar is a mixture of polysaccharides (agarose and agaropectin) of high molecular mass, between 40 000 and 300 000 g.mol⁻¹. It is obtained by manufacturing algal extraction liquors, generally by autoclaving, and by treating these liquors which comprise about 2% of agar-agar, so as to extract the latter.

Agar is produced, for example, by the group B&V Agar Producers under the names Gold Agar, Agarite and Grand Agar by the company Hispanagar, and under the names Agar-Agar, QSA (Quick Soluble Agar), and Puragar by the company Setexam.

Furcellaran

Furcellaran is obtained commercially from red algae Furcellaria fasztigiata. Furcellaran is produced, for example, by the company Est-Agar.

Alginate-Based Compound

For the purposes of the invention, the term “alginate-based compound” means alginic acid, alginic acid derivatives and salts of alginic acid (alginates) or of said derivatives.

Preferably, the alginate-based compound is water-soluble.

Alginic acid, a natural substance resulting from brown algae or certain bacteria, is a polyuronic acid composed of 2 uronic acids linked by 1,4-glycosidic bonds: β-D-manuronic (M) acid and α-L-glucuronic (G) acid.

Alginic acid is capable of forming water-soluble salts (alginates) with alkali metals such as sodium, potassium or lithium, substituted cations of lower amines and of ammonium such as methylamine, ethanolamine, diethanolamine or triethanolamine. These alginates are water-soluble in aqueous medium at a pH equal to 4, but dissociate into alginic acid at a pH below 4.

This (these) alginate-based compound(s) are capable of crosslinking in the presence of at least one crosslinking agent, by formation of ionic bonds between said alginate-based compound(s) and said crosslinking agent(s). The formation of multiple crosslinking between several molecules of said alginate-based compound(s) leads to the formation of a water-insoluble gel.

Use is preferably made of alginate-based compounds with a weight-average molecular mass ranging from 10 000 to 1 000 000, preferably from 15 000 to 500 000 and better still from 20 000 to 250 000.

According to a preferred embodiment, the alginate-based compound is alginic acid and/or a salt thereof.

Advantageously, the alginate-based compound is an alginate salt, and preferably sodium alginate.

The alginate-based compound may be chemically modified, especially with urea or urethane groups or by hydrolysis, oxidation, esterification, etherification, sulfatation, phosphatation, amination, amidation or alkylation reaction, or by several of these modifications.

The derivatives obtained may be anionic, cationic, amphoteric or nonionic.

The alginate-based compounds that are suitable for use in the invention may be represented, for example, by the products sold under the names Kelcosol, Satialgine™, Cecalgum™ or Algogel™ by the company Cargill Products, under the name Protanal™ by the company FMC Biopolymer, under the name Grindsted® Alginate by the company Danisco, under the name Kimica Algin by the company Kimica, and under the names Manucol® and Manugel® by the company ISP.

Polysaccharides of Higher Plants

This category of polysaccharides may be divided into homogeneous polysaccharides (only one saccharide species) and heterogeneous polysaccharides composed of several types of saccharides.

a) Homogeneous Polysaccharides and Derivatives Thereof

The polysaccharide according to the invention may be chosen from celluloses and derivatives or fructosans.

Cellulose and Derivatives

The polysaccharide according to the invention may also be a cellulose or a derivative thereof, especially cellulose ethers or esters (e.g.: methylcellulose, carboxymethylcellulose, hydroxymethylcellulose, hydroxyethylcellulose, hydroxypropylcellulose, hydroxymethylpropylcellulose, cellulose acetate, cellulose nitrate, nitrocellulose).

According to the invention, the term “cellulose-based compound” means any polysaccharide compound bearing in its structure linear sequences of anhydroglucopyranose residues (AGU) linked together via β(1,4) bonds. The repeating unit is the cellobiose dimer. The AGUs are in chair conformation and bear 3 hydroxyl functions: 2 secondary alcohols (in position 2 and 3) and a primary alcohol (in position 6). The polymers thus formed combine together via intermolecular bonds of hydrogen bond type, thus giving the cellulose a fibrillar structure (about 1500 molecules per fiber).

The degree of polymerization differs enormously depending on the origin of the cellulose; its value may range from a few hundred to several tens of thousands.

Cellulose has the following chemical structure:

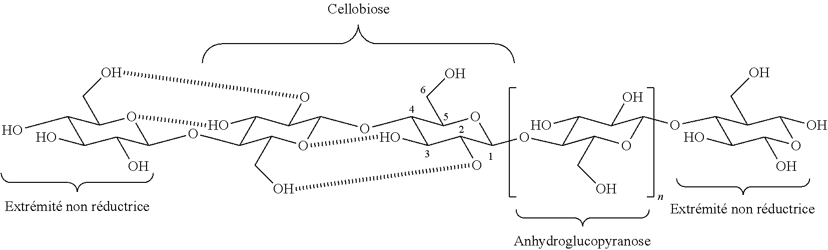

The hydroxyl groups of cellulose may react partially or totally with various chemical reagents to give cellulose derivatives having intrinsic properties. The cellulose derivatives may be anionic, cationic, amphoteric or nonionic. Among these derivatives, cellulose ethers, cellulose esters and cellulose ester ethers are distinguished.

Among the nonionic cellulose ethers, mention may be made of alkylcelluloses such as methylcelluloses and ethylcelluloses; hydroxyalkylcelluloses such as hydroxymethylcelluloses, hydroxyethylcelluloses and hydroxypropylcelluloses; and mixed hydroxy-alkylalkylcelluloses such as hydroxypropylmethylcelluloses, hydroxyethylmethylcelluloses, hydroxyethylethylcelluloses and hydroxybutylmethylcelluloses.

Among the anionic cellulose ethers, mention may be made of carboxyalkyl celluloses and salts thereof. By way of example, mention may be made of carboxymethylcelluloses, carboxymethylmethyl-celluloses and carboxymethylhydroxyethylcelluloses and sodium salts thereof.

Among the cationic cellulose ethers, mention may be made of crosslinked or non-crosslinked, quaternized hydroxyethylcelluloses.

The quaternizing agent may especially be glycidyltrimethylammonium chloride. Another cationic cellulose ether that may be mentioned is hydroxyethylcellulosehydroxypropyltrimethylammonium.

Among the cellulose esters are mineral esters of cellulose (cellulose nitrates, sulfates, phosphates, etc.), organic cellulose esters (cellulose monoacetates, triacetates, amidopropionates, acetatebutyrates, acetatepropionates and acetatetrimellitates, etc.), and mixed organic/mineral esters of cellulose, such as cellulose acetatebutyrate sulfates and cellulose acetatepropionate sulfates. Among the cellulose ester ethers, mention may be made of hydroxypropylmethylcellulose phthalates and ethylcellulose sulfates.

The cellulose-based compounds of the invention may be chosen from unsubstituted celluloses and substituted celluloses.

The celluloses and derivatives are represented, for example, by the products sold under the names Avicel® (microcrystalline cellulose, MCC) by the company FMC Biopolymers, under the name Cekol (carboxymethylcellulose) by the company Noviant (CP-Kelco), under the name Akucell AF (sodium carboxymethylcellulose) by the company Akzo Nobel, under the name Methocel™ (cellulose ethers), and under the names Aqualon® (carboxymethylcellulose and sodium carboxymethyl-cellulose), Benecel® (methylcellulose), Blanose™ (carboxymethylcellulose), Culminal® (methylcellulose, hydroxypropylmethylcellulose), Klucel® (hydroxypropylcellulose) and Natrosol® CS (hydroxyethylcellulose) by the company Hercules Aqualon.

Fructosans

The polysaccharide according to the invention may especially be a fructosan chosen from inulin and derivatives thereof (especially dicarboxy and carboxymethyl inulins).

Fructans or fructosans are oligosaccharides or polysaccharides comprising a sequence of anhydrofructose units optionally combined with several saccharide residues other than fructose. Fructans may be linear or branched. Fructans may be products obtained directly from a plant or microbial source or alternatively products whose chain length has been modified (increased or decreased) by fractionation, synthesis or hydrolysis, in particular enzymatic. Fructans generally have a degree of polymerization from 2 to about 1000 and preferably from 2 to about 60.

Three groups of fructans are distinguished. The first group corresponds to products whose fructose units are for the most part linked via β(2,1) bonds. These are essentially linear fructans such as inulins.

The second group also corresponds to linear fructoses, but the fructose units are essentially linked via β(2,6) bonds. These products are levans.

The third group corresponds to mixed fructans, i.e. containing β(2,6) and β(2,1) sequences. These are essentially branched fructans, such as graminans.

The fructans used in the compositions according to the invention are inulins. Inulin may be obtained, for example, from chicory, dahlia or Jerusalem artichoke, preferably from chicory.

In particular, the polysaccharide, especially the inulin, has a degree of polymerization from 2 to about 1000 and preferably from 2 to about 60, and a degree of substitution of less than 2 on the basis of one fructose unit.

The inulin used for this invention is represented, for example, by the products sold under the name Beneo™ inulin by the company Orafti, and under the name Frutafit® by the company Sensus.

b) Heterogeneous Polysaccharides and Derivatives Thereof

The polysaccharides that may be used according to the invention may be gums, for instance cassia gum, karaya gum, konjac gum, gum tragacanth, tara gum, acacia gum or gum arabic.

Gum Arabic

Gum arabic is a highly branched acidic polysaccharide which is in the form of mixtures of potassium, magnesium and calcium salts. The monomer elements of the free acid (arabic acid) are D-galactose, L-arabinose, L-rhamnose and D-glucuronic acid.

Galactomannans (guar, locust bean, fenugreek, tara gum) and derivatives (guar phosphate, hydroxypropyl guar, etc.)

Galactomannans are nonionic polyosides extracted from the endosperm of leguminous seeds, of which they constitute the storage carbohydrate.

Galactomannans are macromolecules consisting of a main chain of β(1,4) linked D-mannopyranose units, bearing side branches consisting of a single D-galactopyranose unit α(1,6) linked to the main chain. The various galactomannans differ, firstly, by the proportion of α-D-galactopyranose units present in the polymer, and secondly by significant differences in terms of distribution of galactose units along the mannose chain.

The mannose/galactose (M/G) ratio is about 2 for guar gum, 3 for tara gum and 4 for locust bean gum.

Galactomannans have the following chemical structure:

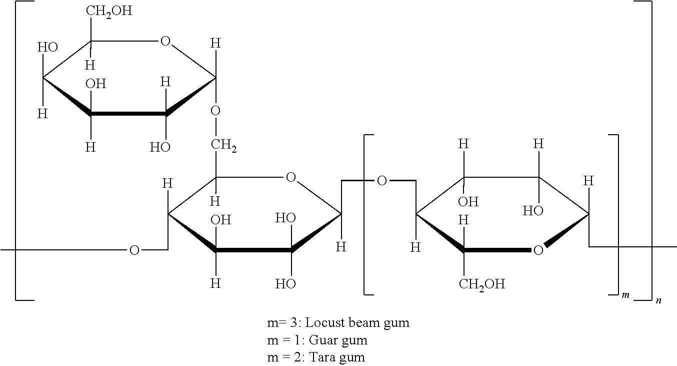

Guar

Guar gum is characterized by a mannose/galactose ratio of the order of 2/1. The galactose group is regularly distributed along the mannose chain.

The guar gums that may be used according to the invention may be nonionic, cationic or anionic. According to the invention, use may be made of chemically modified or unmodified nonionic guar gums.

The unmodified nonionic guar gums are, for example, the products sold under the names Vidogum GH, Vidogum G and Vidocrem by the company Unipektin and under the name Jaguar by the company Rhodia, under the name Meypro® Guar by the company Danisco, under the name Viscogum™ by the company Cargill, and under the name Supercol® guar gum by the company Aqualon.

The hydrolyzed nonionic guar gums that may be used according to the invention are represented, for example, by the products sold under the name Meyprodor® by the company Danisco.

The modified nonionic guar gums that may be used according to the invention are preferably modified with C₁-C₆ hydroxyalkyl groups, among which mention may be made, for example, of hydroxymethyl, hydroxyethyl, hydroxypropyl and hydroxybutyl groups.

Such nonionic guar gums optionally modified with hydroxyalkyl groups are sold, for example, under the trade names Jaguar HP60, Jaguar HP 105 and Jaguar HP 120 (hydroxypropyl guar) by the company Rhodia or under the name N-Hance® HP (hydroxypropyl guar) by the company Aqualon.

The cationic galactomannan gums preferably have a cationic charge density of less than or equal to 1.5 meq./g, more particularly between 0.1 and 1 meq./g. The charge density may be determined by the Kjeldahl method. It generally corresponds to a pH of the order of 3 to 9.

In general, for the purposes of the present invention, the term “cationic galactomannan gum” means any galactomannan gum containing cationic groups and/or groups that can be ionized into cationic groups.

The preferred cationic groups are chosen from those comprising primary, secondary, tertiary and/or quaternary amine groups.

The cationic galactomannan gums used generally have a weight-average molecular mass of between 500 and 5×10⁶ approximately and preferably between 10³ and 3×10⁶ approximately.

The cationic galactomannan gums that may be used according to the present invention are, for example, gums comprising tri(C₁-C₄)alkylammonium cationic groups. Preferably, 2% to 30% by number of the hydroxyl functions of these gums bear trialkylammonium cationic groups.

Mention may very particularly be made, among these trialkylammonium groups, of the trimethylammonium and triethylammonium groups.

Even more preferentially, these groups represent from 5% to 20% by weight relative to the total weight of the modified galactomannan gum.

According to the invention, the cationic galactomannan gum is preferably a guar gum comprising hydroxypropyltrimethylammonium groups, i.e. a guar gum modified, for example, with 2,3-epoxypropyltrimethylammonium chloride.

These galactomannan gums, in particular guar gums modified with cationic groups are products already known per se and are, for example, described in patents U.S. Pat. No. 3,589,578 and U.S. Pat. No. 4,031,307. Such products are moreover sold especially under the trade names Jaguar EXCEL, Jaguar C13 S, Jaguar C 15, Jaguar C 17 and Jaguar C162 (Guar Hydroxypropyltrimonium Chloride) by the company Rhodia, under the name Amilan® Guar (Guar Hydroxypropyltrimonium Chloride) by the company Degussa, and under the name N-Hance® 3000 (Guar Hydroxypropyltrimonium Chloride) by the company Aqualon.

The anionic guar gums that may be used according to the invention are polymers comprising groups derived from carboxylic, sulfonic, sulfenic, phosphoric, phosphonic or pyruvic acid. The anionic group is preferably a carboxylic acid group. The anionic group may also be in the form of an acid salt, especially a sodium, calcium, lithium or potassium salt.

The anionic guar gums that may be used according to the invention are preferentially carboxymethyl guar derivatives (carboxymethyl guar or carboxymethyl hydroxypropyl guar).

Locust Bean

Locust bean gum is extracted from the seeds of the locust bean tree (Ceratonia siliqua).

The unmodified locust bean gum that may be used in this invention is sold, for example, under the name Viscogum™ by the company Cargill, under the name Vidogum L by the company Unipektin and under the name Grinsted® LBG by the company Danisco.

The chemically modified locust bean gums that may be used in this invention may be represented, for example, by the cationic locust beans sold under the name Catinal CLB (locust bean hydroxypropyltrimonium chloride) by the company Toho.

Tara Gum

The tara gum that may be used in the context of this invention is sold, for example, under the name Vidogum SP by the company Unipektin.

Glucomannans (Konjac Gum)

Glucomannan is a polysaccharide of high molecular weight (500 000<Mglucomannan<2 000 000) composed of D-mannose and D-glucose units with a branch every 50 or 60 units approximately. It is found in wood, but is also the main constituent of konjac gum. Konjac (Amorphophallus konjac) is a plant of the Araceae family.

The products that may be used according to the invention are sold, for example, under the names Propol® and Rheolex® by the company Shimizu.

LM and HM Pectins, and Derivatives

Pectins are linear polymers of α-D-galacturonic acid (at least 65%) linked in positions 1 and 4 with a certain proportion of carboxylic groups esterified with a methanol group. About 20% of the sugars constituting the pectin molecule are neutral sugars (L-rhamnose, D-glucose, D-galactose, L-arabinose, D-xylose). L-Rhamnose residues are found in all pectins, incorporated into the main chain in positions 1,2.

Uronic acid molecules bear carboxyl functions. This function gives pectins the capacity for exchanging ions, when they are in COO⁻ form. Divalent ions (in particular calcium) have the capacity of forming ionic bridges between two carboxyl groups of two different pectin molecules.

In the natural state, a certain proportion of the carboxylic groups are esterified with a methanol group. The natural degree of esterification of a pectin may range between 70% (apple, lemon) and 10% (strawberry) depending on the source used. Using pectins with a high degree of esterification it is possible to hydrolyze the —COOCH₃ groups, so as to obtain weakly esterified pectins. Depending on the proportion of methylated or non-methylated monomers, the chain is thus more or less acidic. HM (high-methoxy) pectins are thus defined as having a degree of esterification of greater than 50%, and LM (low-methoxy) pectins are defined as having a degree of esterification of less than 50%.

In the case of amidated pectins, the —OCH₃ group is substituted with an —NH₂ group.

Pectins are especially sold by the company Cargill under the name Unipectine™, by the company CP-Kelco under the name Genu, and by Danisco under the name Grinsted Pectin.

Other Polysaccharides

Among the other polysaccharides that may be used according to the invention, mention may also be made of chitin (poly-N-acetyl-D-glucosamine, β(1,4)-2-acetamido-2-deoxy-D-glucose), chitosan and derivatives (chitosan-beta-glycerophosphate, carboxymethylchitin, etc.) such as those sold by the company France-Chitine; glycosaminoglycans (GAG) such as hyaluronic acid, chondroitin sulfate, dermatan sulfate, keratan sulfate, and preferably hyaluronic acid; xylans (or arabinoxylans) and derivatives.

Arabinoxylans are polymers of xylose and arabinose, all grouped under the name pentosans.

Xylans consist of a main chain of β(1,4) linked D-xylose units and on which are found three substituents (Rouau & Thibault, 1987): acid units, α-L-arabinofuranose units, side chains which may contain arabinose, xylose, galactose and glucuronic acid.

According to this variant, the polysaccharide is preferably hyaluronic acid, or a salt thereof such as the sodium salt (sodium hyaluronate).

II. Synthetic Polymeric Gelling Agents

For the purposes of the invention, the term “synthetic” means that the polymer is neither naturally existing nor a derivative of a polymer of natural origin.

The synthetic polymeric hydrophilic gelling agent under consideration according to the invention may or may not be particulate.

For the purposes of the invention, the term “particulate” means that the polymer is in the form of particles, preferably spherical particles.

As emerges from the text hereinbelow, the polymeric hydrophilic gelling agent is advantageously chosen from crosslinked acrylic homopolymers or copolymers; polyacrylamides and cros slinked and/or neutralized 2-acrylamido-2-methylpropanesulfonic acid polymers and copolymers; modified or unmodified carboxyvinyl polymers, and mixtures thereof, especially as defined below.

II.A. Particulate Synthetic Polymeric Gelling Agents

They are preferably chosen from crosslinked polymers.

They may especially be crosslinked acrylic homopolymers or copolymers, which are preferably partially neutralized or neutralized, and which are in particulate form.

According to one embodiment, the particulate gelling agent according to the present invention is chosen from crosslinked sodium polyacrylates. Preferably, it has in the dry or non-hydrated state a mean size of less than or equal to 100 μm and preferably less than or equal to 50 μm. The mean size of the particles corresponds to the mass-mean diameter measured by laser particle size analysis or another equivalent method known to those skilled in the art.

Thus, preferably, the particulate gelling agent according to the present invention is chosen from crosslinked sodium polyacrylates, preferably in the form of particles with a mean size (or mean diameter) of less than or equal to 100 microns, more preferably in the form of spherical particles.

As examples of crosslinked sodium polyacrylates, mention may be made of those sold under the brand names Octacare X100, X110 and RM100 by the company Avecia, those sold under the names Flocare GB300 and Flosorb 500 by the company SNF, those sold under the names Luquasorb 1003, Luquasorb 1010, Luquasorb 1280 and Luquasorb 1110 by the company BASF, those sold under the names Water Lock G400 and G430 (INCI name: Acrylamide/Sodium acrylate copolymer) by the company Grain Processing.

Mention may also be made of crosslinked polyacrylate microspheres, for instance those sold under the name Aquakeep® 10 SH NF by the company Sumitomo Seika.

Such gelling agents may be used in a proportion of from 0.1% to 5% by weight of solids relative to the total weight of the aqueous phase, especially from 0.5% to 2% by weight and in particular in a proportion of about from 0.8% to 1.7% by weight, relative to the total weight of the aqueous phase.

II.B. Non-Particulate Synthetic Polymeric Gelling Agents

This family of gelling agents may be detailed under the following subfamilies:

1. Polyacrylamides and crosslinked and/or neutralized 2-acrylamido-2-methylpropanesulfonic acid polymers and copolymers, and

2. Modified or unmodified carboxyvinyl polymers.

II.B.1 Polyacrylamides and Crosslinked and/or Neutralized 2-acrylamido-2-methylpropanesulfonic Acid Polymers and Copolymers

The polymers used that are suitable as aqueous gelling agent for the invention may be crosslinked or non-crosslinked homopolymers or copolymers comprising at least the 2-acrylamidomethylpropanesulfonic acid (AMPS®) monomer, in a form partially or totally neutralized with a mineral base such as sodium hydroxide or potassium hydroxide.

They are preferably totally or almost totally neutralized, i.e. at least 90% neutralized.

These AMPS® polymers according to the invention may be crosslinked or non-crosslinked.

When the polymers are crosslinked, the crosslinking agents may be chosen from the polyolefinically unsaturated compounds commonly used for crosslinking polymers obtained by radical polymerization.

Examples of crosslinking agents that may be mentioned include divinylbenzene, diallyl ether, dipropylene glycol diallyl ether, polyglycol diallyl ethers, triethylene glycol divinyl ether, hydroquinone diallyl ether, ethylene glycol or tetraethylene glycol di(meth)acrylate, trimethylolpropane triacrylate, methylenebisacrylamide, methylenebismethacrylamide, triallylamine, triallyl cyanurate, diallyl maleate, tetraallylethylenediamine, tetraallyloxyethane, trimethylolpropane diallyl ether, allyl (meth)acrylate, allylic ethers of alcohols of the sugar series, or other allylic or vinyl ethers of polyfunctional alcohols, and also the allylic esters of phosphoric and/or vinylphosphonic acid derivatives, or mixtures of these compounds.

According to one preferred embodiment of the invention, the crosslinking agent is chosen from methylenebisacrylamide, allyl methacrylate and trimethylolpropane triacrylate (TMPTA). The degree of crosslinking generally ranges from 0.01 mol % to 10 mol % and more particularly from 0.2 mol % to 2 mol % relative to the polymer.

The AMPS® polymers that are suitable for use in the invention are water-soluble or water-dispersible. In this case, they are:
either “homopolymers” comprising only AMPS monomers and, if they are crosslinked, one or more crosslinking agents such as those defined above;
or copolymers obtained from AMPS ® and from one or more hydrophilic or hydrophobic ethylenically unsaturated monomers and, if they are crosslinked, one or more crosslinking agents such as those defined above. When said copolymers comprise hydrophobic ethylenically unsaturated monomers, these monomers do not comprise a fatty chain and are preferably present in small amounts.

For the purpose of the present invention, the term “fatty chain” means any hydrocarbon-based chain comprising at least 7 carbon atoms.

The term “water-soluble or water-dispersible” means polymers which, when introduced into an aqueous phase at 25° C., at a mass concentration equal to 1%, make it possible to obtain a macroscopically homogeneous and transparent solution, i.e. a solution with a maximum light transmittance value, at a wavelength equal to 500 nm, through a sample 1 cm thick, of at least 60% and preferably of at least 70%.

The “homopolymers ” according to the invention are preferably crosslinked and neutralized, and they may be obtained according to the preparation process comprising the following steps:

(a) the monomer such as AMPS in free form is dispersed or dissolved in a solution of tert-butanol or of water and tert-butanol;

(b) the monomer solution or dispersion obtained in (a) is neutralized with one or more mineral or organic bases, preferably aqueous ammonia NH₃, in an amount making it possible to obtain a degree of neutralization of the sulfonic acid functions of the polymer ranging from 90% to 100%;

(c) the crosslinking monomer(s) are added to the solution or dispersion obtained in (b);

(d) a standard free-radical polymerization is performed in the presence of free-radical initiators at a temperature ranging from 10° C. to 150° C.; the polymer precipitates from the tert-butanol-based solution or dispersion.

The water-soluble or water-dispersible AMPS® copolymers according to the invention contain water-soluble ethylenically unsaturated monomers, hydrophobic monomers, or mixtures thereof.

The water-soluble comonomers may be ionic or nonionic.

Among the ionic water-soluble comonomers, examples that may be mentioned include the following compounds, and salts thereof:
(meth)acrylic acid,
styrenesulfonic acid,
vinylsulfonic acid and (meth)allylsulfonic acid,
vinylphosphonic acid,
maleic acid,
itaconic acid,
crotonic acid,
water-soluble vinyl monomers of formula (A) below:

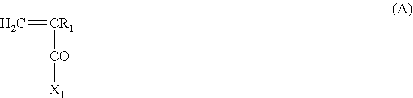

in which:
R₁ is chosen from H, —CH₃, —C₂H₅ and —C₃H₇,
X₁ is chosen from:
alkyl oxides of type —OR₂ where R₂ is a linear or branched, saturated or unsaturated hydrocarbon-based radical containing from 1 to 6 carbon atoms, substituted with at least one sulfonic (—SO₃—) and/or sulfate (—SO₄—) and/or phosphate (—PO₄H₂—) group.

Among the nonionic water-soluble comonomers, examples that may be mentioned include:
(meth)acrylamide,
N-vinylacetamide and N-methyl-N-vinylacetamide,
N-vinylformamide and N-methyl-N-vinylformamide,
maleic anhydride,
vinylamine,
N-vinyllactams comprising a cyclic alkyl group containing from 4 to 9 carbon atoms, such as N-vinylpyrrolidone, N-butyrolactam and N-vinylcaprolactam,
vinyl alcohol of formula CH₂═CHOH,
water-soluble vinyl monomers of formula (B) below:

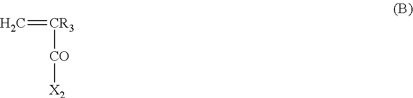

in which:
R₃ is chosen from H, —CH₃, —C₂H₅ and —C₃H₇,
X₂ is chosen from alkyl oxides of the type —OR₄ where R₄ is a linear or branched, saturated or unsaturated hydrocarbon-based radical containing from 1 to 6 carbon atoms, optionally substituted with a halogen (iodine, bromine, chlorine or fluorine) atom; a hydroxyl (—OH) group; ether.

Mention is made, for example, of glycidyl (meth)acrylate, hydroxyethyl methacrylate, and (meth)acrylates of ethylene glycol, of diethylene glycol or of polyalkylene glycol.

Among the hydrophobic comonomers without a fatty chain, mention may be made, for example, of:
styrene and derivatives thereof, such as 4-butylstyrene, α-methylstyrene and vinyltoluene;
vinyl acetate of formula CH₂═CH—OCOCH₃;
vinyl ethers of formula CH₂═CHOR in which R is a linear or branched, saturated or unsaturated hydrocarbon-based radical containing from 1 to 6 carbon atoms;
acrylonitrile;
caprolactone;
vinyl chloride and vinylidene chloride;
silicone derivatives, which, after polymerization, result in silicone polymers such as methacryloxypropyltris(trimethylsiloxy)silane and silicone methacrylamides;
hydrophobic vinyl monomers of formula (C) below:

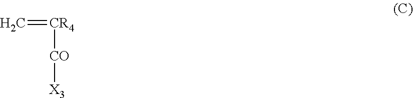

in which:
R₄ is chosen from H, —CH₃, —C₂H₅ and —C₃H₇;
X₃ is chosen from:
alkyl oxides of the type —OR₅ where R₅ is a linear or branched, saturated or unsaturated hydrocarbon-based radical containing from 1 to 6 carbon atoms.

Mention is made, for example, of methyl methacrylate, ethyl methacrylate, n-butyl (meth)acrylate, tert-butyl (meth)acrylate, cyclohexyl acrylate, isobornyl acrylate and 2-ethylhexyl acrylate.

The water-soluble or water-dispersible AMPS® polymers of the invention preferably have a molar mass ranging from 50 000 g/mol to 10 000 000 g/mol, preferably from 80 000 g/mol to 8 000 000 g/mol, and even more preferably from 100 000 g/mol to 7 000 000 g/mol.

As water-soluble or water-dispersible AMPS homopolymers suitable for use in the invention, mention may be made, for example, of crosslinked or non-crosslinked polymers of sodium acrylamido-2-methylpropanesulfonate, such as that used in the commercial product Simulgel 800 (CTFA name: Sodium Polyacryloyldimethyl Taurate), crosslinked ammonium acrylamido-2-methylpropanesulfonate polymers (INCI name: Ammonium Polydimethyltauramide) such as those described in patent EP 0 815 928 B1 and such as the product sold under the trade name Hostacerin AMPS® by the company Clariant.

As preferred water-soluble or water-dispersible AMPS homopolymers in accordance with the invention, mention may be made of crosslinked ammonium acrylamido-2-methylpropanesulfonate polymers.

As water-soluble or water-dispersible AMPS copolymers in accordance with the invention, examples that may be mentioned include:
crosslinked acrylamide/sodium acrylamido-2-methylpropanesulfonate copolymers, such as that used in the commercial product Sepigel 305 (CTFA name: Polyacrylamide/C₁₃-C₁₄ Isoparaffin/Laureth-7) or that used in the commercial product sold under the name Simulgel 600 (CTFA name: Acrylamide/Sodium acryloyldimethyltaurate/Isohexadecane/Polysorbate-80) by the company SEPPIC;
copolymers of AMPS® and of vinylpyrrolidone or vinylformamide, such as that used in the commercial product sold under the name Aristoflex AVC® by the company Clariant (CTFA name: Ammonium Acryloyldimethyltaurate/VP copolymer) but neutralized with sodium hydroxide or potassium hydroxide;
copolymers of AMPS® and of sodium acrylate, for instance the AMPS/sodium acrylate copolymer, such as that used in the commercial product sold under the name Simulgel EG® by the company SEPPIC;
copolymers of AMPS® and of hydroxyethyl acrylate, for instance the AMPS®/hydroxyethyl acrylate copolymer, such as that used in the commercial product sold under the name Simulgel NS® by the company SEPPIC (CTFA name: Hydroxyethyl acrylate/sodium acryloyldimethyltaurate copolymer (and) squalane (and) polysorbate 60), or such as the product sold under the name Sodium acrylamido-2-methylpropanesulfonate/hydroxyethyl acrylate copolymer, such as the commercial product Sepinov EM or Sepinov EMT 10 (INCI name: Hydroxyethyl acrylate/Sodium acryloyldimethyltaurate copolymer).

As preferred water-soluble or water-dispersible AMPS copolymers in accordance with the invention, mention may be made of copolymers of AMPS® and of hydroxyethyl acrylate.

In general, an aqueous phase according to the invention may comprise from 0.1% to 12% by weight, preferably from 0.3% to 10% by weight and more preferentially from 0.5% to 8% by weight of solids of polyacrylamide(s) and/or of crosslinked and/or neutralized 2-acrylamido-2-methylpropanesulfonic acid polymer(s) and copolymer(s) relative to its total weight.

II.B.2 Modified or Unmodified Carboxyvinyl Polymers

The modified or unmodified carboxyvinyl polymers may be copolymers derived from the polymerization of at least one monomer (a) chosen from α,β-ethylenically unsaturated carboxylic acids or esters thereof, with at least one ethylenically unsaturated monomer (b) comprising a hydrophobic group.

The term “copolymers” means both copolymers obtained from two types of monomer and those obtained from more than two types of monomer, such as terpolymers obtained from three types of monomer.

In particular, among the modified or unmodified carboxyvinyl polymers, mention may also be made of sodium polyacrylates such as those sold under the name Cosmedia SP® containing 90% solids and 10% water, or Cosmedia SPL® as an inverse emulsion containing about 60% solids, an oil (hydrogenated polydecene) and a surfactant (PPG-5 Laureth-5), both sold by the company Cognis.

Mention may also be made of partially neutralized sodium polyacrylates that are in the form of an inverse emulsion comprising at least one polar oil, for example the product sold under the name Luvigel® EM sold by the company BASF.

The modified or unmodified carboxyvinyl polymers may also be chosen from crosslinked (meth)acrylic acid homopolymers.

For the purposes of the present patent application, the term “(meth)acrylic” means “acrylic or methacrylic”.

Examples that may be mentioned include the products sold by Lubrizol under the names Carbopol 910, 934, 940, 941, 934 P, 980, 981, 2984, 5984 and Carbopol Ultrez 10 Polymer, or by 3V-Sigma under the name Synthalen® K, Synthalen® L or Synthalen® M.

Among the modified or unmodified carboxyvinyl polymers, mention may be made in particular of Carbopol (CTFA name: carbomer) and Pemulen (CTFA name: Acrylates/C₁₀₋₃₀ alkyl acrylate crosspolymer) sold by the company Lubrizol.

The modified or unmodified carboxyvinyl polymers may be present in a proportion of from 0.1% to 10% by weight of solids relative to the weight of the aqueous phase, in particular from 0.3% to 8% by weight and preferably between 0.4% and 6% by weight, relative to the weight of the aqueous phase.

Advantageously, a composition according to the invention comprises at least one synthetic polymeric gelling agent, preferably chosen from crosslinked and/or neutralized 2-acrylamido-2-methylpropanesulfonic acid polymers and copolymers and modified or unmodified carboxyvinyl polymers.

According to a preferred variant, the synthetic polymeric hydrophilic gelling agent is chosen from crosslinked ammonium acrylamido-2-methylpropanesulfonate polymers, copolymers of AMPS® and of hydroxyethyl acrylate, and crosslinked (meth)acrylic acid homopolymers, preferably copolymers of AMPS® and of hydroxyethyl acrylate.

III. Other Hydrophilic Gelling Agents

These gelling agents are more particularly chosen from mixed silicates and fumed silicas.

IIIA. Mixed Silicate

For the purposes of the present invention, the term “mixed silicate” means all silicates of natural or synthetic origin containing several (two or more) types of cations chosen from alkali metals (for example Na, Li, K) or alkaline-earth metals (for example Be, Mg, Ca), transition metals and aluminum.

According to a particular embodiment, the mixed silicate(s) are in the form of solid particles containing at least 10% by weight of at least one silicate relative to the total weight of the particles. In the rest of the present description, these particles are referred to as “silicate particles”.

Preferably, the silicate particles contain less than 1% by weight of aluminum relative to the total weight of the particles. Even more preferably, they contain from 0% to 1% by weight of aluminum relative to the total weight of the particles.

Preferably, the silicate particles contain at least 50% by weight of silicate and better still at least 70% by weight relative to the total weight of the particles. Particles containing at least 90% by weight of silicates, relative to the total weight of the particles, are particularly preferred.

In particular, it is an alkali metal or alkaline-earth metal, aluminum or iron silicate or mixture of silicates.

Preferably, it is sodium, magnesium and/or lithium silicate.

To ensure good cosmetic properties, these silicates are generally in a finely divided form, and in particular in the form of particles with a mean size ranging from 2 nm to 1 μm (from 2 nm to 1000 nm), preferably from 5 nm to 600 nm and even more preferentially from 20 to 250 nm.

The silicate particles may have any form, for example the form of spheres, flakes, needles, platelets, disks, leaflets, or totally random forms. Preferably, the silicate particles are in the form of disks or leaflets.

Thus, the term “mean size” of the particles means the numerical mean size of the largest dimension (length) that it is possible to measure between two diametrically opposite points on an individual particle. The size may be determined, for example, by transmission electron microscopy or by measuring the specific surface area via the BET method or by laser particle size analysis.

When the particles are in the form of disks or leaflets, they generally have a thickness ranging from about 0.5 nm to 5 nm.

The silicate particles may consist of an alloy with metal or metalloid oxides, obtained, for example, by thermal melting of the various constituents thereof. When the particles also comprise such a metal or metalloid oxide, this oxide is preferably chosen from silicon, boron or aluminum oxide.

According to a particular embodiment of the invention, the silicates are phyllosilicates, namely silicates having a structure in which the SiO₄ tetrahedra are organized in leaflets between which the metal cations are enclosed.

The mixed silicates that are suitable for use in the invention may be chosen, for example, from montmorillonites, hectorites, bentonites, beidellite and saponites. According to a preferred embodiment of the invention, the mixed silicates used are more particularly chosen from hectorites and bentonites, and better still from laponites.

A family of silicates that is particularly preferred in the compositions of the present invention is thus the laponite family. Laponites are sodium magnesium silicates also possibly containing lithium, which have a layer structure similar to that of montmorillonites. Laponite is the synthetic form of the natural mineral known as hectorite. The synthetic origin of this family of silicates is of considerable advantage over the natural form, since it allows good control the composition of the product. In addition, laponites have the advantage of having a particle size that is much smaller than that of the natural minerals hectorite and bentonite.

Laponites that may especially be mentioned include the products sold under the following names: Laponite® XLS, Laponite® XLG, Laponite® RD, Laponite® RDS, Laponite® XL21 (these products are sodium magnesium silicates and sodium lithium magnesium silicates) by the company Rockwood Additives Limited.

Such gelling agents may be used in a proportion of from 0.1% to 8% by weight of solids relative to the total weight of the aqueous phase, especially from 0.1% to 5% by weight and in particular from 0.5% to 3% by weight, relative to the total weight of the aqueous phase.

III.B. Hydrophilic Fumed Silica

The fumed silicas according to the present invention are hydrophilic.

The hydrophilic fumed silicas are obtained by pyrolysis of silicon tetrachloride (SiCl₄) in a continuous flame at 1000° C. in the presence of hydrogen and oxygen. Among the fumed silicas of hydrophilic nature that may be used according to the present invention, mention may especially be made of those sold by the company Degussa or Evonik Degussa under the trade names Aerosil® 90, 130, 150, 200, 300 and 380 or alternatively by the company Cabot under the name Carbosil H5.

Such gelling agents may be used in a proportion of from 0.1% to 10% by weight of solids relative to the total weight of the aqueous phase, especially from 0.1% to 5% by weight and in particular from 0.5% to 3% by weight, relative to the total weight of the aqueous phase.

Starchy Polysaccharides

According to a particular embodiment, the composition according to the present invention may also comprise at least one additional hydrophilic gelling agent chosen from starchy polysaccharides.

The starchy additional gelling agent is water-soluble or water-dispersible.

As representatives of this category, mention may be made most particularly of native starches and modified starches.

Native Starches

The starches that may be used in the present invention are more particularly macromolecules in the form of polymers consisting of elementary moieties which are anhydroglucose units (dextrose), linked via α(1,4) bonds of chemical formula (C₆H₁₀O₅)ₙ. The number of these moieties and their assembly make it possible to distinguish amylose, a molecule formed from about 600 to 1000 linearly linked glucose units, and amylopectin, a polymer branched approximately every 25 glucose residues (α(1,6) bond). The total chain may include between 10 000 and 100 000 glucose residues.

Starch is described in particular in Kirk-Othmer's Encyclopaedia of Chemical Technology, 3rd edition, volume 21, pages 492-507, Wiley Interscience, 1983.

The relative proportions of amylose and of amylopectin, and their degree of polymerization, vary as a function of the botanical origin of the starches. On average, a sample of native starch consists of about 25% amylose and 75% amylopectin.

Occasionally, phytoglycogen is present (between 0% and 20% of the starch), which is an analog of amylopectin but branched every 10 to 15 glucose residues.

Starch may be in the form of semicrystalline granules: amylopectin is organized in leaflets, amylose forms a less well organized amorphous zone between the various leaflets.

Amylose is organized in a straight helix with six glucoses per turn. It dissociates into assimilable glucose under the action of enzymes, amylases, all the more easily when it is in amylopectin form. Specifically, the helical formation does not promote the accessibility of starch to the enzymes.

Starches are generally in the form of a white powder, which is insoluble in cold water, whose elemental particle size ranges from 3 to 100 microns.

By treating it with hot water, starch paste is obtained. It is exploited in industry for its thickening and gelling properties.

The botanical origin of the starch molecules used in the present invention may be cereals or tubers. Thus, the starches are chosen, for example, from corn starch, rice starch, cassava starch, tapioca starch, barley starch, potato starch, wheat starch, sorghum starch and pea starch.

The native starches are represented, for example, by the products sold under the names C*Amilogel™, Cargill Gel™, C* Gel™, Cargill Gum™, DryGel™ and C*Pharm Gel™ by the company Cargill, under the name Corn Starch by the company Roquette, and under the name Tapioca Pure by the company National Starch.

Modified Starches

The modified starches used in the composition of the invention may be modified via one or more of the following reactions: pregelatinization, degradation (acid hydrolysis, oxidation, dextrinization), substitution (esterification, etherification), crosslinking (esterification), bleaching.

More particularly, these reactions may be performed in the following manner:
pregelatinization by splitting the starch granules (for example drying and cooking in a drying drum);
acid hydrolysis giving rise to very rapid retrogradation on cooling;
oxidation with strong oxidizing agents (alkaline medium, in the presence of sodium hypochlorite NaOCl for example) leading to the polymerization of the starch molecule and to the introduction of carboxyl groups into the starch molecule (mainly oxidation of the hydroxyl group at C₆);
dextrinization in acid medium at high temperature (hydrolysis followed by repolymerization);
crosslinking with functional agents capable of reacting with the hydroxyl groups of the starch molecules, which will thus bond together (for example with glyceryl and/or phosphate groups);
esterification in alkaline medium for the grafting of functional groups, especially C₁-C₆ acyl (acetyl), C₁-C₆ hydroxyalkyl (hydroxyethyl or hydroxypropyl), carboxymethyl or octenylsuccinic.

Monostarch phosphates (of the type St-O—PO—(OX)₂), distarch phosphates (of the type St-O—PO—(OX)—O-St) or even tristarch phosphates (of the type St-O—PO—(O-St)₂) or mixtures thereof may especially be obtained by crosslinking with phosphorus compounds.

X in particular denotes alkali metals (for example sodium or potassium), alkaline-earth metals (for example calcium or magnesium), ammonium salts, amine salts, for instance those of monoethanolamine, diethanolamine, triethanolamine, 3-amino-1,2-propanediol, or ammonium salts derived from basic amino acids such as lysine, arginine, sarcosine, ornithine or citrulline.

The phosphorus compounds may be, for example, sodium tripolyphosphate, sodium orthophosphate, phosphorus oxychloride or sodium trimetaphosphate.

According to the invention, it is also possible to use amphoteric starches, these amphoteric starches containing one or more anionic groups and one or more cationic groups. The anionic and cationic groups may be linked to the same reactive site of the starch molecule or to different reactive sites; they are preferably linked to the same reactive site. The anionic groups may be of carboxylic, phosphate or sulfate type, preferably carboxylic. The cationic groups may be of primary, secondary, tertiary or quaternary amine type.

The amphoteric starches are in particular chosen from the compounds having the following formulaw:

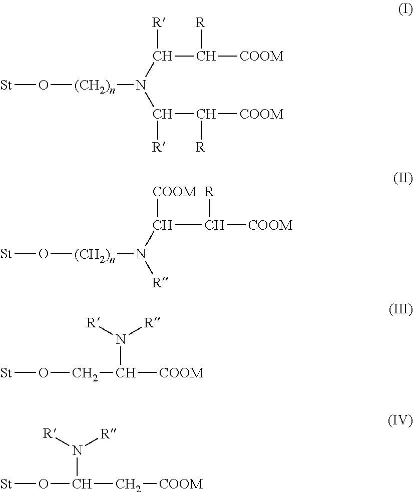

in which:
St-O represents a starch molecule;
R, which may be identical or different, represents a hydrogen atom or a methyl radical;
R′, which may be identical or different, represents a hydrogen atom, a methyl radical or a -COOH group;
n is an integer equal to 2 or 3;
M, which may be identical or different, denotes a hydrogen atom, an alkali metal or alkaline-earth metal such as Na, K, Li or NH₄, a quaternary ammonium or an organic amine,
R″ represents a hydrogen atom or an alkyl radical containing from 1 to 6 carbon atoms.

These compounds are especially described in patents U.S. Pat. No. 5,455,340 and U.S. Pat. No. 4,017,460.

The starch molecules may be derived from any plant source of starch, especially such as corn, potato, oat, rice, tapioca, sorghum, barley or wheat. It is also possible to use the hydrolysates of the starches mentioned above.

The modified starches are represented, for example, by the products sold under the names C*Tex-Instant (pregelatinized adipate), C*StabiTex-Instant (pregelatinized phosphate), C*PolarTex-Instant (pregelatinized hydroxypropyl), C*Set (acid hydrolysis, oxidation), C*size (oxidation), C*BatterCrisp (oxidation), C*DrySet (dextrinization), C*Tex™ (acetyl distarch adipate), C*PolarTex™ (hydroxypropyl distarch phosphate), C* StabiTex™ (distarch phosphate, acetyl distarch phosphate) by the company Cargill, by distarch phosphates or compounds rich in distarch phosphate such as the product sold under the references Prejel VA-70-T AGGL (gelatinized hydroxypropyl cassava distarch phosphate) or Prejel TK1 (gelatinized cassava distarch phosphate) or Prejel 200 (gelatinized acetyl cassava distarch phosphate) by the company Avebe or Structure Zea from National Starch (gelatinized corn distarch phosphate).

As examples of oxidized starches, use will be made especially of those sold under the name C*size from the company Cargill.

The native or modified starches described above may be advantageously used in a proportion of from 0.1% to 8% by weight of solids and preferably at about 1% by weight, relative to the total weight of the aqueous phase.

Particulate Starches

Particulate starches that may be mentioned in particular include:
starches grafted with an acrylic polymer (homopolymer or copolymer) and especially with sodium polyacrylate, for instance those sold under the names Sanfresh ST100MC by the company Sanyo Chemical Industries or Makimousse 25, Makimousse 12 by the company Daito Kasei (INCI name: Sodium polyacrylate starch),
hydrolyzed starches grafted with an acrylic polymer (homopolymer or copolymer), and especially acryloacrylamide/sodium acrylate copolymer, for instance those sold under the names Water Lock A-240, A-180, B-204, D-223, A-100, C-200 and D-223 by the company Grain Processing (INCI name: Starch/acrylamide/sodium acrylate copolymer);
polymers based on

starch, gum and cellulose derivative, such as the product containing starch, and sodium carboxymethylcellulose, for instance the product sold under the name Lysorb 220 by the company Lysac.

Mention may be made most particularly of C₁-C₄ carboxyalkyl starches, also referred to hereinbelow as carboxyalkyl starch. These compounds are obtained by grafting carboxyalkyl groups onto one or more alcohol functions of starch, especially by reaction of starch and of sodium monochloroacetate in alkaline medium.

The carboxyalkyl groups are generally attached via an ether function, more particularly to carbon 1. The degree of substitution with carboxyalkyl units of the C₁-C₄ carboxyalkyl starch preferably ranges from 0.1 to 1 and more particularly from 0.15 to 0.5. The degree of substitution is defined according to the present invention as being the mean number of hydroxyl groups substituted with an ester or ether group per monosaccharide unit of the polysaccharide.

The carboxyalkyl starches are advantageously used in the form of salts and especially of salts of alkali metals or alkaline-earth metals such as Na, K, Li, NH₄, or salts of a quaternary ammonium or of an organic amine such as monoethanolamine, diethanolamine or triethanolamine. The (C₁-C₄) carboxyalkyl starches are advantageously, in the context of the present invention, carboxymethyl starches. The carboxymethyl starches preferably comprise units having the following formula:

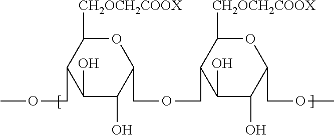

in which X, optionally covalently bonded to the carboxylic units, denotes a hydrogen atom, an alkali metal or alkaline-earth metal such as Na, K, Li, NH₄, a quaternary ammonium or an organic amine, for instance monoethanolamine, diethanolamine or triethanolamine.

Preferably, X denotes a cation Nat The carboxyalkyl starches that may be used according to the present invention are preferably non-pregelatinized carboxyalkyl starches. The carboxyalkyl starches that may be used according to the present invention are preferably partially or totally crosslinked carboxyalkyl starches.

In general, a crosslinked carboxyalkyl starch has, in contrast with a non-crosslinked carboxyalkyl starch, an increased, controllable viscosity of increased stability. The crosslinking thus makes it possible to reduce the syneresis phenomena and to increase the resistance of the gel to shear effects.

The carboxyalkyl starches under consideration according to the invention are more particularly potato carboxyalkyl starches. Thus, the carboxyalkyl starches that may be used according to the present invention are preferably sodium salts of carboxyalkyl starch, in particular a sodium salt of potato carboxymethyl starch, sold especially under the name Primojel® by the company DMV International or Glycolys® and Glycolys® LV by the company Roquette.

According to a particular mode, use will be made of the potato carboxymethyl starches sold especially under the name Glycolys® by the company Roquette. As stated previously, the C₁-C₄ carboxyalkyl starch particles are present in the compositions according to the invention in a swollen and non-split form. This swelling may be characterized by a swelling power Q which may advantageously be between 10 and 30 ml/g and preferably between 15 and 25 ml (volume of absorbed liquid)/g of dry particulate material.

Thus, the size of the swollen carboxyalkyl starch particles used according to the present invention generally ranges from 25 to 300 μm. For example, the gel Primojel® containing 10% by weight of potato carboxyalkyl starch and sodium salt in water contains more than 80% of swollen particles of this starch with a diameter of greater than 50 microns and more particularly greater than 100 microns.

According to a preferred embodiment variant of the invention, these particles are used for the preparation of the compositions according to the invention, in this swollen particulate state. To do so, these particles are advantageously used in the form of an aqueous gel either prepared beforehand or already commercially available. The gels under consideration according to the invention are advantageously translucent.

For example, a carboxymethyl starch gel such as Primojel® which is at a concentration of 10% by weight may be adjusted to the required concentration before being used for preparing the expected cosmetic composition.

Such a particulate starch may be used in a proportion of from 0.1% to 5% by weight of solids relative to the total weight of the aqueous phase, preferably between 0.5% and 2.5% by weight and in particular in a proportion of about 1.5% by weight, relative to the total weight of the aqueous phase.

Lipophilic Gelling Agent

For the purposes of the present invention, the term “lipophilic gelling agent” means a compound that is capable of gelling the oily phase of the compositions according to the invention.

For the purposes of the present invention, the term “non-cellulose-based lipophilic gelling agent” means a compound which is capable of gelling the oily phase of the compositions according to the invention and which does not comprise in its structure any cellulose groups of chemical formula:

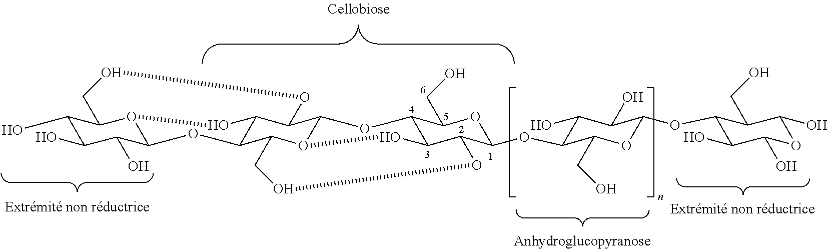

or any cellulose-based groups resulting from the reaction of the OH groups of cellulose with chemical reagents such as a cellulose ether (ethylcellulose), cellulose ester (carboxymethylcellulose) or cellulose ester-ether.

The lipophilic gelling agent is thus present in the oily phase of the composition.

The gelling agent is liposoluble or lipodispersible.

The non-cellulose-based lipophilic gelling agent may be chosen from particulate gelling agents other than apolar hydrocarbon-based waxes with a melting point of greater than 75.0° C., organopolysiloxane elastomers, semicrystalline polymers, dextrin esters, polymers containing hydrogen bonding other than silicone polyamides, and mixtures thereof.

More preferentially, the non-cellulose-based lipophilic gelling agent may be chosen from particulate gelling agents other than apolar hydrocarbon-based waxes with a melting point of greater than 75.0° C., semicrystalline polymers, dextrin esters, hydrocarbon-based polyamides, and mixtures thereof.

I. Particulate Gelling Agents

The particulate gelling agent used in the composition according to the invention is in the form of particles, preferably spherical particles.

As representative lipophilic particulate gelling agents that are suitable for use in the invention, mention may be made most particularly of polar and apolar waxes, modified clays, and silicas such as fumed silicas and hydrophobic silica aerogels.

Waxes

The waxes are defined above.

The waxes that may be used in the compositions according to the invention are chosen from waxes that are solid at room temperature of animal, plant, mineral or synthetic origin, and mixtures thereof.

The waxes, for the purposes of the invention, may be those used generally in the cosmetic or dermatological fields. They may especially be polar or apolar hydrocarbon-based waxes with a melting point of less than or equal to 75.0° C., or silicone and/or fluoro waxes, optionally comprising ester or hydroxide functions. They may also be of natural or synthetic origin.

a) Polar Wax

For the purposes of the present invention, the term “polar wax” means a wax whose solubility parameter at 25° C., δa, is other than 0 (J/cm³)^{1/2}.

In particular, the term “polar wax” means a wax whose chemical structure is formed essentially from, or even consists of, carbon and hydrogen atoms, and comprising at least one highly electronegative heteroatom such as an oxygen, nitrogen, silicon or phosphorus atom.

The polar waxes can in particular be hydrocarbon, fluorinated or silicone waxes.

Preferentially, the polar waxes may be hydrocarbon-based waxes.

The term “hydrocarbon-based wax” means a wax formed essentially from, or even constituted by, carbon and hydrogen atoms, and optionally oxygen and nitrogen atoms, and that does not contain any silicon or fluorine atoms. It may contain alcohol, ester, ether, carboxylic acid, amine and/or amide groups.

According to the invention, the term “ester wax” means a wax comprising at least one ester function. According to the invention, the term “alcohol wax” means a wax comprising at least one alcohol function, i.e. comprising at least one free hydroxyl (OH) group.

Use may in particular be made, as ester wax, of:
ester waxes, such as those chosen from:

i) waxes of formula R₁COOR₂ in which R₁ and R₂ represent linear, branched or cyclic aliphatic chains in which the number of atoms ranges from 10 to 50, which may contain a heteroatom such as O, N or P and whose melting point ranges from 25 to 120° C.;

ii) bis(1,1,1-trimethylolpropane) tetrastearate, sold under the name Hest 2T-4S® by the company Heterene;

iii) diester waxes of a dicarboxylic acid of general formula R³—(—OCO—R⁴—COO—R⁵), in which R³ and R⁵ are identical or different, preferably identical, and represent a C₄-C₃₀ alkyl group (alkyl group comprising from 4 to 30 carbon atoms) and R⁴ represents a linear or branched C₄-C₃₀ aliphatic group (alkyl group comprising from 4 to 30 carbon atoms) which may or may not comprise one or more unsaturations and which is preferably linear and unsaturated;

iv) mention may also be made of the waxes obtained by catalytic hydrogenation of animal or vegetable oils having linear or branched C₈-C₃₂ fatty chains, for example such as hydrogenated jojoba oil, hydrogenated sunflower oil, hydrogenated castor oil, hydrogenated coconut oil, and also the waxes obtained by hydrogenation of castor oil esterified with cetyl alcohol;

v) beeswax, synthetic beeswax, polyglycerolated beeswax, carnauba wax, candelilla wax, oxypropylenated lanolin wax, rice bran wax, ouricury wax, esparto grass wax, cork fiber wax, sugar cane wax, Japan wax, sumac wax, montan wax, orange wax, laurel wax, hydrogenated jojoba wax, sunflower wax, lemon wax, olive wax or berry wax.

According to another embodiment, the polar wax can be an alcohol wax. According to the invention, the term “alcohol wax” means a wax comprising at least one alcohol function, i.e. comprising at least one free hydroxyl (OH) group. Alcohol waxes that may be mentioned include for example the C₃₀-₅₀ alcohol wax Performacol® 550 Alcohol sold by the company New Phase Technologies, stearyl alcohol and cetyl alcohol.

b) Silicone Waxes

It is also possible to use silicone waxes, which may advantageously be substituted polysiloxanes, preferably of low melting point.

“Silicone wax” is understood to mean an oil comprising at least one silicon atom and in particular comprising Si—O groups.

Among the commercial silicone waxes of this type, mention may be made in particular of those sold under the names Abilwax 9800, 9801 or 9810 (Goldschmidt), KF910 and KF7002 (Shin-Etsu), or 176-1118-3 and 176-11481 (General Electric).

The silicone waxes that may be used may also be alkyl or alkoxy dimethicones, and also (C₂₀-C₆₀)alkyl dimethicones, in particular (C₃₀-C₄₅)alkyl dimethicones, such as the silicone wax sold under the name SF-1642 by the company GE-Bayer Silicones or _{C30-45} alkyl dimethylsilyl polypropylsilsesquioxane under the name SW-8005® C30 Resin Wax sold by the company Dow Corning.

The waxes, for the purposes of the invention, may be those used generally in the cosmetic or dermatological fields.

In the context of the present invention, particularly advantageous waxes that may be mentioned include carnauba wax, polyethylene waxes, jojoba wax, candelilla wax and silicone waxes, in particular candelilla wax.

They may be present in the oily phase in a proportion of from 0.5% to 30% by weight relative to the weight of the oily phase, for example between 5% and 20% of the oily phase and more particularly from 2% to 15% by weight relative to the weight of the oily phase.

Modified Clays

The composition according to the invention may comprise at least one lipophilic clay.

The clays may be natural or synthetic, and they are made lipophilic by treatment with an alkylammonium salt such as a C₁₀ to C₂₂ ammonium chloride, for example distearyldimethylammonium chloride.

They can be chosen from bentonites, in particular hectorites and montmorillonites, beidellites, saponites, nontronites, sepiolites, biotites, attapulgites, vermiculites and zeolites.

They are preferably chosen from hectorites.

Hectorites modified with a C₁₀ to C₂₂ ammonium salt and preferably a C₁₀ to C₂₂ ammonium chloride, such as hectorite modified with distearyldimethylammonium chloride, for instance the product sold under the name Bentone 38V® by the company Elementis or bentone gel in isododecane sold under the name Bentone Gel ISD V® (87% isododecane/10% disteardimonium hectorite/3% propylene carbonate) by the company Elementis, are preferably used as lipophilic clays.

Hectorites modified with a C₁₀ to C₂₂, ammonium salt are advantageously used, in particular hectorites modified with a C₁₀ to C₂₂, ammonium chloride.

The lipophilic clay may especially be present in a content ranging from 0.1% to 25% by weight, particular from 0.5% to 20% and more particularly from 1% to 18% by weight relative to the total weight of the oily phase.

Silicas

The oily phase of a composition according to the invention may also comprise, as gelling agent, a fumed silica or silica aerogel particles.

a) Fumed Silica

Fumed silica which has undergone a hydrophobic surface treatment is most particularly suitable for use in the invention. This is because it is possible to chemically modify the surface of the silica, by chemical reaction generating a reduction in the number of silanol groups present at the surface of the silica. It is especially possible to substitute silanol groups with hydrophobic groups: a hydrophobic silica is then obtained.

The hydrophobic groups may be:
trimethylsiloxyl groups, which are obtained in particular by treatment of fumed silica in the presence of hexamethyldisilazane. Silicas thus treated are known as “Silica silylate” according to the CTFA (8th edition, 2000). They are sold, for example, under the references Aerosil R812® by the company Degussa, and Cab-O-Sil TS-530® by the company Cabot;
dimethylsilyloxyl or polydimethylsiloxane groups, which are especially obtained by treating fumed silica in the presence of polydimethylsiloxane or dimethyldichlorosilane. Silicas thus treated are known as “silica dimethyl silylate” according to the CTFA (8th Edition, 2000). They are sold, for example, under the references Aerosil R972® and Aerosil R974® by the company Degussa, and Cab-O-Sil TS-610® and Cab-O-Sil TS-720® by the company Cabot.

The fumed silicas may be present in a composition according to the present invention in a content of between 0.1% and 40% by weight, more particularly between 1% and 15% by weight and even more particularly between 2% and 10% by weight relative to the total weight of the oily phase.

b) Hydrophobic Silica Aerogels

According to a particularly preferred variant, the oily phase of a composition according to the invention comprises, as gelling agent, at least silica aerogel particles.

Silica aerogels are porous materials obtained by replacing (by drying) the liquid component of a silica gel with air.

They are generally synthesized via a sol-gel process in a liquid medium and then dried, usually by extraction with a supercritical fluid, the one most commonly used being supercritical CO₂. This type of drying makes it possible to avoid shrinkage of the pores and of the material. The sol-gel process and the various drying operations are described in detail in Brinker C. J. and Scherer G. W., Sol-Gel Science, New York: Academic Press, 1990.

The hydrophobic silica aerogel particles used in the present invention have a specific surface area per unit mass (SM) ranging from 500 to 1500 m²/g, preferably from 600 to 1200 m²/g and better still from 600 to 800 m²/g, and a size expressed as the volume-mean diameter (D[0.5]) ranging from 1 to 1500 μm, better still from 1 to 1000 μm, preferably from 1 to 100 μm, in particular from 1 to 30 μm, more preferably from 5 to 25 μm, better still from 5 to 20 μm and even better still from 5 to 15 μm.

According to one embodiment, the hydrophobic silica aerogel particles used in the present invention have a size expressed as volume-mean diameter (D[0.5]) ranging from 1 to 30 μm, preferably from 5 to 25 μm, better still from 5 to 20 μm and even better still from 5 to 15 μm.

The specific surface area per unit mass may be determined by the nitrogen absorption method, known as the BET (Brunauer-Emmett-Teller) method, described in The Journal of the American Chemical Society, vol. 60, page 309, February 1938 and corresponding to international standard ISO 5794/1 (annex D). The BET specific surface corresponds to the total specific surface of the particles under consideration.

The sizes of the silica aerogel particles may be measured by static light scattering using a commercial particle size analyzer such as the MasterSizer 2000 machine from Malvern. The data are processed on the basis of the Mie scattering theory. This theory, which is exact for isotropic particles, makes it possible to determine, in the case of non-spherical particles, an “effective” particle diameter. This theory is especially described in the publication by Van de Hulst, H. C., Light Scattering by Small Particles, Chapters 9 and 10, Wiley, New York, 1957.

According to an advantageous embodiment, the hydrophobic silica aerogel particles used in the present invention have a specific surface area per unit of mass (SM) ranging from 600 to 800 m²/g.

The silica aerogel particles used in the present invention may advantageously have a tapped density p ranging from 0.02 g/cm³ to 0.10 g/cm³, preferably from 0.03 g/cm³ to 0.08 g/cm³ and in particular ranging from 0.05 g/cm³ to 0.08 g/cm³.

In the context of the present invention, this density, known as the tapped density, may be assessed according to the following protocol:

40 g of powder are poured into a measuring cylinder; the measuring cylinder is then placed on a Stay 2003 machine from Stampf Volumeter; the measuring cylinder is then subjected to a series of 2500 tapping actions (this operation is repeated until the difference in volume between two consecutive tests is less than 2%); the final volume Vf of tapped powder is then measured directly on the measuring cylinder. The tapped density is determined by the ratio m/Vf, in this instance 40/Vf (Vf being expressed in cm³ and m in g).

According to a preferred embodiment, the hydrophobic silica aerogel particles used in the present invention have a specific surface area per unit of volume SV ranging from 5 to 60 m²/cm³, preferably from 10 to 50 m²/cm³ and better still from 15 to 40 m²/cm³.

The specific surface area per unit volume is given by the relationship: S_{V}=S_{M}×ρ; where ρ is the tapped density, expressed in g/cm³, and S_{M} is the specific surface area per unit of mass, expressed in m²/g, as defined above.

Preferably, the hydrophobic silica aerogel particles according to the invention have an oil-absorbing capacity, measured at the wet point, ranging from 5 to 18 ml/g, preferably from 6 to 15 ml/g and better still from 8 to 12 ml/g.

The absorption capacity measured at the wet point, denoted Wp, corresponds to the amount of oil which it is necessary to add to 100 g of particles in order to obtain a homogeneous paste.

It is measured according to the wet point method or the method for determining the oil uptake of a powder described in standard NF T 30-022. It corresponds to the amount of oil adsorbed onto the available surface of the powder and/or absorbed by the powder by measuring the wet point, described below:

An amount m=2 g of powder is placed on a glass plate, and the oil (isononyl isononanoate) is then added dropwise. After addition of 4 to 5 drops of oil to the powder, mixing is performed using a spatula, and addition of oil is continued until a conglomerate of oil and powder has formed. From this point, the oil is added at the rate of one drop at a time and the mixture is subsequently triturated with the spatula. The addition of oil is stopped when a firm, smooth paste is obtained. This paste must be able to be spread over the glass plate without cracks or the formation of lumps. The volume Vs (expressed in ml) of oil used is then noted.

The oil uptake corresponds to the ratio Vs/m.

The aerogels used according to the present invention are aerogels of hydrophobic silica, preferably of silylated silica (INCI name: silica silylate).

The term “hydrophobic silica” means any silica whose surface is treated with silylating agents, for example with halogenated silanes such as alkylchlorosilanes, siloxanes, in particular dimethylsiloxanes such as hexamethyldisiloxane, or silazanes, so as to functionalize the OH groups with silyl groups Si-Rn, for example trimethylsilyl groups.

As regards the preparation of hydrophobic silica aerogel particles that have been surface-modified by silylation, reference may be made to document U.S. Pat. No. 7,470,725.

Use will preferably be made of hydrophobic silica aerogel particles surface-modified with trimethylsilyl groups, preferably of the INCI name Silica silylate.

As hydrophobic silica aerogels that may be used in the invention, an example that may be mentioned is the aerogel sold under the name VM-2260 or VM-2270 (INCI name: Silica silylate) by the company Dow Corning, the particles of which have a mean size of about 1000 microns and a specific surface area per unit of mass ranging from 600 to 800 m²/g.

Mention may also be made of the aerogels sold by the company Cabot under the references Aerogel TLD 201, Aerogel OGD 201 and Aerogel TLD 203, Enova® Aerogel MT 1100 and Enova Aerogel MT 1200.

Use will preferably be made of the aerogel sold under the name VM-2270 (INCI name: Silica silylate) by the company Dow Corning, the particles of which have an average size ranging from 5-15 microns and a specific surface area per unit of mass ranging from 600 to 800 m²/g.

Such an aerogel advantageously makes it possible to promote the resistance of the deposit to sebum and to sweat.

Preferably, the hydrophobic silica aerogel particles are present in the composition according to the invention in a solids content ranging from 0.1% to 30% by weight, preferably from 0.2% to 20% by weight and preferably from 0.2% to 10% by weight relative to the total weight of the oily phase.

II. Organopolysiloxane Elastomer

According to another particularly preferred variant, the oily phase of a composition according to the invention comprises, as gelling agent, at least one organopolysiloxane elastomer.

The organopolysiloxane elastomer that may be used as lipophilic gelling agent has the advantage of giving the composition according to the invention good application properties. It affords a very soft feel and a matt effect after application, which is advantageous especially for application to the skin. It may also allow efficient filling of the hollows present on keratin materials.

The term “organopolysiloxane elastomer” or “silicone elastomer” means a supple, deformable organopolysiloxane with viscoelastic properties and especially with the consistency of a sponge or a supple sphere. Its modulus of elasticity is such that this material withstands deformation and has limited stretchability and contractability. This material is capable of regaining its original shape after stretching.

It is more particularly a crosslinked organopolysiloxane elastomer.

Thus, the organopolysiloxane elastomer may be obtained by crosslinking addition reaction of diorganopolysiloxane containing at least one hydrogen bonded to silicon and of diorganopolysiloxane containing ethylenically unsaturated groups bonded to silicon, especially in the presence of a platinum catalyst; or by dehydrogenation crosslinking condensation reaction between a diorganopolysiloxane comprising hydroxyl end groups and a diorganopolysiloxane containing at least one hydrogen bonded to silicon, especially in the presence of an organotin; or by crosslinking condensation reaction of a diorganopolysiloxane comprising hydroxyl end groups and of a hydrolysable organopolysilane; or by thermal crosslinking of organopolysiloxane, especially in the presence of an organoperoxide catalyst; or by crosslinking of organopolysiloxane via high-energy radiation such as gamma rays, ultraviolet rays or an electron beam.

Preferably, the organopolysiloxane elastomer is obtained by crosslinking addition reaction (A) of diorganopolysiloxane containing at least two hydrogens each bonded to a silicon, and (B) of diorganopolysiloxane containing at least two ethylenically unsaturated groups bonded to silicon, especially in the presence (C) of a platinum catalyst, as described, for instance, in patent application EP-A-295 886.

In particular, the organopolysiloxane elastomer may be obtained by reaction of dimethylpolysiloxane comprising dimethylvinylsiloxy end groups and of methylhydrogenopolysiloxane comprising trimethylsiloxy end groups, in the presence of a platinum catalyst.

Compound (A) is the base reagent for the formation of organopolysiloxane elastomer, and the crosslinking is performed by addition reaction of compound (A) with compound (B) in the presence of the catalyst (C).

Compound (A) is in particular an organopolysiloxane containing at least two hydrogen atoms bonded to different silicon atoms in each molecule.

Compound (A) may have any molecular structure, especially a linear-chain or branched-chain structure or a cyclic structure.

Compound (A) may have a viscosity at 25° C. ranging from 1 to 50 000 centistokes, especially so as to be readily miscible with compound (B).

The organic groups bonded to the silicon atoms of compound (A) may be alkyl groups such as methyl, ethyl, propyl, butyl, octyl; substituted alkyl groups such as 2-phenylethyl, 2-phenylpropyl or 3,3,3-trifluoropropyl; aryl groups such as phenyl, tolyl, xylyl; substituted aryl groups such as phenylethyl; and substituted monovalent hydrocarbon-based groups such as an epoxy group, a carboxylate ester group or a mercapto group.

Compound (A) may thus be chosen from trimethylsiloxy-terminated methylhydrogenopolysiloxanes, trimethylsiloxy-terminated dimethylsiloxane/methylhydrogenosiloxane copolymers, and dimethylsiloxane/methylhydrogenosiloxane cyclic copolymers.

Compound (B) is advantageously a diorganopolysiloxane containing at least two lower alkenyl groups (for example C₂-C₄); the lower alkenyl group may be chosen from vinyl, allyl and propenyl groups. These lower alkenyl groups may be located in any position on the organopolysiloxane molecule, but are preferably located at the ends of the organopolysiloxane molecule. The organopolysiloxane (B) may have a branched-chain, linear-chain, cyclic or network structure but the linear-chain structure is preferred. Compound (B) may have a viscosity ranging from the liquid state to the gum state. Preferably, compound (B) has a viscosity of at least 100 centistokes at 25° C.

Besides the abovementioned alkenyl groups, the other organic groups bonded to the silicon atoms in compound (B) may be alkyl groups such as methyl, ethyl, propyl, butyl or octyl; substituted alkyl groups such as 2-phenylethyl, 2-phenylpropyl or 3,3,3-trifluoropropyl; aryl groups such as phenyl, tolyl or xylyl; substituted aryl groups such as phenylethyl; and substituted monovalent hydrocarbon-based groups such as an epoxy group, a carboxylate ester group or a mercapto group.

The organopolysiloxanes (B) can be chosen from methylvinylpolysiloxanes, methylvinylsiloxane-dimethylsiloxane copolymers, dimethylpolysiloxanes comprising dimethylvinylsiloxy end groups, dimethylsiloxane-methylphenylsiloxane copolymers comprising dimethylvinylsiloxy end groups, dimethylsiloxane-diphenylsiloxane-methylvinylsiloxane copolymers comprising dimethylvinylsiloxy end groups, dimethylsiloxane-methylvinylsiloxane copolymers comprising trimethylsiloxy end groups, dimethylsiloxane-methylphenylsiloxane-methylvinylsiloxane copolymers comprising trimethylsiloxy end groups, methyl(3,3,3-trifluoropropyl)polysiloxanes comprising dimethylvinylsiloxy end groups, and dimethylsiloxane-methyl(3,3,3-trifluoropropyl)siloxane copolymers comprising dimethylvinylsiloxy end groups.

In particular, the organopolysiloxane elastomer can be obtained by reaction of dimethylpolysiloxane comprising dimethylvinylsiloxy end groups and of methylhydropolysiloxane comprising trimethylsiloxy end groups, in the presence of a platinum catalyst.

Advantageously, the sum of the number of ethylenic groups per molecule of compound (B) and of the number of hydrogen atoms bonded to silicon atoms per molecule of compound (A) is at least 5.

It is advantageous for compound (A) to be added in an amount such that the molecular ratio of the total amount of hydrogen atoms bonded to silicon atoms in compound (A) to the total amount of all the ethylenically unsaturated groups in compound (B) is within the range from 1.5/1 to 20/1.

Compound (C) is the catalyst for the crosslinking reaction, and is especially chloroplatinic acid, chloroplatinic acid-olefin complexes, chloroplatinic acid-alkenylsiloxane complexes, chloroplatinic acid-diketone complexes, platinum black and platinum on a support.

Catalyst (C) is preferably added in an amount of from 0.1 to 1000 parts by weight and better still from 1 to 100 parts by weight, as clean platinum metal, per 1000 parts by weight of the total amount of compounds (A) and (B).

The elastomer is advantageously a non-emulsifying elastomer.

The term “non-emulsifying” defines organopolysiloxane elastomers not containing any hydrophilic chains, and in particular not containing any polyoxyalkylene units (especially polyoxyethylene or polyoxypropylene) or any polyglyceryl units. Thus, according to a specific form of the invention, the composition comprises an organopolysiloxane elastomer devoid of polyoxyalkylene units and of polyglyceryl unit.

In particular, the silicone elastomer used in the present invention is chosen from Dimethicone Crosspolymer (INCI name), Vinyl Dimethicone Crosspolymer (INCI name), Dimethicone/Vinyl Dimethicone Crosspolymer (INCI name), Dimethicone Crosspolymer-3 (INCI name).

The organopolysiloxane elastomer particles may be conveyed in the form of a gel formed from an elastomeric organopolysiloxane included in at least one hydrocarbon-based oil and/or one silicone oil. In these gels, the organopolysiloxane particles are often nonspherical particles.

Non-emulsifying elastomers are described especially in patents EP 242 219, EP 285 886 and EP 765 656 and in patent application JP-A-61-194009.

The silicone elastomer is generally in the form of a gel, a paste or a powder, but advantageously in the form of a gel in which the silicone elastomer is dispersed in a linear silicone oil (dimethicone) or cyclic silicone oil (e.g.: cyclopentasiloxane), advantageously in a linear silicone oil.

Non-emulsifying elastomers that may be used more particularly include those sold under the names KSG-6, KSG-15, KSG-16, KSG-18, KSG-41, KSG-42, KSG-43 and KSG-44 by the company Shin-Etsu, DC9040 and DC9041 by the company Dow Corning, and SFE 839 by the company General Electric.

According to a particular mode, use is made of a gel of silicone elastomer dispersed in a silicone oil chosen from a non-exhaustive list comprising cyclopentadimethylsiloxane, dimethicones, dimethylsiloxanes, methyl trimethicone, phenyl methicone, phenyl dimethicone, phenyl trimethicone and cyclomethicone, preferably a linear silicone oil chosen from polydimethylsiloxanes (PDMS) or dimethicones with a viscosity at 25° C. ranging from 1 to 500 cSt, optionally modified with optionally fluorinated aliphatic groups, or with functional groups such as hydroxyl, thiol and/or amine groups.

Mention may be made especially of the compounds having the following INCI names:
dimethicone/vinyl dimethicone crosspolymer, such as USG-105 and USG-107A from the company Shin-Etsu; DC9506 and DC9701 from the company Dow Corning;
dimethicone/vinyl dimethicone crosspolymer (and) dimethicone, such as KSG-6 and KSG-16 from the company Shin-Etsu;
dimethicone/vinyl dimethicone crosspolymer (and) cyclopentasiloxane, such as KSG-15;
cyclopentasiloxane (and) dimethicone crosspolymer, such as DC9040, DC9045 and DC5930 from the company Dow Corning;
dimethicone (and) dimethicone crosspolymer, such as DC9041 from the company Dow Corning.
dimethicone (and) dimethicone crosspolymer, such as Dow Corning EL-9240® Silicone Elastomer Blend from the company Dow Corning (mixture of polydimethylsiloxane crosslinked with hexadiene/polydimethylsiloxane (2 cSt));
C₄₋₂₄ alkyl dimethicone/divinyl dimethicone crosspolymer, such as NuLastic Silk MA from the company Alzo.

As examples of silicone elastomers dispersed in a linear silicone oil that may advantageously be used according to the invention, mention may especially be made of the following references:
dimethicone/vinyl dimethicone crosspolymer (and) dimethicone, such as KSG-6 and KSG-16 from the company Shin-Etsu;
dimethicone (and) dimethicone crosspolymer, such as DC9041 from the company Dow Corning; and
dimethicone (and) dimethicone crosspolymer, such as Dow Corning EL-9240® Silicone Elastomer Blend from the company Dow Corning (mixture of polydimethylsiloxane crosslinked with hexadiene/polydimethylsiloxane (2 cSt)).
Diphenylsiloxy Phenyl Trimethicone (and) Dimethicone (and) Phenyl Vinyl Dimethicone Crosspolymer (INCI name), such as KSG 18A sold by the company Shin-Etsu.).

The organopolysiloxane elastomer particles may also be used in powder form: mention may be made especially of the powders sold under the names Dow Corning 9505 Powder and Dow Corning 9506 Powder by the company Dow Corning, these powders having the INCI name: dimethicone/vinyl dimethicone crosspolymer.

The organopolysiloxane powder may also be coated with silsesquioxane resin, as described, for example, in patent U.S. Pat. No. 5,538,793. Such elastomeric powders are sold under the names KSP-100, KSP-101, KSP-102, KSP-103, KSP-104 and KSP-105 by the company Shin-Etsu, and have the INCI name: vinyl dimethicone/methicone silsesquioxane crosspolymer.

As examples of organopolysiloxane powders coated with silsesquioxane resin that may advantageously be used according to the invention, mention may especially be made of the organopolysiloxane elastomers having the INCI name Vinyl Dimethicone/Methicone Silsesquioxane Crosspolymer, such as those sold under the commercial reference KSP-100 from the company Shin-Etsu.

As preferred lipophilic gelling agent of organopolysiloxane elastomer type, mention may be made especially of crosslinked organopolysiloxane elastomers chosen from Dimethicone Crosspolymer (INCI name), Vinyl Dimethicone Crosspolymer (INCI name), Dimethicone/Vinyl Dimethicone Crosspolymer (INCI name), Dimethicone Crosspolymer-3 (INCI name), Vinyl Dimethicone/Methicone Silsesquioxane Crosspolymer, Phenyl Vinyl Dimethicone Crosspolyer (INCI name) and in particular the Dimethicone Crosspolymer (INCI name).

As preferred lipophilic gelling agent of organopolysiloxane elastomer type, mention may be made especially of the products chosen from Dimethicone Crosspolymer (INCI name), Dimethicone (and) Dimethicone Crosspolymer (INCI name), Vinyl Dimethicone Crosspolymer (INCI name), Dimethicone/Vinyl Dimethicone Crosspolymer (INCI name), Dimethicone Crosspolymer-3 (INCI name), Vinyl Dimethicone/Methicone Silsesquioxane Crosspolymer, Diphenylsiloxy Phenyl Trimethicone (and) Dimethicone (and) Phenyl Vinyl Dimethicone Crosspolymer (INCI name) and in particular the Dimethicone Crosspolymer (INCI name).

The organopolysiloxane elastomer may be present in a composition of the present invention in a content of between 0.1% and 70% by weight of solids, especially between 0.2% and 60% by weight, advantageously between 0.5% and 40% and preferably from 1% to 20% by weight relative to the total weight of the oily phase.

III. Semicrystalline Polymers

The composition according to the invention may comprise at least one semicrystalline polymer. Preferably, the semicrystalline polymer has an organic structure, and a melting point of greater than or equal to 30° C.

For the purposes of the invention, the term “semicrystalline polymer” means polymers comprising a crystallizable portion and an amorphous portion and having a first-order reversible change of phase temperature, in particular of melting point (solid-liquid transition). The crystallizable part is either a side chain (or pendent chain) or a block in the backbone.

When the crystallizable portion of the semicrystalline polymer is a block of the polymer backbone, this crystallizable block has a chemical nature different than that of the amorphous blocks; in this case, the semicrystalline polymer is a block copolymer, for example of the diblock, triblock or multiblock type. When the crystallizable part is a chain that is pendent on the backbone, the semicrystalline polymer may be a homopolymer or a copolymer.

The melting point of the semicrystalline polymer is preferably less than 150° C.

The melting point of the semicrystalline polymer is preferably greater than or equal to 30° C. and less than 100° C. More preferably, the melting point of the semicrystalline polymer is greater than or equal to 30° C. and less than 70° C.

The semicrystalline polymer(s) according to the invention are solid at room temperature (25° C.) and atmospheric pressure (760 mmHg), with a melting point of greater than or equal to 30° C. The melting point values correspond to the melting point measured using a differential scanning calorimeter (DSC), such as the calorimeter sold under the name DSC 30 by the company Mettler, with a temperature rise of 5 or 10° C. per minute (the melting point under consideration is the point corresponding to the temperature of the most endothermic peak in the thermogram).

The semicrystalline polymer(s) according to the invention preferably have a melting point that is higher than the temperature of the keratin support intended to receive said composition, in particular the skin or the lips.

According to the invention, the semicrystalline polymers are advantageously soluble in the fatty phase, especially to at least 1% by weight, at a temperature that is higher than their melting point. Besides the crystallizable chains or blocks, the blocks of the polymers are amorphous.

For the purposes of the invention, the term “crystallizable chain or block” means a chain or block which, if it were alone, would change from the amorphous state to the crystalline state reversibly, depending on whether the temperature is above or below the melting point. For the purposes of the invention, a chain is a group of atoms, which are pendent or lateral relative to the polymer backbone. A block is a group of atoms belonging to the backbone, this group constituting one of the repeating units of the polymer.

Preferably, the polymer backbone of the semicrystalline polymers is soluble in the fatty phase at a temperature above their melting point.

Preferably, the crystallizable blocks or chains of the semicrystalline polymers represent at least 30% of the total weight of each polymer and better still at least 40%. The semicrystalline polymers bearing crystallizable side chains are homopolymers or copolymers. The semicrystalline polymers of the invention bearing crystallizable blocks are block or multiblock copolymers. They may be obtained by polymerizing a monomer bearing reactive (or ethylenic) double bonds or by polycondensation. When the polymers of the invention are polymers bearing crystallizable side chains, these side chains are advantageously in random or statistical form.

Preferably, the semicrystalline polymers of the invention are of synthetic origin.

According to one preferred embodiment, the semicrystalline polymer is chosen from:
homopolymers and copolymers comprising units resulting from the polymerization of one or more monomers bearing crystallizable hydrophobic side chain(s),
polymers bearing in the backbone at least one crystallizable block,
polycondensates of aliphatic or aromatic or aliphatic/aromatic polyester type,
copolymers of ethylene and propylene prepared via metallocene catalysis, and
acrylate/silicone copolymers.

The semicrystalline polymers that may be used in the invention may be chosen in particular from:
block copolymers of polyolefins of controlled crystallization, whose monomers are described in EP 0 951 897,
polycondensates, in particular of aliphatic or aromatic or aliphatic/aromatic polyester type,
copolymers of ethylene and propylene prepared via metallocene catalysis,
homopolymers or copolymers bearing at least one crystallizable side chain and homopolymers or copolymers bearing in the backbone at least one crystallizable block, such as those described in document U.S. Pat. No. 5,156,911, such as the (C₁₀-C₃₀)alkyl polyacrylates corresponding to the Intelimer® products from the company Landec described in the brochure Intelimer® Polymers, Landec IP22 (Rev. 4-97), for example the product Intelimer® IPA 13-1 from the company Landec, which is a polystearyl acrylate with a molecular weight of about 145 000 and a melting point of 49° C.,
homopolymers or copolymers bearing at least one crystallizable side chain, in particular containing fluoro group(s), as described in document WO 01/19333,
acrylate/silicone copolymers, such as copolymers of acrylic acid and of stearyl acrylate bearing polydimethylsiloxane grafts, copolymers of stearyl methacrylate bearing polydimethylsiloxane grafts, copolymers of acrylic acid and of stearyl methacrylate bearing polydimethylsiloxane grafts, copolymers of methyl methacrylate, butyl methacrylate, 2-ethylhexyl acrylate and stearyl methacrylate bearing polydimethylsiloxane grafts. Mention may be made in particular of the copolymers sold by the company Shin-Etsu under the names KP-561 (CTFA name: acrylates/dimethicone), KP-541 (CTFA name: acrylates/dimethicone and isopropyl alcohol), KP-545 (CTFA name: acrylates/dimethicone and cyclopentasiloxane),
and mixtures thereof.

Preferably, the amount of semicrystalline polymer(s), preferably chosen from semicrystalline polymers bearing crystallizable side chains, represents from 0.1% to 30% by weight of solids relative to the total weight of the oily phase, for example from 0.2% to 25% by weight, better still from 0.5% to 20% or even from 0.5% to 12% by weight, relative to the total weight of the oily phase.

IV. Dextrin Esters

The composition according to the invention may comprise as lipophilic gelling agent at least one dextrin ester.

In particular, the composition preferably comprises at least one preferably C₁₂ to C₂₄ and in particular C₁₄ to C₁₈ fatty acid ester of dextrin, or mixtures thereof.

Preferably, the dextrin ester is an ester of dextrin and of a C₁₂-C₁₈ and in particular C₁₄-C₁₈ fatty acid.

Preferably, the dextrin ester is chosen from dextrin myristate and/or dextrin palmitate, and mixtures thereof.

According to a particular embodiment, the dextrin ester is dextrin myristate, such as the product sold especially under the name Rheopearl MKL-2 by the company Chiba Flour Milling.

According to a preferred embodiment, the dextrin ester is dextrin palmitate. This product may be chosen, for example, from those sold under the names Rheopearl TL®, Rheopearl KL® and Rheopearl® KL2 by the company Chiba Flour Milling.

In a particularly preferred manner, the oily phase of a composition according to the invention may comprise from 0.1% to 30% by weight, preferably from 0.2% to 25% and preferably from 0.5% to 18% by weight of dextrin ester(s) relative to the total weight of the oily phase.

V. Polymers containing Hydrogen Bonding

As representatives of polymers containing hydrogen bonding that are suitable for use in the invention, mention may be made most particularly of hydrocarbon-based polyamides.

The oily phase of a composition according to the invention may comprise at least one hydrocarbon-based polyamide.

Preferably, the total content of hydrocarbon-based polyamide(s) is between 0.1% and 30% by weight expressed as solids, preferably between 1% and 20% by weight and preferably between 1% and 12% by weight relative to the total weight of the oily phase.

For the purposes of the invention, the term “polyamide” means a compound containing at least 2 amide repeating units, preferably at least 3 amide repeating units and better still 10 amide repeating units.

The term “hydrocarbon-based polyamide” means a polyamide formed essentially of, indeed even consisting of, carbon and hydrogen atoms, and optionally of oxygen or nitrogen atoms, and not comprising any silicon or fluorine atoms. It may contain alcohol, ester, ether, carboxylic acid, amine and/or amide groups.

For the purposes of the invention, the term “functionalized chain” means an alkyl chain comprising one or more functional groups or reagents chosen especially from hydroxyl, ether, ester, oxyalkylene and polyoxyalkylene groups.

Advantageously, this polyamide of the composition according to the invention has a weight-average molecular mass of less than 100 000 g/mol (especially ranging from 1000 to 100 000 g/mol), in particular less than 50 000 g/mol (especially ranging from 1000 to 50 000 g/mol) and more particularly ranging from 1000 to 30 000 g/mol, preferably from 2000 to 20 000 g/mol and better still from 2000 to 10 000 g/mol.

This polyamide is insoluble in water, especially at 25° C.

According to a first embodiment of the invention, the polyamide used is a polyamide of formula (I):

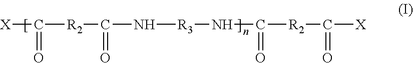

in which X represents a group —N(R₁)₂ or a group —OR₁ in which R₁ is a linear or branched C₈ to C₂₂, alkyl radical which may be identical or different, R₂ is a C₂₈-C₄₂ diacid dimer residue, R₃ is an ethylenediamine radical and n is between 2 and 5;

and mixtures thereof.

According to a particular mode, the polyamide used is an amide-terminated polyamide of formula (Ia):

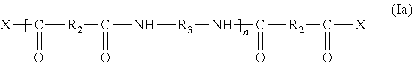

in which X represents a group —N(R₁)₂ in which R₁ is a linear or branched C₈ to C₂₂, alkyl radical which may be identical or different, R₂ is a C₂₈-C₄₂ diacid dimer residue, R₃ is an ethylenediamine radical and n is between 2 and 5;

and mixtures thereof.

The oily phase of a composition according to the invention may also comprise, additionally in this case, at least one additional polyamide of formula (Ib):

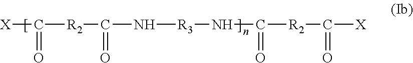

in which X represents a group —OR₁ in which R₁ is a linear or branched C₈ to C₂₂ and preferably C₁₆ to C₂₂, alkyl radical which may be identical or different, R₂ is a C₂₈-C₄₂ diacid dimer residue, R₃ is an ethylenediamine radical and n is between 2 and 5, such as the commercial products sold by the company Arizona Chemical under the names Uniclear 80 and Uniclear 100 or Uniclear 80 V, Uniclear 100 V and Uniclear 100 VG, the INCI name of which is Ethylenediamine/stearyl dimer dilinoleate copolymer.

Advantageously, the polymer bearing hydrogen bonding is the ethylene diamine/stearyl dimer dilinoleate copolymer.

VI. Hydrocarbon-Based Block Copolymer

Representative lipophilic gelling agents that may also be mentioned include other polymeric gelling agents, namely hydrocarbon-based block copolymers, also known as block copolymers.

The polymeric gelling agent is capable of thickening or gelling the hydrocarbon-based phase of the composition.

The term “amorphous polymer” means a polymer that does not have a crystalline form.

The polymeric gelling agent is also preferably film-forming, i.e. it is capable of forming a film when applied to the skin and/or the lips.

The hydrocarbon-based block copolymer may especially be a diblock, triblock, multiblock, radial or star copolymer, or mixtures thereof.

Such hydrocarbon-based block copolymers are described in patent application US-A-2002/005 562 and in patent U.S. Pat. No. 5,221,534.

The copolymer may contain at least one block whose glass transition temperature is preferably less than 20° C., preferably less than or equal to 0° C., preferably less than or equal to −20° C. and more preferably less than or equal to −40° C. The glass transition temperature of said block may be between −150° C. and 20° C. and especially between −100° C. and 0° C.

The hydrocarbon-based block copolymer present in the composition according to the invention is an amorphous copolymer formed by polymerization of an olefin. The olefin may especially be an elastomeric ethylenically unsaturated monomer.

Examples of olefins that may be mentioned include ethylenic carbide monomers, especially containing one or two ethylenic unsaturations and containing from 2 to 5 carbon atoms, such as ethylene, propylene, butadiene, isoprene or pentadiene.

Advantageously, the hydrocarbon-based block copolymer is an amorphous block copolymer of styrene and of an olefin.

Block copolymers comprising at least one styrene block and at least one block comprising units chosen from butadiene, ethylene, propylene, butylene and isoprene or a mixture thereof are especially preferred.

According to one preferred embodiment, the hydrocarbon-based block copolymer is hydrogenated to reduce the residual ethylenic unsaturations after the polymerization of the monomers.

In particular, the hydrocarbon-based block copolymer is a copolymer, optionally hydrogenated, containing styrene blocks and ethylene/C3-C4 alkylene blocks.

According to one preferred embodiment, the composition according to the invention comprises at least one diblock copolymer, which is preferably hydrogenated, preferably chosen from styrene-ethylene/propylene copolymers, styrene-ethylene/butadiene copolymers and styrene-ethylene/butylene copolymers. Diblock polymers are especially sold under the name Kraton® G1701E by the company Kraton Polymers.

Advantageously, a diblock copolymer such as those described previously is used as polymeric gelling agent, in particular a styrene-ethylene/propylene diblock copolymer or a mixture of diblock copolymers, as described previously.

Thus, according to a preferred implementation variant, a composition according to the invention comprises as lipophilic gelling agent at least one hydrocarbon-based block copolymer, preferably an optionally hydrogenated copolymer bearing styrene blocks and ethylene/C₃-C₄ alkylene blocks, even more preferentially a diblock copolymer, which is preferably hydrogenated, such as a styrene-ethylene/propylene copolymer or a styrene-ethylene/butadiene copolymer.

The hydrocarbon-based block copolymer (or the mixture of hydrocarbon-based block copolymers) may be present in a content ranging from 0.1% to 15% by weight, preferably ranging from 0.1% to 10% by weight, more preferentially ranging from 0.5% to 5% by weight and better still ranging from 0.5% to 3% by weight relative to the total weight of the composition.

More preferably, the non-cellulose-based lipophilic gelling agent is chosen from particulate gelling agents other than apolar hydrocarbon-based waxes with a melting point of greater than 75.0° C., in particular hydrophobic silica aerogels; organopolysiloxane elastomers, semicrystalline polymers, dextrin esters, polymers containing hydrogen bonding other than silicone polyamides; hydrocarbon-based block copolymers other than styrene-ethylene/propylene copolymers; and mixtures thereof.

According to an advantageous variant, a composition according to the invention comprises a lipophilic gelling agent chosen from organopolysiloxane elastomers, semicrystalline polymers, dextrin esters, hydrocarbon-based polyamides, block hydrocarbon-based copolymers which are preferably different from styrene-ethylene/propylene copolymers, particulate gelling agents chosen from polar waxes, hydrocarbon-based apolar waxes with a melting point of less than or equal to 75.0° C., silicone waxes, modified clays, silicas, especially hydrophobic silica aerogels, and also mixtures thereof.

According to a first preferred mode, the non-cellulose-based lipophilic gelling agent is chosen from hydrophobic silica aerogels.

According to a second preferred mode, the non-cellulose-based lipophilic gelling agent is chosen from organopolysiloxane elastomers.

According to a third preferred mode, the non-cellulose-based lipophilic gelling agent is chosen from modified clays.

Advantageously, a composition according to the invention comprises, as lipophilic gelling agent, a system comprising at least one modified clay and at least one hydrophobic silica aerogel.

Advantageously, a composition according to the invention comprises, as lipophilic gelling agent, a system comprising at least one modified clay and at least one organopolysiloxane elastomer.

Advantageously, a composition according to the invention comprises, as lipophilic gelling agent, a system comprising at least one hydrophobic silica aerogel and at least one organopolysiloxane elastomer.

Advantageously, a composition according to the invention comprises, as lipophilic gelling agent, a system comprising at least one modified clay at least one hydrophobic silica aerogel and at least one organopolysiloxane elastomer.

According to a preferred variant, a composition according to the invention comprises a lipophilic gelling agent chosen from:
semicrystalline homopolymers or copolymers bearing at least one crystallizable side chain and homopolymers or copolymers bearing in the backbone at least one crystallizable block, such as the (C₁₀-C₃₀)alkyl polyacrylates corresponding to the Intelimer® products from the company Landec, for example the product Intelimer® IPA 13-1 from the company Landec, which is a polystearyl acrylate with a molecular weight of about 145 000 and a melting point of 49° C.;
hydrocarbon-based polyamides and especially mixtures of amide-terminated polyamides of formula (Ia):

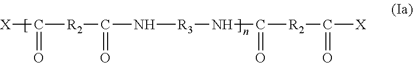

in which X represents a group —N(R₁)₂ in which R₁ is a linear or branched C₈ to C₂₂, alkyl radical which may be identical or different, R₂ is a C₂₈-C₄₂ diacid dimer residue, R₃ is an ethylenediamine radical and n is between 2 and 5 and of additional polyamides of formula (Ib):

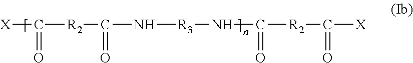

in which X represents a group —OR₁ in which R₁ is a linear or branched C₈ to C₂₂ and preferably C₁₆ to C₂₂, alkyl radical which may be identical or different, R₂ is a C₂₈-C₄₂ diacid dimer residue, R₃ is an ethylenediamine radical and n is between 2 and 5, such as the commercial products sold by the company Arizona Chemical under the names Uniclear 80 and Uniclear 100 or Uniclear 80 V, Uniclear 100 V and Uniclear 100 VG, the INCI name of which is Ethylenediamine/stearyl dimer dilinoleate copolymer;
hydrophobic silica aerogels;
hectorites modified with a salt, preferably a C₁₀ to C₂₂ ammonium chloride, such as hectorite modified with distearyldimethylammonium chloride, for instance the product sold under the name Bentone 38V® by the company Elementis or bentone gel in isododecane sold under the name Bentone Gel ISD V® (87% isododecane/10% disteardimonium hectorite/3% propylene carbonate) by the company Elementis;

organopolysiloxane elastomers.

According to a more preferred variant, a composition according to the invention comprises a lipophilic gelling agent chosen from:
semicrystalline homopolymers or copolymers bearing at least one crystallizable side chain and semicrystalline homopolymers or copolymers bearing at least one crystallizable block in the backbone,
hydrocarbon-based polyamides,
hydrophobic silica aerogels,
hectorites modified with a C₁₀ to C₂₂ ammonium chloride.

According to a particularly preferred variant, a composition according to the invention comprises a lipophilic gelling agent chosen from hectorites modified with a C₁₀ to C₂₂ ammonium chloride

organopolysiloxane elastomers.

According to a first particular variant, a composition according to the invention comprises:
at least one non-starchy hydrophilic gelling agent chosen from crosslinked and/or neutralized 2-acrylamido-2-methylpropanesulfonic acid (AMPS®) copolymers, and more particularly copolymers of AMPS® and of hydroxyethyl acrylate, and
at least one non-cellulose-based lipophilic gelling agent chosen from hectorites modified with a C10 to C22 ammonium chloride, and more particularly hectorites modified with distearyldimethylammonium chloride.

According to a second particular variant, the composition according to the invention comprises:
at least one non-starchy hydrophilic gelling agent chosen from crosslinked and/or neutralized 2-acrylamido-2-methylpropanesulfonic acid (AMPS®) copolymers, and more particularly copolymers of AMPS® and of hydroxyethyl acrylate, and
at least one lipophilic gelling agent chosen from hydrophobic silica aerogels,
at least one UV-screening agent,

and optionally an additional gelling agent chosen from particulate gelling agents, organopolysiloxane elastomers, semicrystalline polymers, dextrin esters, polymers bearing hydrogen bonding, hydrocarbon-based block copolymers, and mixtures thereof, and in particular a modified clay and more particularly hectorites modified with distearyldimethylammonium chloride.

In particular, the composition according to the invention comprises:
at least one non-starchy hydrophilic gelling agent chosen from crosslinked and/or neutralized 2-acrylamido-2-methylpropanesulfonic acid (AMPS®) copolymers, and more particularly copolymers of AMPS® and of hydroxyethyl acrylate, and
at least one lipophilic gelling agent chosen from hydrophobic silica aerogels,
at least one UV-screening agent,

and optionally an additional gelling agent chosen from particulate gelling agents, semicrystalline polymers, dextrin esters, polymers bearing hydrogen bonding, hydrocarbon-based block copolymers, and mixtures thereof, and in particular a modified clay and more particularly hectorites modified with distearyldimethylammonium chloride.

According to a third particular variant, the composition according to the invention comprises:
at least one non-starchy hydrophilic gelling agent chosen from crosslinked and/or neutralized 2-acrylamido-2-methylpropanesulfonic acid (AMPS®) copolymers, and more particularly copolymers of AMPS® and of hydroxyethyl acrylate, and
at least one lipophilic gelling agent chosen from organopolysiloxane elastomers,
at least one UV-screening agent,

and optionally an additional gelling agent chosen from particulate gelling agents, semicrystalline polymers, dextrin esters, polymers bearing hydrogen bonding, hydrocarbon-based block copolymers, and mixtures thereof, and in particular chosen from hydrophobic silica aerogels, modified clays, and mixtures thereof, in particular and more particularly hectorites modified with distearyldimethylammonium chloride, and mixtures thereof.

In particular, the composition according to the invention comprises:
at least one non-starchy hydrophilic gelling agent chosen from crosslinked and/or neutralized 2-acrylamido-2-methylpropanesulfonic acid (AMPS®) copolymers, and more particularly copolymers of AMPS® and of hydroxyethyl acrylate, and
at least one lipophilic gelling agent chosen from organopolysiloxane elastomers,

and optionally an additional gelling agent chosen from particulate gelling agents, semicrystalline polymers, dextrin esters, polymers bearing hydrogen bonding, hydrocarbon-based block copolymers, and mixtures thereof, and in particular hydrophobic silica aerogels; and
at least one UV-screening agent. Preferably, the total content of lipophilic gelling agent(s) is between 0.1% and 80% by weight expressed as solids, in particular from 0.2% to 60% by weight and preferably between 2% and 12% by weight relative to the total weight of the oily phase.

Even more particularly, said UV-screening agent is chosen from water-soluble organic UV-screening agents, liposoluble organic screening agents, and mixtures thereof, and more particularly liposoluble organic UV-screening agents.

Hydrophilic Gelling Agent/Lipophilic Gelling Agent System

As nonlimiting illustrations of hydrophilic gelling agent/lipophilic gelling agent systems that are most particularly suitable for use in the invention, mention may be made especially of the following systems:
crosslinked and/or neutralized copolymers of 2-acrylamido-2-methylpropanesulfonic acid/semicrystalline homopolymers or copolymers bearing at least one crystallizable side chain, said crystallizable chain being a side chain or in the backbone;
crosslinked and/or neutralized copolymers of 2-acrylamido-2-methylpropanesulfonic acid/hydrocarbon-based polyamides;
crosslinked and/or neutralized copolymers of 2-acrylamido-2-methylpropanesulfonic acid/hydrophobic silica aerogels;
and more particularly: crosslinked and/or neutralized copolymers of 2-acrylamido-2-methylpropanesulfonic acid/hectorites modified with a salt, preferably a C₁₀ to C₂₂ ammonium chloride;
modified or unmodified carboxyvinyl polymers/semicrystalline homopolymers or copolymers bearing at least one crystallizable side chain, said crystallizable chain being a side chain or in the backbone;
modified or unmodified carboxyvinyl polymers/hydrocarbon-based polyamides;
modified or unmodified carboxyvinyl polymers/hydrophobic silica aerogels;
and more particularly: modified or unmodified carboxyvinyl polymers/hectorites modified with a salt, preferably a C₁₀ to C₂₂ ammonium chloride.

According to a particularly preferred form, the composition according to the invention comprises:
at least one non-starchy hydrophilic gelling agent chosen from crosslinked and/or neutralized 2-acrylamido-2-methylpropanesulfonic acid (AMPS®) copolymers, and more particularly copolymers of AMPS® and of hydroxyethyl acrylate, and
at least one non-cellulose-based lipophilic gelling agent chosen from hectorites modified with a salt, preferably a C10 to C22 ammonium chloride, and more particularly hectorites modified with distearyldimethylammonium chloride,
at least one UV-screening agent.

According to a preferred variant, the gelling system comprises at least one hydrophobic silica aerogel.

As nonlimiting illustrations of hydrophilic gelling agent/lipophilic gelling agent systems that are most particularly suitable for use in the invention, mention may be made especially of the following systems:

crosslinked and/or neutralized copolymers of 2-acrylamido-2-methylpropanesulfonic acid/hydrophobic silica aerogels;

crosslinked and/or neutralized copolymers of 2-acrylamido-2-methylpropanesulfonic acid/hydrophobic silica aerogels and modified clays;

crosslinked and/or neutralized copolymers of 2-acrylamido-2-methylpropanesulfonic acid/hydrophobic silica aerogels and silicone elastomers;

crosslinked and/or neutralized copolymers of 2-acrylamido-2-methylpropanesulfonic acid/hydrophobic silica aerogels, modified clays and silicone elastomers.

According to a preferred variant, the gelling system comprises at least one organopolysiloxane elastomer.

As nonlimiting illustrations of hydrophilic gelling agent/lipophilic gelling agent systems that are most particularly suitable for use in the invention, mention may be made especially of the following systems:

crosslinked and/or neutralized copolymers of 2-acrylamido-2-methylpropanesulfonic acid/silicone elastomers;

crosslinked and/or neutralized copolymers of 2-acrylamido-2-methylpropanesulfonic acid/elastomers and hydrophobic silica aerogels;

crosslinked and/or neutralized copolymers of 2-acrylamido-2-methylpropanesulfonic acid/silicone elastomers and modified clays;

crosslinked and/or neutralized copolymers of 2-acrylamido-2-methylpropanesulfonic acid/silicone elastomers, hydrophobic silica aerogels and modified clays;

UV-Screening Agents

The compositions according to the invention contain at least one UV-screening agent. Preferably, the UV-screening agent that is suitable for use in the invention is chosen from water-soluble UV-screening agents, liposoluble UV-screening agents, insoluble UV-screening agents, and mixtures thereof. Among these UV-screening agents, a distinction can be made between water-soluble organic screening agents, liposoluble organic screening agents, insoluble organic screening agents and inorganic screening agents.

Preferentially, the UV-screening agent(s) are chosen from water-soluble UV-screening agents, liposoluble UV-screening agents, and mixtures thereof, and even more preferably chosen from water-soluble organic UV-screening agents and liposoluble organic screening agents, and mixtures thereof, and more particularly liposoluble organic UV-screening agents.

The term “water-soluble UV-screening agent” means any compound for screening out UV radiation, which can be fully dissolved or made miscible in molecular form in an aqueous phase or else which can be dissolved in colloidal form (for example in micellar form) in an aqueous phase.

The term “liposoluble UV-screening agent” means any compound for screening out UV radiation, which can be fully dissolved or made miscible in molecular form in a fatty phase or else which can be dissolved in colloidal form (for example in micellar form) in a fatty phase.

The term “insoluble UV-screening agent” means any compound for screening out UV radiation which has a solubility in water of less than 0.5% by weight and a solubility of less than 0.5% by weight in the majority of organic solvents such as liquid paraffin, fatty alcohol benzoates and fatty acid triglycerides, for example Miglyol 812® sold by the company Dynamit Nobel. This solubility, determined at 70° C., is defined as the amount of product in solution in the solvent at equilibrium with an excess of solid in suspension after returning to room temperature. It may be readily evaluated in the laboratory.

I/ Water-Soluble Organic UV-Screening Agents

A/ Water-Soluble Organic UVA-Screening Agents

The term “water-soluble organic UVA-screening agent” means any organic compound for screening out UVA radiation in the wavelength range 320 to 400 nm which can be fully dissolved or made miscible in molecular form in a liquid aqueous phase or else which can be dissolved in colloidal form (for example in micellar form) in an aqueous phase.

Among the water-soluble organic UVA-screening agents that may be used according to the present invention, mention may be made of:

benzene-1,4-bis(3-methylidene-10-camphorsulfonic acid) (INCI name: Terephthalylidene Dicamphor Sulfonic Acid) and the various salts thereof, described in particular in patent applications FR-A-2528420 and FR-A-2639347.

These screening agents correspond to general formula (I) below:

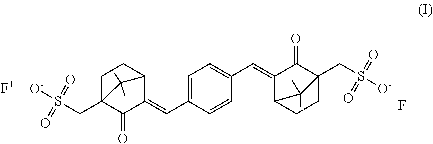

in which F denotes a hydrogen atom, an alkali metal or else a radical NH(R1)3+ in which the radicals R1, which may be identical or different, denote a hydrogen atom, a C1-C4 alkyl or hydroxyalkyl radical or else a group Mn+/n, Mn+ denoting a polyvalent metal cation in which n is equal to 2 or 3 or 4, Mn+ preferably denoting a metal cation chosen from Ca2+, Zn2+, Mg2+, Ba2+, Al3+ and Zr4+. It is clearly understood that the compounds of formula (I) above can give rise to the “cis-trans” isomer around one or more double bond(s) and that all the isomers are within the context of the present invention.

Among the water-soluble organic UVA-screening agents that can be used according to the present invention, mention may also be made of compounds comprising at least two benzazolyl groups bearing sulfonic groups, such as those described in patent application EP-A-0 669 323. They are described and prepared according to the syntheses indicated in patent U.S. Pat. No. 2,463,264 and also in patent application EP-A-0 669 323.

The compounds comprising at least two benzazolyl groups in accordance with the invention correspond to general formula (II) below:

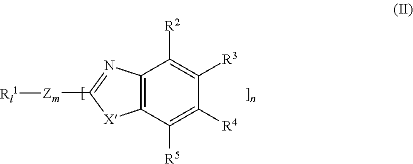

in which:
Z represents an organic residue of valency (1+n) comprising one or more double bonds placed such that it completes the system of double bonds of at least two benzazolyl groups as defined inside the square brackets so as to form a totally conjugated assembly;
X′ denotes S, O or NR6;
R1 denotes hydrogen, C1-C18 alkyl, C1-C4 alkoxy, a C5-C15 aryl, a C2-C18 acyloxy, SO3Y or COOY;
the radicals R2, R3, R4 and R5, which may be identical or different, denote a nitro group or a radical R1;
R6 denotes hydrogen, a C1-C4 alkyl or a C1-C4 hydroxyalkyl;
Y denotes hydrogen, Li, Na, K, NH4, 1/2Ca, 1/2Mg, 1/3Al or a cation resulting from the neutralization of a free acid group with an organic nitrogenous base;
m is 0 or 1;
n is a number from 2 to 6;
1 is a number from 1 to 4;
with the proviso that 1+n does not exceed the value 6.

Among these compounds, preference is given to those for which the group Z is chosen from the group made up of:

(a) an olefin linear aliphatic C2-C6 hydrocarbon-based radical which may be interrupted with a C5-C12 aryl group or a C4-C10 heteroaryl group, in particular chosen from the following groups:

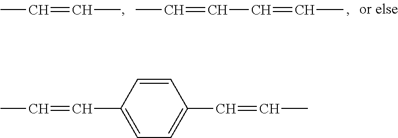

(b) a C₅-C₁₅ aryl group which may be interrupted with an olefin linear aliphatic C₂-C₆ hydrocarbon-based radical, in particular chosen from the following groups:

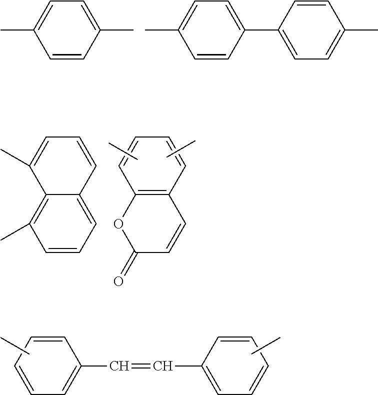

(c) a C₃-C₁₀ heteroaryl residue, in particular chosen from the following groups:

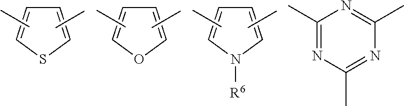

in which R⁶ has the same meaning as that indicated above; said radicals Z as defined in paragraphs (a), (b) and (c) possibly being substituted with C₁-C₆ alkyl, C₁-C₆ alkoxy, phenoxy, hydroxyl, methylenedioxy or amino radicals optionally substituted with one or two C₁-C₅ alkyl radicals.

Preferably, the compounds of formula (II) comprise, per molecule, 1, 3 or 4 SO₃Y groups.

As examples of compounds of formula (II) that may be used, mention may be made of the compounds of formulae (a) to (j) having the following structure, and also the salts thereof:

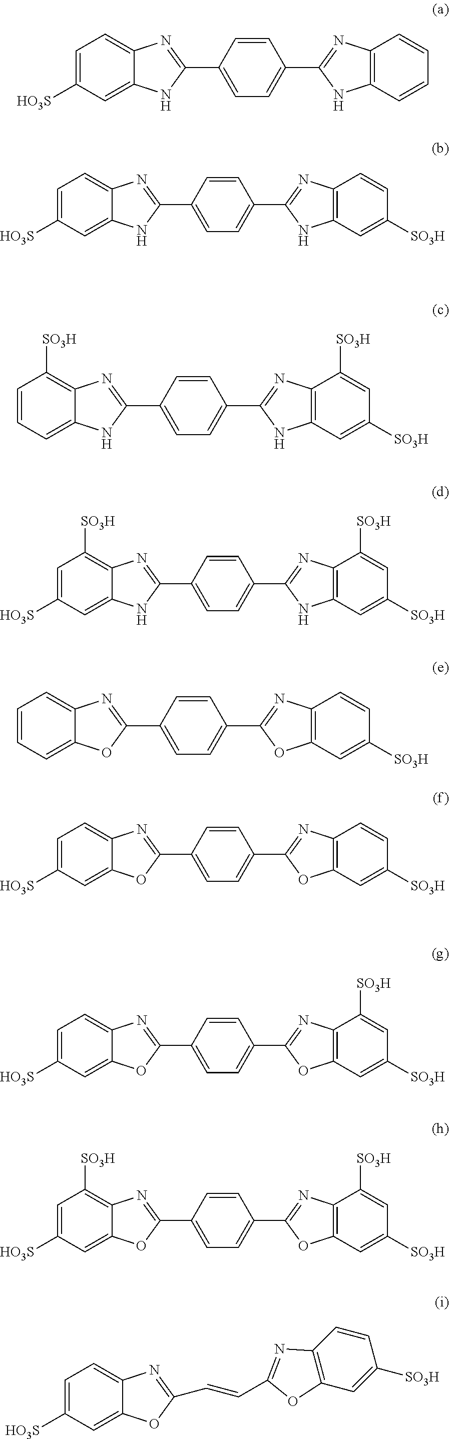

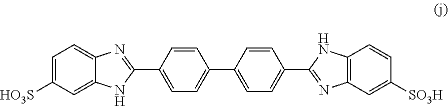 [SMILES: O=S(=O)(O)c1ccc2[nH]c(-c3ccc(-c4ccc(-c5nc6ccc(S(=O)(=O)O)cc6[nH]5)cc4)cc3)nc2c1]

Among all these compounds, preference will most particularly be given to 1,4-bis-benzimidazolyl-phenylene-3,3′,5,5′-tetrasulfonic acid (INCI name: Disodium Phenyl Dibenzimidazole Tetrasulfonate) (compound (d′)) or a salt thereof, having the following structure, sold especially under the name Neoheliopan AP® by the company Symrise:

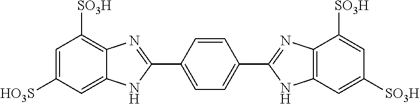 [SMILES: O=S(=O)(O)c1cc(S(=O)(=O)O)c2nc(-c3ccc(-c4nc5c(S(=O)(=O)O)cc(S(=O)(=O)O)cc5[nH]4)cc3)[nH]c2c1]

Among the water-soluble organic UVA-screening agents that may be used according to the present invention, mention may also be made of benzophenone compounds comprising at least one sulfonic acid function, for instance the following compounds:

Benzophenone-4, sold by the company BASF under the name Uvinul MS40®:

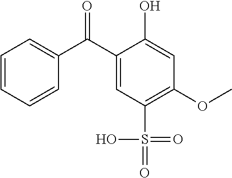 [SMILES: COc1cc(O)c(C(=O)c2ccccc2)cc1S(=O)(=O)O]

Benzophenone-5 having the structure

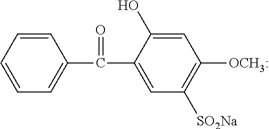

Benzophenone-9, sold by the company BASF under the name Uvinul DS49®:

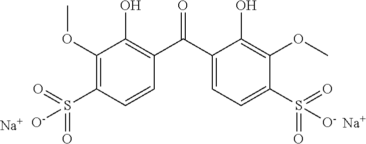 [SMILES: [Na+] ; COc1c(S(=O)(=O)[O-])ccc(C(=O)c2ccc(S(=O)(=O)[O-])c(OC)c2O)c1O ; [Na+]]

Among the water-soluble organic UVA-screening agents, use will more particularly be made of benzene-1,4-bis(3-methylidene-10-camphorsulfonic acid) and the various salts thereof (INCI name: Terephthalylidene Dicamphor Sulfonic Acid) manufactured especially by the company Chimex under the trade name Mexoryl SX®.

The water-soluble organic UVA-screening agent(s) in accordance with the invention are preferably present in the compositions according to the invention at an active material concentration ranging from 0.01% to 30% and preferably from 0.1% to 15% by weight relative to the total weight of the composition.

B/ Water-Soluble Organic UVB-Screening Agents

The term “water-soluble organic UVB-screening agent” means any organic compound for screening out UVB radiation in the wavelength range 280 to 320 nm which can be fully dissolved or made miscible in molecular form in a liquid aqueous phase or else which can be dissolved in colloidal form (for example in micellar form) in an aqueous phase.

The water-soluble organic UVB-screening agents are especially chosen from:

water-soluble cinnamic derivatives such as ferulic acid or 3-methoxy-4-hydroxycinnamic acid,

water-soluble benzylidenecamphor compounds;

water-soluble phenylbenzimidazole compounds;

water-soluble p-aminobenzoic (PABA) compounds;

water-soluble salicylic compounds;

and mixtures thereof.

As examples of water-soluble organic UVB-screening agents, mention may be made of those denoted hereinbelow under their INCI name:

para-Aminobenzoic Compounds:

PABA,

PEG-25 PABA, sold especially under the name Uvinul P 25® by BASF.

Salicylic Compounds:

Dipropylene glycol salicylate, sold especially under the name Dipsal® by Scher,

TEA salicylate, sold especially under the name Neo Heliopan TS® by Symrise,

Benzylidenecamphor Compounds:

Benzylidenecamphorsulfonic acid, sold especially under the name Mexoryl SL® by Chimex,

Camphor benzalkonium methosulfate, sold especially under the name Mexoryl SO® by Chimex.

Phenylbenzimidazole Compounds:

Phenylbenzimidazolesulfonic acid, sold in particular under the trade name Eusolex 232® by Merck.

Use will more particularly be made of the screening agent phenylbenzimidazolesulfonic acid, sold especially under the trade name Eusolex 232® by Merck.

The water-soluble organic UVB-screening agent(s) in accordance with the invention are preferably present in the compositions according to the invention at an active material concentration ranging from 0.01% to 30% and preferably from 0.1% to 15% by weight relative to the total weight of the composition.

II/ Liposoluble Organic UV-Screening Agents

The term “liposoluble organic UVB-screening agent” means any organic compound for screening out UVB radiation in the wavelength range 280 to 320 nm which can be fully dissolved or made miscible in molecular form in a fatty phase or else which can be dissolved in colloidal form (for example in micellar form) in a fatty phase. Among the liposoluble organic UV-screening agents, some of them are liquid at room temperature.

The liposoluble organic UV-screening agents are chosen especially from cinnamic derivatives; anthranilates; salicylic derivatives, dibenzoylmethane derivatives, camphor derivatives; benzophenone derivatives; β,β-diphenylacrylate derivatives; triazine derivatives; benzotriazole derivatives; benzalmalonate derivatives, in particular those cited in patent U.S. Pat. No. 5,624,663; imidazolines; p-aminobenzoic (PABA) derivatives; benzoxazole derivatives, as described in patent applications EP 0 832 642, EP 1 027 883, EP 1 300 137 and DE 101 62 844; screening polymers and screening silicones, such as those described in particular in application WO 93/04665; α-alkylstyrene-based dimers, such as those described in patent application DE 198 55 649; 4,4-diarylbutadienes as described in applications EP 0 967 200, DE 197 46 654, DE 197 55 649, EP-A-1 008 586, EP 1 133 980 and EP 133 981; merocyanine derivatives, merocyanines as described in patent U.S. Pat. No. 4,195,999, application WO 2004/006878, applications WO 2008/090066, WO 2011/113718 and WO 2009/027258, and the documents IP COM Journal No. 000179675D published on 23 Feb. 2009, IP COM Journal No. 000182396D published on 29 Apr. 2009, IP COM Journal No. 000189542D published on 12 Nov. 2009 and IP COM Journal No. IPCOM000011179D published on 04/03/2004, and mixtures thereof.

As examples of liposoluble organic UV-screening agents, mention may be made of those denoted hereinbelow under their INCI name:

Dibenzoylmethane Derivative:

Butylmethoxydibenzoylmethane or avobenzone sold in particular under the trade name Parsol 1789 by the company DSM Nutritional Products;

para-Aminobenzoic Acid Derivatives:

Ethyl PABA,

Ethyl dihydroxypropyl PABA,

Ethylhexyl dimethyl PABA sold in particular under the name Escalol 507 by ISP;

Salicylic Derivatives:

Homosalate sold in particular under the name Eusolex HMS by Rona/EM Industries,

Ethylhexyl salicylate sold in particular under the name Neo Heliopan OS by Symrise;

Cinnamic Derivatives:

Ethylhexyl Methoxycinnamate, sold especially under the trade name Parsol MCX by DSM Nutritional Products,

Isopropyl methoxycinnamate,

Isoamyl methoxycinnamate sold in particular under the trade name Neo Heliopan E 1000 by Symrise,

Cinoxate,

Diisopropyl methyl cinnamate;

β,β-Diphenylacrylate Derivatives:

Octocrylene sold especially under the trade name Uvinul N539 by BASF,

Etocrylene sold in particular under the trade name Uvinul N35 by BASF;

Benzophenone Derivatives:

Benzophenone-1 sold in particular under the trade name Uvinul 400 by BASF,

Benzophenone-2 sold in particular under the trade name Uvinul D50 by BASF,

Benzophenone-3 or oxybenzone sold in particular under the trade name Uvinul M40 by BASF,

Benzophenone-6 sold in particular under the trade name Helisorb 11 by Norquay,

Benzophenone-8 sold in particular under the trade name Spectra-Sorb UV-24 by American Cyanamid,

Benzophenone-12,

n-hexyl 2-(4-diethylamino-2-hydroxybenzoyl)benzoate sold in particular under the trade name Uvinul A+, such as Uvinul A+Granular, or in the form of a mixture with octyl methoxycinnamate in particular under the trade name Uvinul A+B by BASF;

Benzylidene Camphor Derivatives:

3-Benzylidenecamphor sold in particular under the name Mexoryl SD by Chimex,

4-Methylbenzylidenecamphor sold in particular under the name Eusolex 6300 by Merck,

Polyacrylamidomethylbenzylidenecamphor sold in particular under the name Mexoryl SW by Chimex;

Phenylbenzotriazole Derivatives:

Drometrizole Trisiloxane, sold under the name Silatrizole by Rhodia Chimie;

Triazine Derivatives:

Bis(ethylhexyloxyphenol)methoxyphenyltriazine sold in particular under the trade name Tinosorb S by BASF,

Ethylhexyl triazone sold in particular under the trade name Uvinul T150 by BASF,

Diethylhexyl butamido triazone sold in particular under the trade name Uvasorb HEB by Sigma 3V,
Silicone triazines substituted with two aminobenzoate groups, as described in patent EP 0 841 341, in particular 2,4-bis(n-butyl 4′-aminobenzalmalonate)-6-[(3-{1,3,3,3-tetramethyl-1-[(trimethylsilyl)oxy]disiloxanyl}propyl)amino]-s-triazine;

Anthranilic Derivatives:

Menthyl anthranilate sold in particular under the trade name Neo Heliopan MA by Symrise;

Imidazoline Derivatives:

Ethylhexyl dimethoxybenzylidene dioxoimidazoline propionate;

Benzalmalonate Derivatives:

Dineopentyl 4′-methoxybenzalmalonate,

Polyorganosiloxane containing benzalmalonate functions, for instance Polysilicone-15 sold in particular under the trade name Parsol SLX by DSM;

4,4-Diarylbutadiene Derivatives:

1,1-Dicarboxy(2,2′-dimethylpropyl)-4,4-diphenylbutadiene;

Benzoxazole Derivatives:

2,4-Bis[5-(1,-dimethylpropyl)benzoxazol-2-yl-(4-phenyl)imino]-6-(2-ethylhexyl)imino-1,3,5-triazine sold in particular under the name Uvasorb K2A by Sigma 3V,

and mixtures thereof;

Lipophilic Merocyanine Derivatives:
Octyl 5-N,N-diethylamino-2-phenylsulfonyl-2,4-pentadienoate;

and mixtures thereof;

The preferential liposoluble organic screening agents are chosen from:

Butyl methoxydibenzoylmethane

Ethylhexyl methoxycinnamate

Ethylhexyl salicylate,

Homosalate,

Butyl methoxydibenzoylmethane

octocrylene,

Benzophenone-3,

n-Hexyl 2-(4-diethylamino-2-hydroxybenzoyl)benzoate,

4-Methylbenzylidene camphor,

bis-Ethylhexyloxyphenol methoxyphenyl triazine,

Ethylhexyl triazone,

Diethylhexyl butamido triazone,

2,4,6-tris(dineopentyl 4′-aminobenzalmalonate)-s-triazine,

2,4,6-tris(diisobutyl 4′-aminobenzalmalonate)-s-triazine,

2,4-bis(dineopentyl 4′-aminobenzalmalonate)-6-(n-butyl 4′-aminobenzoate)-s-triazine,

Drometrizole trisiloxane,

Polysilicone-15,

1,1-Dicarboxy(2,2′-dimethylpropyl)-4,4-diphenylbutadiene,

2,4-bis[5-(1-Dimethylpropyl)benzoxazol-2-yl-(4-phenyl)imino]-6-(2-ethylhexyl)imino-1,3,5-triazine,

and mixtures thereof.

The preferential liposoluble organic screening agents are more particularly selected from:

Butyl methoxydibenzoylmethane

octocrylene,

Ethylhexyl salicylate,

n-Hexyl 2-(4-diethylamino-2-hydroxybenzoyl)benzoate,

bis-Ethylhexyloxyphenol methoxyphenyl triazine,

Ethylhexyl triazone,

Diethylhexyl butamido triazone,

Drometrizole trisiloxane, and mixtures thereof.

The liposoluble organic UV-screening agent(s) are preferably present in the compositions according to the invention in a content ranging from 0.1% to 50% by weight and in particular from 0.5% to 30% by weight relative to the total weight of the composition.

III/ Insoluble Organic And Inorganic UV-Screening Agents

A/ Insoluble Organic UV-Screening Agents

The insoluble organic UV-screening agents according to the invention preferably have a mean particle size which ranges from 0.01 to 5 μm and more preferentially from 0.01 to 2 μm and more particularly from 0.020 to 2 μm.

The mean particle diameter is measured using a particle size distribution analyzer of the Culter N4 PLUS type manufactured by Beckman Coulter Inc.

The insoluble organic screening agents according to the invention can be brought to the desired particulate form by any ad hoc means, especially such as dry milling or milling in a solvent medium, sieving, atomization, micronization or spraying.

The insoluble organic screening agents according to the invention in micronized form may in particular be obtained by means of a process of milling an insoluble organic UV-screening agent in the form of particles of coarse size in the presence of an appropriate surfactant making it possible to improve the dispersion of the resulting particles in the cosmetic formulations.

An example of a process for micronization of insoluble organic screening agents is described in applications GB-A-2 303 549 and EP-A-893119. The milling apparatus used according to these documents may be a jet, ball, vibration or hammer mill and preferably a high speed stirring mill or an impact mill and more particularly a rotating ball mill, a vibrating mill, a tube mill or a rod mill.

According to this particular process, use is made, as surfactants for milling said screening agents, of alkylpolyglucosides having the structure CₙH₂ₙ₊₁ O(C₆H₁₀O₅)ₓH in which n is an integer from 8 to 16 and x is the mean degree of polymerization of the unit (C₆H₁₀O₅) and ranges from 1.4 to 1.6. They may be chosen from C₁-C₁₂ esters of a compound having the structure CₙH₂ₙ₊₁ O(C₆H₁₀O₅)ₓH and more particularly an ester obtained by reacting a C₁-C₁₂ carboxylic acid, such as formic, acetic, propionic, butyric, sulfosuccinic, citric or tartaric acid, with one or more free OH functions on the glucoside unit (C₆H₁₀O₅). Decylglucoside may in particular be mentioned as alkylpolyglucoside.

Said surfactants are generally used at a concentration ranging from 1% to 50% by weight and more preferentially from 5% to 40% by weight, relative to the insoluble screening agent in its micronized form.

The insoluble organic UV-screening agents in accordance with the invention may be chosen in particular from organic UV-screening agents of the oxalanilide type, of the triazine type, of the benzotriazole type; of the vinylamide type; of the cinnamide type; of the type comprising one or more groups which are benzazole and/or benzofuran, benzothiophene or of the indole type; of the aryl vinylene ketone type; of the phenylene bis-benzoxazinone derivative type; of the amide, sulfonamide or acrylonitrile carbamate derivative type, or mixtures thereof.

For the purpose for which it is used in the present invention, the term “benzazole” encompasses at the same time benzothiazoles, benzoxazoles and benzimidazoles.

A/ Oxalanides

Among the UV-screening agents of the oxalanilide type in accordance with the invention, mention may be made of those corresponding to the structure:

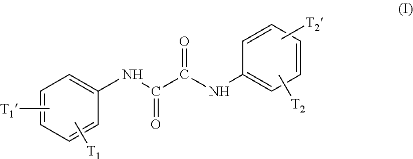

in which T₁, T₁, T₂ and T′₂ denote, identically or differently, a C₁ to C₈ alkyl radical or a C₁ to C₈ alkoxy radical. These compounds are described in patent application WO 95/22959.

By way of example, mention may be made of the commercial products Tinuvin 315® and Tinuvin 312® sold by the company BASF and respectively having the structure:

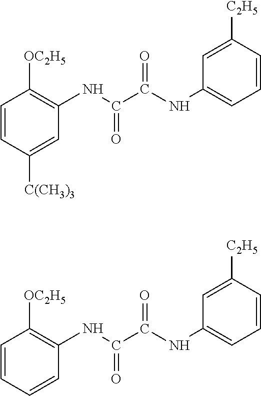 [SMILES: CCOc1ccc(C(C)(C)C)cc1NC(=O)C(=O)Nc1cccc(CC)c1 ; CCOc1ccccc1NC(=O)C(=O)Nc1cccc(CC)c1]

B/ Triazines

Among the insoluble UV-screening agents of the triazine type in accordance with the invention, mention may also be made of those corresponding to formula (II) below:

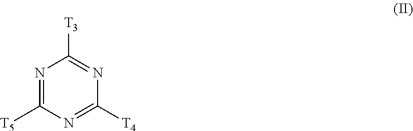

in which T₃, T₄ and T₅, independently, are phenyl, phenoxy or pyrrolo, in which the phenyl, phenoxy and pyrrolo are unsubstituted or substituted with one, two or three substituents chosen from OH, C₁-C₁₈ alkyl or C₁-C₁₈ alkoxy, C₁-C₁₈ carboxyalkyl, C₅-C₈cycloalkyl, a methylbenzylidenecamphor group, a —(CH═CH)ₙ(CO)—OT₆ group, with T₆ being either C₁-C₁₈alkyl or cinnamyl.

These compounds are described in WO 97/03642, GB 2286774, EP-743309, WO 98/22447 and GB 2319523.

Among the UV-screening agents of the triazine type in accordance with the invention, mention may also be made of insoluble derivatives of s-triazine bearing benzalmalonate and/or phenyl cyanoacrylate groups, such as those described in application EP-A-0790243 (which is an integral part of the content of the description).

Among these insoluble UV-screening agents of the triazine type, mention will more particularly be made of the following compounds:
2,4,6-tris(diethyl 4′-aminobenzalmalonate)-s-triazine,
2,4,6-tris(diisopropyl 4′-aminobenzalmalonate)-s-triazine,
2,4,6-tris(dimethyl 4′-aminobenzalmalonate)-s-triazine,
2,4,6-tris(ethyl α-cyano-4-aminocinnamate)-s-triazine.

Among the UV-screening agents of the triazine type in accordance with the invention, mention may also be made of insoluble derivatives of s-triazine bearing benzotriazole and/or benzothiazole groups, such as those described in application WO 98/25922 (which forms an integral part of the content of the description).

Among these compounds, mention may more particularly be made of:
2,4,6-tris[(3′-benzotriazol-2-yl-2′-hydroxy-5′-methyl)phenylamino]-s-triazine,
2,4,6-tris[(3′-benzotriazol-2-yl-2′-hydroxy-5′-tert-octyl)phenylamino]-s-triazine.

Mention may also be made of the symmetrical triazine screening agents described in patent U.S. Pat. No. 6,225,467, patent application WO 2004/085412 (see compounds 6 and 9) or the document “Symmetrical Triazine Derivatives” IP.COM Journal, IP.COM INC West Henrietta, N.Y., US (20 Sep. 2004), in particular 2,4,6-tris(biphenyl)-1,3,5-triazines (in particular 2,4,6-tris(biphenyl-4-yl)-1,3,5-triazine) and 2,4,6-tris(terphenyl)-1,3,5-triazine which is also mentioned in Beiersdorf patent applications WO 06/035 000, WO 06/034 982, WO 06/034 991, WO 06/035 007, WO 2006/034 992 and WO 2006/034 985.

C/ Benzotriazoles

Among the insoluble organic UV-screening agents of the benzotriazole type in accordance with the invention, mention may be made of those of formula (III) below, as described in application WO 95/22959 (which forms an integral part of the content of the description):

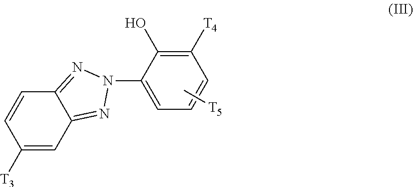

in which T₇ denotes a hydrogen atom or a C₁ to C₁₈ alkyl radical; and T₈ and T₉, which may be identical or different, denote a C₁ to C₁₈ alkyl radical optionally substituted with a phenyl.

As examples of compounds of formula (III), mention may be made of the commercial products Tinuvin 328, 320, 234 and 350 from the company BASF, having the structure below:

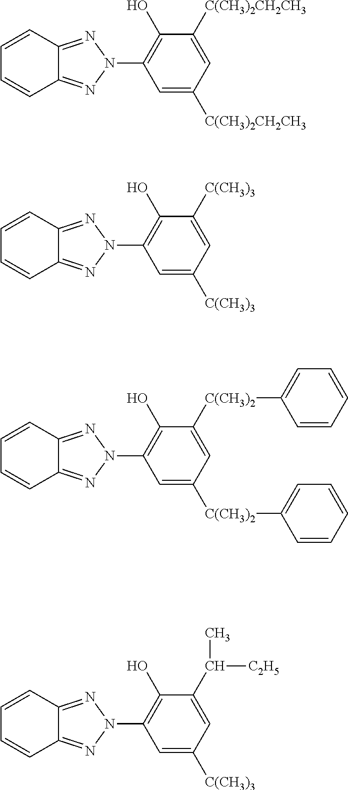

Among the insoluble organic UV-screening agents of the benzotriazole type in accordance with the invention, mention may be made of the compounds as described in patents U.S. Pat. No. 5,687,521, U.S. Pat. No. 5,373,037 and U.S. Pat. No. 5,362,881 and in particular [2,4′-dihydroxy-3-(2H-benzotriazol-2-yl)-5-(1,1,3,3-tetramethylbutyl)-2′-n-octoxy-5′-benzoyl]diphenylmethane sold especially under the name Mixxim PB30® by the company Fairmount Chemical, of structure:

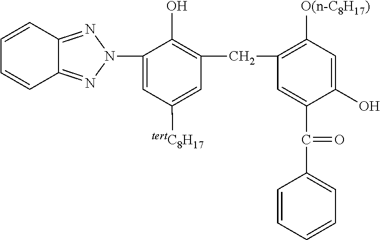

Among the insoluble organic UV-screening agents of the benzotriazole type in accordance with the invention, mention may be made of the methylenebis(hydroxyphenylbenzotriazole) derivatives having the structure below:

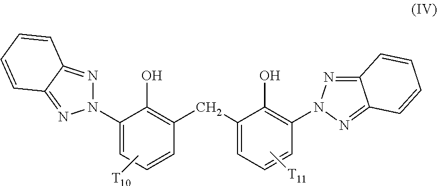

in which the radicals T₁₀ and T₁₁, which may be identical or different, denote a C₁ to C₁₈ alkyl radical which may be substituted with one or more radicals chosen from C₁-C₄ alkyl, C₅-C₁₂ cycloalkyl or an aryl residue. These compounds are known per se and described in applications 5 U.S. Pat. No. 5,237,071, U.S. Pat. no. 5,166,355, GB-A-2 303 549, DE 197 26 184 and EP-A-893 119 (which are an integral part of the description).

In formula (I) defined above: the C₁-C₁₈ alkyl groups may be linear or branched and are, for example, methyl, ethyl, n-propyl, isopropyl, n-butyl, isobutyl, tert-butyl, tert-octyl, n-amyl, n-hexyl, n-heptyl, n-octyl, isooctyl, n-nonyl, n-decyl, n-undecyl, n-dodecyl, tetradecyl, hexadecyl or octadecyl; the C₅-C₁₂ cycloalkyl groups are, for example, cyclopentyl, cyclohexyl or cyclooctyl; the aryl groups are, for example, phenyl or benzyl.

Among the compounds of formula (IV), mention may be made of those having the structure below:

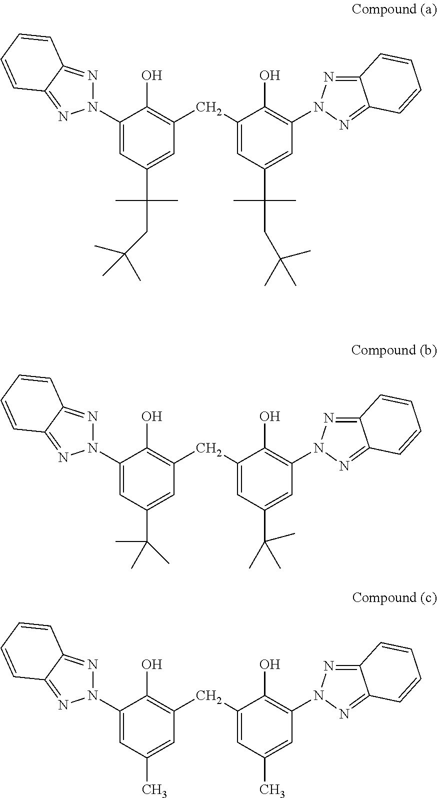 [SMILES: CC(C)(C)CC(C)(C)c1cc(Cc2cc(C(C)(C)CC(C)(C)C)cc(-n3nc4ccccc4n3)c2O)c(O)c(-n2nc3ccccc3n2)c1 ; CC(C)(C)c1cc(Cc2cc(C(C)(C)C)cc(-n3nc4ccccc4n3)c2O)c(O)c(-n2nc3ccccc3n2)c1 ; Cc1cc(Cc2cc(C)cc(-n3nc4ccccc4n3)c2O)c(O)c(-n2nc3ccccc3n2)c1]

Compound (a) of nomenclature 2,2′-methylenebis[6-(2H-benzotriazol-2-yl)-4-(1,1,3,3-tetramethylbutyl)phenol] is sold especially under the trade name Mixxim BB/200® by the company Fairmount Chemical.

Compound (c) of nomenclature 2,2′-methylenebis[6-(2H-benzotriazol-2-yl)-4-(methyl)phenol] is sold especially in solid form under the trade name Mixxim BB/200® by the company Fairmount Chemical.

D/ Vinyl Amides

Among the insoluble organic screening agents of the vinylamide type, mention may be made for example of the compounds having the formula below which are described in application WO 95/22959 (which is an integral part of the content of the description):

T₁₂-(Y)r-C(═O)—C(T₁₃)=C(T₁₄)-N(T₁₅)(T₁₆) (V)

in which T₁₂ is a C₁ to C₁₈, preferably C₁ to C₅, alkyl radical or a phenyl group which is optionally substituted with one, two or three radicals chosen from OH, C₁ to C₁₈ alkyl, C₁ to C₈ alkoxy, or a —C(═O)—OT₁₇ group where T₁₇ is a C₁ to C₁₈ alkyl; T₁₃, T₁₄, T₁₅ and T₁₆, which may be identical or different, denote a C₁ to C₁₈, preferably C₁ to C₅, alkyl radical or a hydrogen atom; Y is N or O and r is 0 or 1.

Among these compounds, mention will be made especially of:
4-octylamino-3-penten-2-one;
ethyl 3-octylamino-2-butenoate;
3-octylamino-1-phenyl-2-buten-1-one;
3-dodecylamino-1-phenyl-2-buten-1-one.

E/ Cinnamamides

Among the insoluble organic screening agents of the cinnamamide type in accordance with the invention, mention may also be made of the compounds as described in patent application WO 95/22959 (which forms an integral part of the content of the description) and which correspond to the structure below:

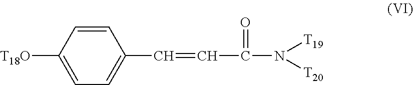

in which OT₁₈ is a hydroxyl or C₁ to C₄ alkoxy radical, preferably methoxy or ethoxy; T₁₉ is hydrogen or C₁ to C₄ alkyl, preferably methyl or ethyl; T₂₀ is a —(CONH)ₛ— phenyl group where s is 0 or 1 and the phenyl group may be substituted with one, two or three groups chosen from OH, C₁ to C₁₈ alkyl, C₁ to C₈ alkoxy, or a —C(═O)—OT₂₁ group where T₂₁ is a C₁ to C₁₈ alkyl and more preferentially T₂₁ is a phenyl, 4-methoxyphenyl or phenylaminocarbonyl group.

Mention may also be made of cinnamamide dimers such as those described in patent U.S. Pat. No. 5,888,481, for instance the compound having the structure:

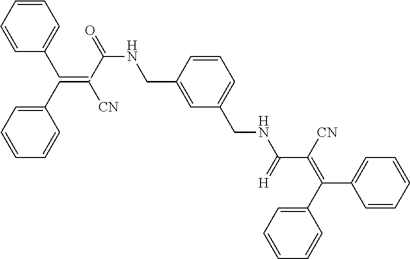

F/ Benzazoles

Among the insoluble organic screening agents of the benzazole type, mention may be made of those corresponding to one of the formulae below:

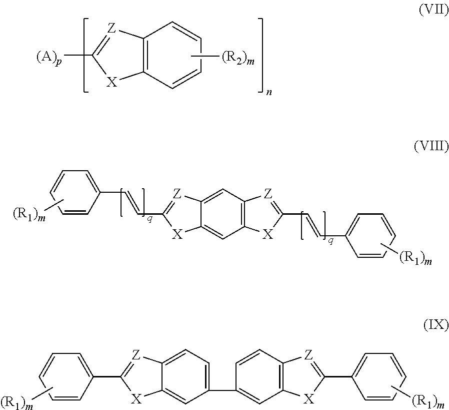

in which each of the symbols X independently represents an oxygen or sulfur atom or a group NR₂, each of the symbols Z independently represents a nitrogen atom or a CH group,

each of the symbols R₁ independently represents an OH group, a halogen atom, a linear or branched C₁-C₈ alkyl group, optionally containing a silicon atom, or a linear or branched C₁-C₈ alkoxy group,

each of the numbers m is independently 0, 1 or 2,

n represents an integer between 1 and 4 inclusive,

p is equal to 0 or 1,

each of the numbers q is independently equal to 0 or 1,

each of the symbols R2 independently represents a hydrogen atom, or a benzyl or linear or branched C₁-C₈ alkyl group, optionally containing a silicon atom,

A represents a radical of valency n chosen from those of formulae:

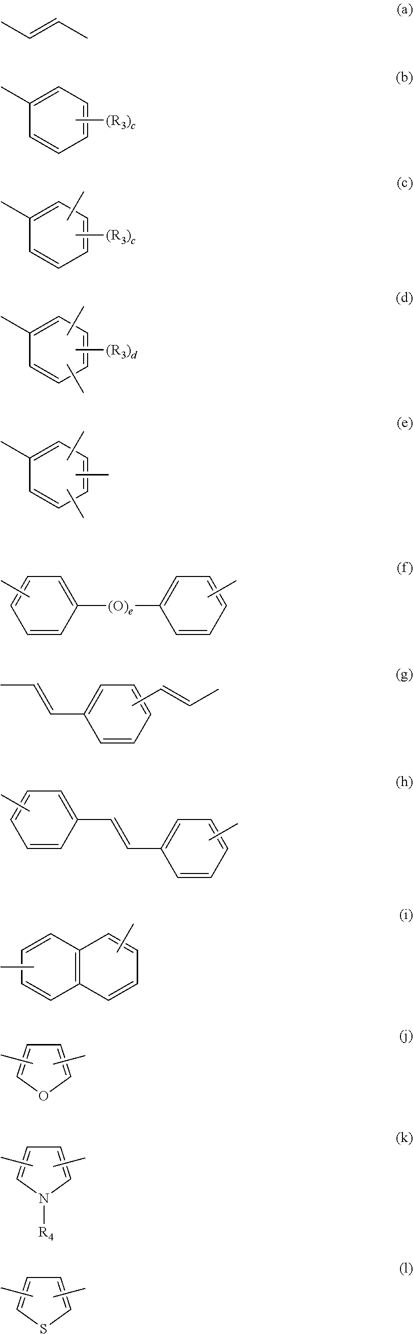

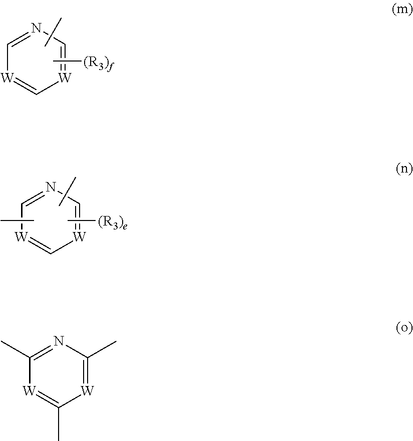

in which each of the symbols R₃ independently represents a halogen atom or a linear or branched C₁-C₄ alkyl or alkoxy group or a hydroxyl group, and R₄ represents a hydrogen atom or a linear or branched C₁-C₄ alkyl group, c=0-4, d=0-3, e=0 or 1, and f=0-2.

These compounds are especially described in patents DE 676 103 and CH 350 763, patent U.S. Pat. No. 5,501,850, patent U.S. Pat. No. 5,961,960, patent application EP0669323, patent U.S. Pat. No. 5,518,713, patent U.S. Pat. No. 2,463,264, the article J. Am. Chem. Soc., 79, 5706-5708, 1957, the article J. Am. Chem. Soc., 82, 609-5 611, 1960, patent application EP0921126, and patent application EP712855.

As examples of preferred compounds of formula (VII) of the 2-arylbenzazole family, mention may be made of 2-benzoxazol-2-yl-4-methylphenol, 2-(1H-benzimidazol-2-yl)-4-methoxyphenol or 2-benzothiazol-2-ylphenol, these compounds possibly being prepared, for example, according to the processes described in patent CH 350 763.

As examples of preferred compounds of formula (VII) of the benzimidazolylbenzazole family, mention will be made of 2,2′-bis-benzimidazole, 5,5′,6,6′-tetramethyl-2,2′-bis-benzimidazole, 5,5′-dimethyl-2,2′-bis-benzimidazole, 6-methoxy-2,2′-bis-benzimidazole, 2-(1H-benzimidazol-2-yl)benzothiazole, 2-(1H-benzimidazol-2-yl)benzoxazole and N,N′-dimethyl-2,2′-bis-benzimidazole, these compounds possibly being prepared according to the procedures described in patents U.S. Pat. No. 5,961,960 and U.S. Pat. No. 2,463,264.

As examples of preferred compounds of formula (VII) of the phenylenebenzazole family, mention will be made of 1,4-phenylene-bis-(2-benzoxazolyl), 1,4-phenylene-bis-(2-benzimidazolyl), 1,3-phenylene-bis-(2-benzoxazolyl), 1,2-phenylene-bis-(2-benzoxazolyl), 1,2-phenylene-bis-(benzimidazolyl), 1,4-phenylene-bis-(N-2-ethylhexyl-2-benzimidazolyl) and 1,4-phenylene-bis-(N-trimethylsilylmethyl-2-benzimidazolyl), these compounds possibly being prepared according to the procedures described in patent US 2 463 264 and in the publications J. Am. Chem. Soc., 82, 609 (1960) and J. Am. Chem. Soc., 79, 5706-5708 (1957).

As examples of preferred compounds of formula (VII) of the benzofuranylbenzoxazole family, mention will be made of 2-(2-benzofuranyl)benzoxazole, 2-(benzofuranyl)-5-methylbenzoxazole and 2-(3-methyl-2-benzofuranyl)benzoxazole, these compounds possibly being prepared according to the procedures described in patent U.S. Pat. No. 5,518,713.

As preferred compounds of formula (VIII), mention may be made, for example, of 2,6-diphenyl-1,7-dihydrobenzo[1,2-d;4,5-d′]diimidazole corresponding to the formula:

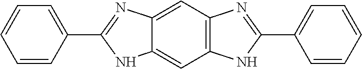 [SMILES: c1ccc(-c2nc3cc4nc(-c5ccccc5)[nH]c4cc3[nH]2)cc1]

or 2,6-distyryl-1,7-dihydrobenzo[1,2-d; 4,5-d′]diimidazole or else 2,6-di(p-tert-butylstyryl)-1,7-dihydrobenzo[1,2-d; 4,5-d′]diimidazole, which may be prepared according to patent application EP 0 669 323.

As preferred compound of formula (IX), mention may be made of 5,5′-bis-[(phenyl-2)-benzimidazole] having the formula:

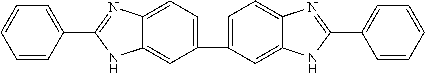 [SMILES: c1ccc(-c2nc3ccc(-c4ccc5nc(-c6ccccc6)[nH]c5c4)cc3[nH]2)cc1]

the preparation of which is described in J. Chim. Phys., 64, 1602 (1967).

Among these insoluble organic compounds which screen out UV radiation, preference is given most particularly to 2-(1H-benzimidazol-2-yl)benzoxazole, 5 ole, 6-methoxy-2,2′-bis-benzimidazole, 2-(1H-benzimidazol-2-yl)benzothiazole, 1,4-phenylenebis-(2-benzoxazolyl), 1,4-phenylene-bis-(2-benzimidazolyl), 1,3-phenylenebis-(2-benzoxazolyl), 1,2-phenylene-bis-(2-benzoxazolyl), 1,2-phenylenebis-(2-benzimidazolyl) and 1,4-phenylene-bis-(N-trimethylsilylmethyl-2-benzimidazolyl).

G/ Aryl Vinylene Ketones

Among the insoluble organic screening agents of the aryl vinylene ketone type, mention may be made of those corresponding to one of formulae (X) and (XI) below:

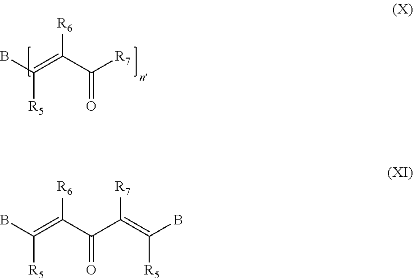

in which:

n′=1 or 2,

B, in formula (X) when n′=1 or in formula (XI), is an aryl radical chosen from formulae (a′) to (d′) below, or, in formula (X) when n′=2, is a radical chosen from formulae (e′) to (h′) below:

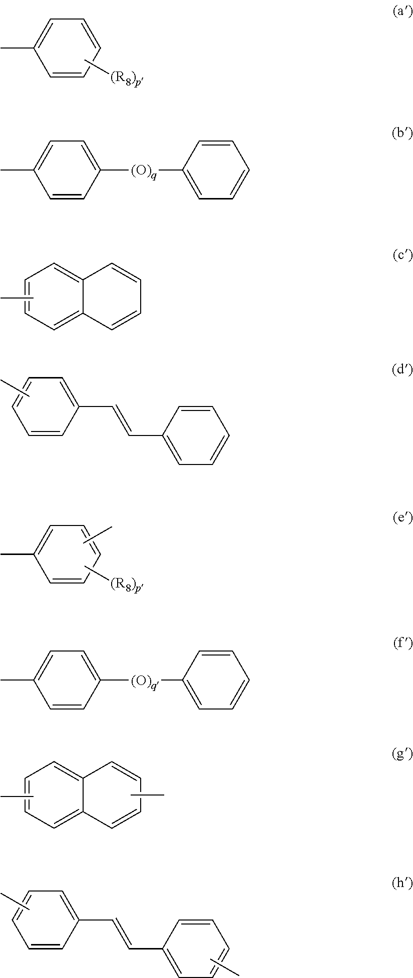

in which:

each of the symbols R₈ independently represents an OH group, a halogen atom, a linear or branched C₁-C₆ alkyl group, optionally containing a silicon atom, a linear or branched C₁-C₆ alkoxy group, optionally containing a silicon atom, a linear or branched C₁-C₅ alkoxycarbonyl group, or a linear or branched C₁-C₆ alkylsulfonamide group, optionally containing a silicon atom or an amino acid function,

p′ represents an integer between 0 and 4 inclusive,

q′ represents 0 or 1,

R₅ represents hydrogen or an OH group,

R₆ represents hydrogen, a linear or branched C₁-C₆ alkyl group, optionally containing a silicon atom, a cyano group, a C₁-C₆ alkylsulfonyl group, or a phenylsulfonyl group,

R₇ represents a linear or branched C₁-C₆ alkyl group, optionally containing a silicon atom, or a phenyl group which can form a bicycle and which is optionally substituted with one or two radicals R₄,

or R₆ and R₇ together form a monocyclic, bicyclic or tricyclic C₂-C₁₀ hydrocarbon-based residue, optionally interrupted with one or more nitrogen, sulfur and oxygen atoms and possibly containing another carbonyl, and optionally substituted with a linear or branched C₁-C₈ alkylsulfonamide group optionally containing a silicon atom or an amino acid function, on the condition that, when n′=1, R₆ and R₇ do not form a camphor nucleus.

As examples of insoluble compounds of formula (X), in which n′=1, which screen out UV radiation and which have a mean particle size of between 10 nm and 5 μm, mention may be made of the following families:
compounds of the styryl ketone type as described in application JP 04 134 042, such as 1-(3,4-dimethoxyphenyl)-4,4-dimethylpent-1-en-3-one:

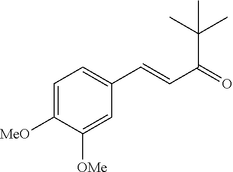 [SMILES: COc1ccc(/C=C/C(=O)C(C)(C)C)cc1OC]

compounds of the benzylidene cineole type such as those described in the article by E. Mariani et al., 16th IFSCC Congress, New York (1990), such as 1,3,3-trimethyl-5-(4-methoxybenzylidene)-2-oxabicyclo [2.2.2]octan-6-one:

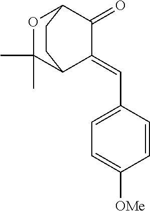 [SMILES: COc1ccc(/C=C2/C(=O)C3CCC2C(C)(C)O3)cc1]

compounds of the benzylidene chromanone type such as those described in application JP 04 134 043, for instance 3-(4-methoxybenzylidene)-2,3,4a,8a-tetrahydrochromen-4-one:

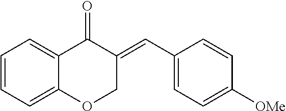 [SMILES: COc1ccc(/C=C2\COc3ccccc3C2=O)cc1]

compounds of the benzylidene thiochromanone type such as those described in application JP 04 134 043, for instance 3-(4-methoxybenzylidene)-2,3,4a,8a-tetrahydrochromen-4-thione:

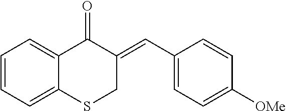 [SMILES: COc1ccc(/C=C2\CSc3ccccc3C2=O)cc1]

compounds of the benzylidene quinuclidinone type such as those described in application EP 0 576 974, for instance 4-methoxybenzylidene-1-azabicyclo[2.2.2]octan-3-one:

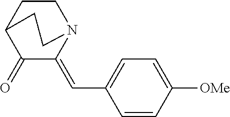 [SMILES: COc1ccc(/C=C2/C(=O)C3CCN2CC3)cc1]

compounds of the benzylidene cycloalcanone type such as those described in application FR 2 395 023, for instance 2-(4-methoxybenzylidene)cyclopentanone and 2-(4-methoxybenzylidene)cyclohexanone:

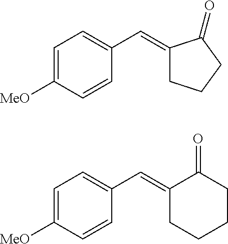 [SMILES: COc1ccc(/C=C2\CCCC2=O)cc1 ; COc1ccc(/C=C2\CCCCC2=O)cc1]

compounds of the benzylidene hydantoin type such as those described in application JP 01 158 090, for instance 5-(3,4-dimethoxybenzylidene)imidazolidine-2,4-dione:

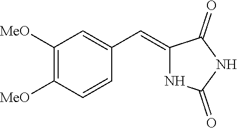 [SMILES: COc1ccc(/C=C2\NC(=O)NC2=O)cc1OC]

compounds of the benzylidene indanone type such as those described in application JP 04 134 043, for instance 2-(4-methoxybenzylidene)indan-1-one:

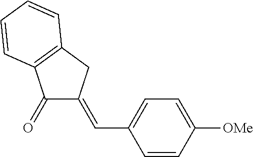 [SMILES: COc1ccc(/C=C2\Cc3ccccc3C2=O)cc1]

compounds of the benzylidene tetralone type such as those described in application JP 04 134 043, for instance 2-(4-methoxybenzylidene)-3,4-dihydro-2H-naphthalen-1-one:

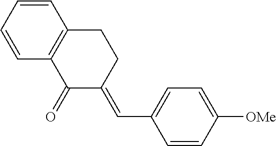 [SMILES: COc1ccc(/C=C2\CCc3ccccc3C2=O)cc1]

compounds of the benzylidene furanone type such as those described in application EP 0 390 683, for instance 4-(4-methoxybenzylidene)-2,2,5,5-tetramethyldihydrofuran-3-one:

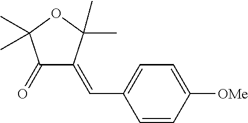 [SMILES: COc1ccc(/C=C2/C(=O)C(C)(C)OC2(C)C)cc1]

compounds of the benzylidene benzofuranone type such as those described in application JP 04 134 041, for instance 2-benzylidenebenzofuran-3-one:

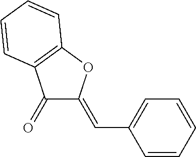 [SMILES: O=C1/C(=C/c2ccccc2)Oc2ccccc21]

compounds of the benzylidene indanedione type such as 2-(3,5-di(tert-butyl)-4-hydroxybenzylidene)indan-1,3-dione:

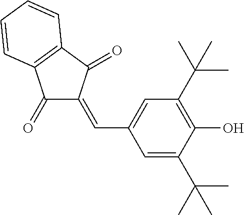 [SMILES: CC(C)(C)c1cc(C=C2C(=O)c3ccccc3C2=O)cc(C(C)(C)C)c1O]

compounds of the benzylidene benzothiofuranone type such as those described in application JP 04,134,043), for instance 2-benzylidenebenzo[b]thiophen-3-one:

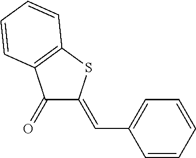 [SMILES: O=C1/C(=C/c2ccccc2)Sc2ccccc21]

compounds of the benzylidene barbiturate type such as 5-(4-methoxybenzylidene)-1,3-dimethylpyrimidine-2,4,6-trione:

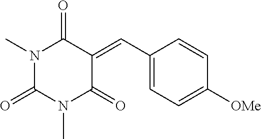 [SMILES: COc1ccc(C=C2C(=O)N(C)C(=O)N(C)C2=O)cc1]

compounds of the benzylidene pyrazolone type such as 4-(4-methoxybenzylidene)-5-methyl-2-phenyl-2,4-dihydropyrazol-3-one:

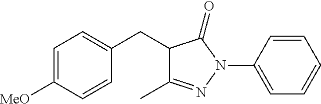 [SMILES: COc1ccc(CC2C(=O)N(c3ccccc3)N=C2C)cc1]

compounds of the benzylidene imidazolone type such as 5-(4-methoxybenzylidene)-2-phenyl-3,5-dihydroimidazol-4-one:

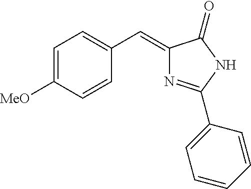 [SMILES: COc1ccc(/C=C2\N=C(c3ccccc3)NC2=O)cc1]

compounds of the chalcone type such as 1-(2-hydroxy-4-methoxyphenyl)-3-phenylpropenone:

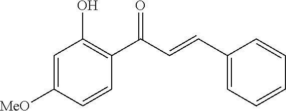 [SMILES: COc1ccc(C(=O)/C=C/c2ccccc2)c(O)c1]

benzylidene one compounds as described in document FR 2 506 156, for instance 3-hydroxy-1-(2-hydroxy-4-methoxyphenyl)-3-phenylpropenone:

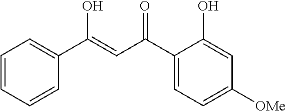 [SMILES: COc1ccc(C(=O)/C=C(\O)c2ccccc2)c(O)c1]

As examples of insoluble compounds of formula (X), in which n′=2, which screen out UV radiation and which have a mean particle size of between 10 nm and 5 μm, mention may be made of the following families:
compounds of the phenylene bis methylidene-nor-camphor type as described in document EP 0 693 471, for instance 1,4-phenylene-bis-{3-methylidenebicyclo[2.2.1]heptan-2-one}:

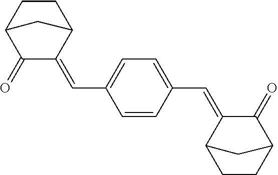 [SMILES: O=C1/C(=C/c2ccc(/C=C3/C(=O)C4CCC3C4)cc2)C2CCC1C2]

compounds of the phenylene bis methylidene camphor type as described in document FR 2 528 420, for instance 1,4-phenylene-bis-{3-methylidene-1,7,7-trimethylbicyclo[2.2.1]heptan-2-one}:

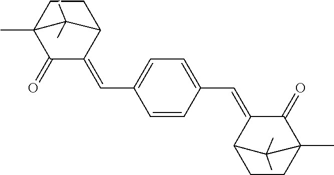

or 1,3-phenylene-bis-{3-methylidene-1,7,7-trimethylbicyclo [2.2.1]heptan-2-one}:

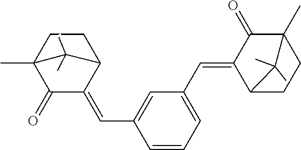

compounds of the phenylene bis methylidene camphor sulfonamide type such as those described in document FR2 529 887, for instance ethyl or 2-ethylhexyl 1,4-phenylene-bis-3,3′-methylidenecamphor-10,10′-sulfonamide:

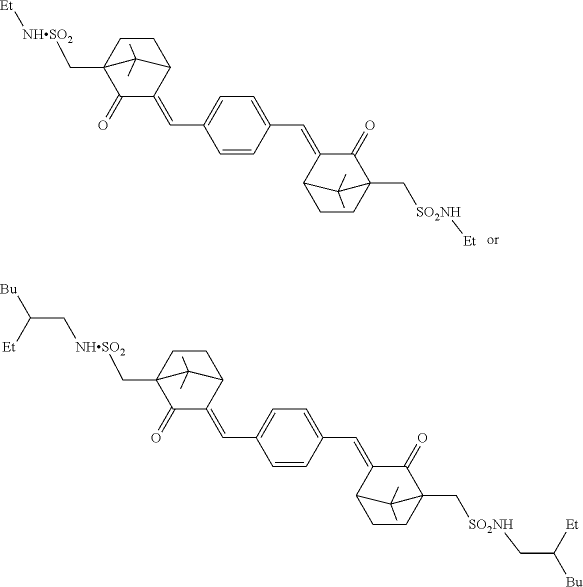

compounds of the phenylene bis methylidene cineole type as described in the article E. Mariani et al., 16th IFSCC Congress, New York (1990), for instance 1,4-phenylene-bis-{5-methylidene-3,3-dimethyl-2-oxabicyclo[2.2.2] octan-6-one}:

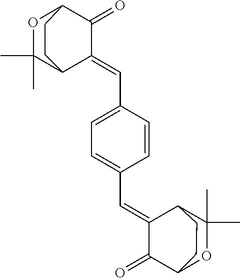

compounds of the phenylene bis methylidene ketotricyclodecane type as described in application EP 0 694 521, for instance 1,4-phenylene-bis-(octahydro-4,7-methano-6-inden-5-one):

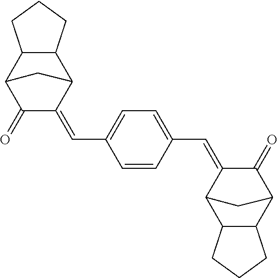 [SMILES: O=C1/C(=C/c2ccc(/C=C3/C(=O)C4CC3C3CCCC43)cc2)C2CC1C1CCCC21]

compounds of the phenylene bis alkylene ketone type such as those described in application JP 04 134 041, for instance 1,4-phenylene-bis-(4,4-dimethyl-pent-1-en-3-one):

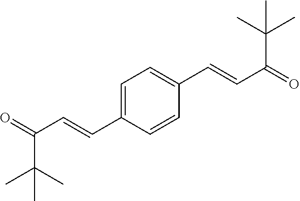 [SMILES: CC(C)(C)C(=O)/C=C/c1ccc(/C=C/C(=O)C(C)(C)C)cc1]

compounds of the phenylene bis methylidene furanone type as described in application FR 2 638 354, for instance 1,4-phenylene-bis-(4-methylidene-2,2,5,5-tetramethyldihydrofuran-3-one):

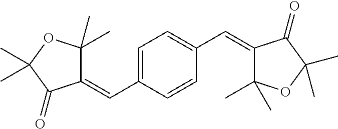

compounds of the phenylene bis methylidene quinuclidinone type such as those described in application EP 0 714 880, for instance 1,4-phenylene-bis-{2-methylidene-1-azabicyclo [2.2.2]octan-3-one}:

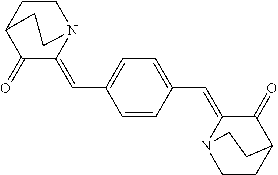 [SMILES: O=C1/C(=C/c2ccc(/C=C3/C(=O)C4CCN3CC4)cc2)N2CCC1CC2]

As compounds of formula (XI), mention may be made of the following families:
compounds of the bis benzylidene cycloalcanone type such as 2,5-dibenzylidenecyclopentanone:

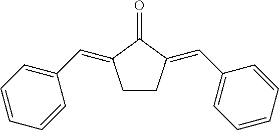 [SMILES: O=C1/C(=C/c2ccccc2)CC/C1=C\c1ccccc1]

compounds of the gamma pyrone type as described in document JP 04 290 882, for instance 2,6-bis-(3,4-dimethoxyphenyl)pyran-4-one:

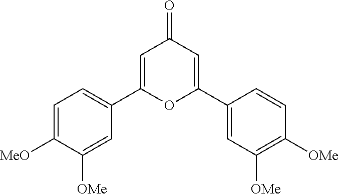 [SMILES: COc1ccc(-c2cc(=O)cc(-c3ccc(OC)c(OC)c3)o2)cc1OC]

Among these insoluble organic compounds which screen out UV radiation, of the aryl vinylene ketone type, preference is given most particularly to the compounds of formula (X) in which n′=2.

H/ Phenylene bis-benzoxazinones

Among the insoluble organic screening agents of the phenylene bis-benzoxazinone type, mention may be made of those corresponding to formula (XII) below:

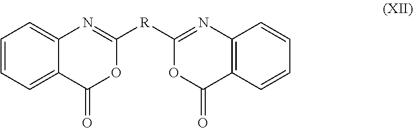

with R representing a divalent aromatic residue chosen from the formulae (e) to (h) below:

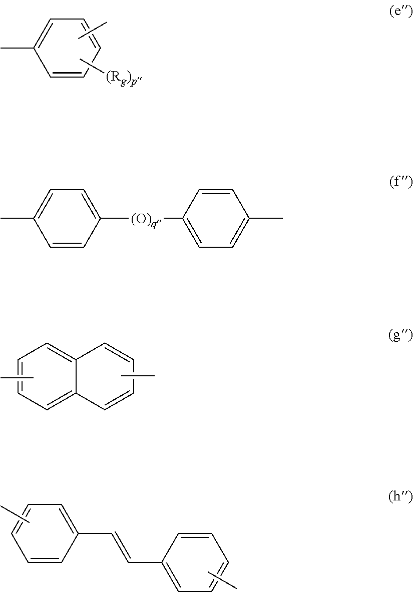

in which:

each of the symbols R₉ independently represents an OH group, a halogen atom, a linear or branched C₁-C₆ alkyl group, optionally containing a silicon atom, a linear or branched C₁-C₆ alkoxy group, optionally containing a silicon atom, a linear or branched C₁-C₅ alkoxycarbonyl group, or a linear or branched C₁-C₆ alkylsulfonamide group, optionally containing a silicon atom or an amino acid function,

p″ represents an integer between 0 and 4 inclusive,

q″ represents 0 or 1,

As examples of insoluble compounds of formula (XII), which screen out UV radiation and which have a mean particle size of between 10 nm and 5 μm, mention may be made of the following derivatives:
2,2′-p-phenylenebis(3,1-benzoxazin-4-one), sold in particular under the trade name Cyasorb UV-3638® by the company Cytec,
2,2′-(4,4′-biphenylene)bis(3,1-benzoxazin-4-one),
2,2′-(2,6-naphthylene)bis(3,1-benzoxazin-4-one).

I/ Acrylonitrile Amide, Sulfonamide or Carbamate Derivatives

Among the insoluble organic screening agents of the acrylonitrile amide, sulfonamide or carbamate derivative type, mention may be made of those corresponding to formula (XIII) below:

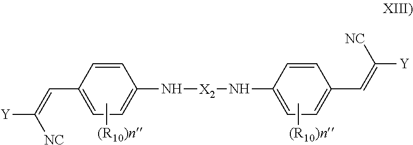

in which:

R₁₀ represents a linear or branched C₁-C₈ alkyl group,

n′″ is 0, 1 or 2,

X₂ represents a divalent radical of formula —(C═O)—R₁₁—(C═O)—, —SO₂—R₁₁—SO₂— or —(C═O)—O—R₁₁—O—(C═O)—,

Y represents a radical —(C═O)—R₁₂ or —SO₂R₁₃,

R₁₁ represents a single bond or a linear or branched C₁-C₃₀ alkylene or C₃-C₃₀ alkenylene divalent radical which may bear one or more hydroxyl substituents and which may contain, in the carbon-based chain, one or more heteroatoms chosen from oxygen, nitrogen and silicon atoms,

R₁₂ represents a radical —OR₁₄ or —NHR₁₄,

R₁₃ represents a linear or branched C₁-C₃₀ alkyl radical, or a phenyl nucleus which is unsubstituted or substituted with C₁-C₄ alkyl or alkoxy radicals,

R₁₄ represents a linear or branched C₁-C₃₀ alkyl or C₃-C₃₀ alkenyl radical which may bear one or more hydroxyl substituents and which may contain, in the carbon-based chain, one or more heteroatoms chosen from oxygen, nitrogen and silicon atoms.

Although, in formula (XIII) above, only the isomers in which the cyano substituent is in the cis position relative to the para-aminophenyl substituent are represented, this formula should be understood as also encompassing the corresponding trans isomers; for each of the two double bonds and independently, the cyano and para-aminophenyl substituents may be in the cis or trans configuration relative to each other.

By way of example, mention may be made of the dimer of 2-ethylhexyl 2-cyano-3-[4-(acetylamino)phenyl]acrylate of formula:

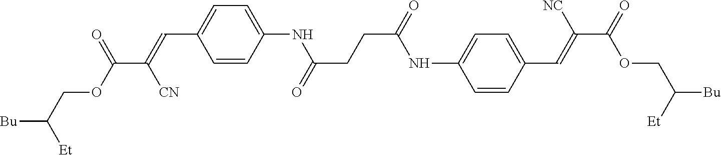

J/ Polyvalent Metals

Another particular family of insoluble organic screening agents in accordance with the invention are the salts of polyvalent metals (for example Ca²⁺, Zn²⁺, Mg²⁺, Ba²⁺, Al³⁺ or Zr⁴⁺) of sulfonic or carboxylic organic screening agents such as the polyvalent metal salts of sulfonated derivatives of benzylidenecamphor, such as those described in application FR-A 2 639 347; the polyvalent metal salts of sulfonated derivatives of benzimidazole, such as those described in application EP-A-893119; the polyvalent metal salts of cinnamic acid derivatives, such as those described in patent application JP-87 166 517.

Mention may also be made of the metal, ammonium or substituted-ammonium complexes of UVA and/or UVB organic screening agents as described in patent applications WO93/10753, WO93/11095 and WO95/05150.

Among the insoluble organic UV-screening agents, mention may also be made of the compound 1,1′-(1,4-piperazinediyl)bis[1-[2-[4-(diethylamino)-2-hydroxybenzoyl]phenyl]methanone (CAS 919803-06-8) having the following structure:

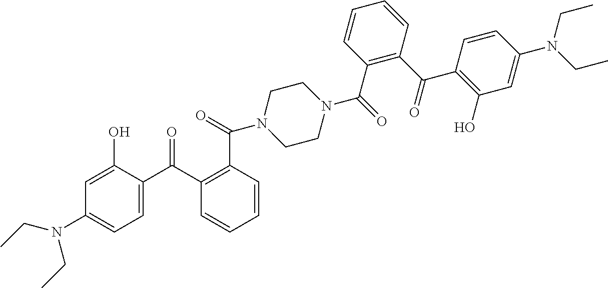 [SMILES: CCN(CC)c1ccc(C(=O)c2ccccc2C(=O)N2CCN(C(=O)c3ccccc3C(=O)c3ccc(N(CC)CC)cc3O)CC2)c(O)c1]

as described in patent application WO 2007/071 584; this compound advantageously being used in micronized form (mean size of 0.02 to 2 μm), which may be obtained, for example, according to the micronization process described in patent applications GB-A-2 303 549 and EP-A-893 119, and in particular in the form of an aqueous dispersion.

According to a particularly preferred form of the invention, use will be made of the insoluble organic UV-screening agents chosen from:

(i) symmetrical triazine screening agents substituted with naphthalenyl groups or polyphenyl groups described in patent U.S. Pat. No. 6,225,467, patent application WO 2004/085 412 (see compounds 6 and 9) or the document “Symmetrical Triazine Derivatives”, IP.COM IPCOM000031257 Journal, INC West Henrietta, N.Y., US (20 Sep. 2004), in particular 2,4,6-tris(diphenyl)triazine and 2,4,6-tris(terphenyl)triazine, which is also mentioned in patent applications WO 06/035 000, WO 06/034 982, WO 06/034 991, WO 06/035 007, WO 2006/034 992 and WO 2006/034 985, these compounds advantageously being used in micronized form (mean particle size of 0.02 to 3 μm), which may be obtained, for example, according to the micronization process described in patent applications GB-A-2 303 549 and EP-A-893 119, and in particular in aqueous dispersion form;

(ii) the methylenebis(hydroxyphenylbenzotriazole) compounds of formula (IV) below:

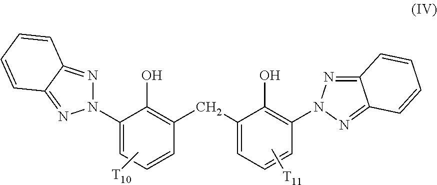

in which the radicals T₁₀ and T₁₁, which may be identical or different, denote a C₁-C₁₈ alkyl radical which may be substituted with one or more radicals chosen from C₁-C₄ alkyl, C₅-C₁₂ cycloalkyl or an aryl residue;

(iii) and mixtures thereof.

According to a particularly preferred form of the invention, the methylenebis(hydroxyphenylbenzotriazole) compounds of formula (IV) are in the form of an aqueous dispersion of particles having a mean particle size which ranges from 0.01 to 5 μm and more preferentially from 0.01 to 2 μm and more particularly from 0.020 to 2 μm with at least one surfactant of structure CₙH₂ₙ₊₁ O(C₆H₁₀O₅)ₓH in which n is an integer from 8 to 16 and x is the mean degree of polymerization of the unit (C₆H₁₀O₅) and ranges from 1.4 to 1.6 as defined previously. Said surfactant is preferably used at a concentration ranging from 1% to 50% by weight, and more preferentially from 5% to 40% by weight, relative to the benzotriazole screening agent, and the amount of benzotriazole screening agent of formula (I) in the aqueous dispersion preferably ranges from 10% to 50% by weight, and more preferentially from 30% to 50% by weight, relative to the total weight of the dispersion.

The mean particle diameter is measured using a particle size distribution analyzer of the Culter N4 PLUS® type manufactured by Beckman Coulter Inc.

According to a particularly preferred form of the invention, the methylenebis(hydroxyphenylbenzotriazole) compounds of formula (IV) may be in the form of an aqueous dispersion of particles having a mean particle size which ranges from 0.02 to 2 μm and more preferentially from 0.01 to 1.5 μm and more particularly from 0.02 to 1 μm in the presence of at least one polyglycerol mono(C₈-C₂₀)alkyl ester having a degree of glycerol polymerization of at least 5, such as the aqueous dispersions described in patent application WO2009/063392.

As an example of surfactants which are polyglycerol mono(C₈-C₂₀)alkyl esters, mention may be made of decaglyceryl caprate, decaglyceryl laurate, decaglyceryl myristate, decaglyceryl oleate, decaglyceryl stearate, decaglyceryl isostearate, hexaglyceryl caprate, hexaglyceryl laurate, hexaglyceryl myristate, hexaglyceryl oleate, hexaglyceryl stearate, hexaglyceryl isostearate, pentaglyceryl caprate, pentaglyceryl laurate, pentaglyceryl myristate, pentaglyceryl oleate, pentaglyceryl stearate, and pentaglyceryl isostearate.

Use will more particularly be made of:
decaglyceryl caprate such as the products sold under the following trade names: Sunsoft Q10Y®, Sunsoft Q10S®, Sunsoft Q12Y®, Sunsoft Q125®, Sunsoft M12J® by the company Taiyo Kagaku Co. Ltd., Nikkol Decaglyn 1-L by the company Nikko Chemicals Co. Ltd, Ryoto-Polyglycerylester L-10D® and L-7D® by the company Mitsubishi-Kagaku Co. Ltd.,
decaglyceryl laurate such as the products sold under the following trade names: Sunsoft Q14Y®, Sunsoft Q145®, Sunsoft Q12Y®, Sunsoft Q125®, Sunsoft M12J® by the company Taiyo Kagaku Co. Ltd., Nikkol Decaglyn 1-M® by the company Nikko Chemicals Co. Ltd, Ryoto-Polyglycerylester M-10D® and M-7D® by the company Mitsubishi-Kagaku Co. Ltd.,
decaglyceryl stearate such as the products sold under the following trade names: Sunsoft Q18Y®, Sunsoft Q185®, Sunsoft Q12Y®, Sunsoft Q125®, Sunsoft M12J® by the company Taiyo Kagaku Co. Ltd., Nikkol Decaglyn 1-SV by the company Nikko Chemicals Co. Ltd, Ryoto-Polyglycerylester S-15D® by the company Mitsubishi-Kagaku Co. Ltd.,
hexaglyceryl caprate such as the products sold under the following trade names: Nikkol Hexaglyn 1-L® by the company Nikko Chemicals Co. Ltd, Glysurf 6ML by the company Aoki Oil Industrial Co. Ltd., Unigly GL-106® by the company Nippon Oil & Fats Co. Ltd.,
hexaglyceryl myristate such as the products sold under the following trade names: Nikkol Hexaglyn 1-M®, Nikkol Hexaglyn 1-OV® by the company Nikko Chemicals Co. Ltd, Glysurf 6ML® by the company Aoki Oil Industrial Co. Ltd., Unigly GL-106 by the company Nippon Oil & Fats Co. Ltd.,
hexaglyceryl stearate such as the products sold under the following trade names: Nikkol Hexaglyn 1-M®, Nikkol Hexaglyn 1-SV® by the company Nikko Chemicals Co. Ltd, EmalexMSG-6K® by the company Nihon-Emulsion Co. Ltd., Unigly GL-106 by the company Nippon Oil & Fats Co. Ltd.,
hexaglyceryl isostearate such as the products sold under the following trade names: Matsumate MI-610® by the company Matsumoto Fine Chemical Co. Ltd,
pentaglyceryl caprate such as the products sold under the following trade names: Sunsoft A10E®, by the company Taiyo Kagaku Co. Ltd.,
pentaglyceryl laurate such as the products sold under the following trade names: Sunsoft A12E® Sunsoft A121E®, by the company Taiyo Kagaku Co. Ltd.,
pentaglyceryl myristate such as the products sold under the following trade names: Sunsoft A14E®, Sunsoft A141E®, by the company Taiyo Kagaku Co. Ltd.,
pentaglyceryl oleate such as the products sold under the following trade names: Sunsoft A17E®, Sunsoft A171E®, by the company Taiyo Kagaku Co. Ltd.,
pentaglyceryl stearate such as the products sold under the following trade names: Sunsoft A18E®, Sunsoft A181E®, by the company Taiyo Kagaku Co. Ltd.

Among these surfactants, those having an HLB greater than or equal to 14.5, and more preferentially greater than or equal to 15, are preferably used. As examples of surfactants which are mono-(C₈-C₂₀)alkyl esters of polyglycerol having a degree of glycerol polymerization of at least 5 and having an HLB greater than or equal to 14.5, mention may be made of decaglyceryl caprate, decaglyceryl laurate, decaglyceryl myristate, decaglyceryl oleate, decaglyceryl stearate, decaglyceryl isostearate, hexaglyceryl laurate, pentaglyceryl caprate, pentaglyceryl laurate, pentaglyceryl myristate, pentaglyceryl oleate, and pentaglyceryl stearate. As examples of surfactants which are mono-(C₈-C₂₀)alkyl esters of polyglycerol having a degree of glycerol polymerization of at least 5 and having an HLB greater than or equal to 15, mention may be made of decaglyceryl caprate and decaglyceryl laurate.

The amount of methylenebis(hydroxyphenylbenzotriazole) compound of formula (IV) in the aqueous dispersion preferably ranges from 10% to 50% by weight, and more preferentially from 30% to 50% by weight, relative to the total weight of the dispersion.

Preferentially, the methylenebis(hydroxyphenylbenzotriazole) compound/mono-(C₈-C₂₀)alkyl ester of polyglycerol weight ratio ranges from 0.05 to 0.5, and more preferentially from 0.1 to 0.3.

In these aqueous dispersions, use will preferentially be made, as methylenebis(hydroxyphenylbenzotriazole) compound of formula (IV), of the compound 2,2′-methylenebis[6-(2H-benzotriazol-2-yl)-4-(1,1,3,3-tetramethylbutyl)phenol] having the structure:

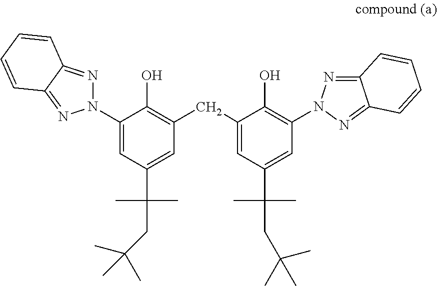 [SMILES: CC(C)(C)CC(C)(C)c1cc(Cc2cc(C(C)(C)CC(C)(C)C)cc(-n3nc4ccccc4n3)c2O)c(O)c(-n2nc3ccccc3n2)c1]

such as the commercial product sold under the name Tinosorb M® by BASF which is an aqueous dispersion comprising decylglucoside, xanthan gum and propylene glycol (INCI name: Methylene Bis-Benzotriazolyl Tetramethylbutylphenol (and) Aqua (and) Decyl Glucoside (and) Propylene Glycol (and) Xanthan Gum).

The insoluble organic UV screening agent(s) are present at an active material concentration preferably ranging from 0.1% to 30% by weight approximately and more particularly from 0.5% to 20% by weight relative to the total weight of the composition.

B/ Insoluble Inorganic UV-Screening Agents

The inorganic UV-screening agents used in accordance with the present invention are metal oxide pigments. More preferentially, the inorganic UV-screening agents of the invention are metal oxide particles with a mean elementary particle size of less than or equal to 0.50 μm, more preferentially between 0.005 and 0.50 μm, even more preferentially between 0.01 and 0.2 μm, better still between 0.01 and 0.1 μm and more particularly preferentially between 0.015 and 0.05 μm.

The term “elementary size” means the size of non-aggregated particles.

They may be chosen in particular from titanium oxide, zinc oxide, iron oxide, zirconium oxide and cerium oxide, or mixtures thereof.

Such coated or uncoated metal oxide pigments are described in particular in patent application EP-A-0 518 773. Commercial pigments that may be mentioned include in particular the products sold by the companies Sachtleben Pigments, Tayca, Merck and Degussa.

The metal oxide pigments may be coated or uncoated.

The coated pigments are pigments that have undergone one or more surface treatments of chemical, electronic, mechanochemical and/or mechanical nature with compounds such as amino acids, beeswax, fatty acids, fatty alcohols, anionic surfactants, lecithins, sodium, potassium, zinc, iron or aluminum salts of fatty acids, metal alkoxides (of titanium or aluminum), polyethylene, silicones, proteins (collagen, elastin), alkanolamines, silicon oxides, metal oxides or sodium hexametaphosphate.

The coated pigments are more particularly titanium oxides that have been coated:
with silica, such as the product Sunveil from the company Ikeda,
with silica and iron oxide, such as the product Sunveil F from the company Ikeda,
with silica and alumina, such as the products Microtitanium Dioxide MT 500 SA and Microtitanium Dioxide MT 100 SA from the company Tayca and Tioveil from the company Tioxide,
with alumina, such as the products Tipaque TTO-55 (B) and Tipaque TTO-55 (A) from the company Ishihara, and UVT 14/4 from the company Sachtleben Pigments,
with alumina and aluminum stearate, such as the products Microtitanium Dioxide MT 100 T, MT 100 TX, MT 100 Z and MT-01 from the company Tayca, the products Solaveil CT-10 W and Solaveil CT 100 from the company Uniqema and the product Eusolex T-AVO from the company Merck,
with silica, alumina and alginic acid, such as the product MT-100 AQ from the company Tayca,
with alumina and aluminum laurate, such as the product Microtitanium Dioxide MT 100 S from the company Tayca,
with iron oxide and iron stearate, such as the product Microtitanium Dioxide MT 100 F from the company Tayca,
with zinc oxide and zinc stearate, such as the product BR 351 from the company Tayca,
with silica and alumina and treated with a silicone, such as the products Microtitanium Dioxide MT 600 SAS, Microtitanium Dioxide MT 500 SAS or Microtitanium Dioxide MT 100 SAS from the company Tayca,
with silica, alumina and aluminum stearate and treated with a silicone, such as the product STT-30-DS from the company Titan Kogyo,
with silica and treated with a silicone, such as the product UV-Titan X 195 from the company Sachtleben Pigments,
with alumina and treated with a silicone, such as the products Tipaque TTO-55 (S) from the company Ishihara or UV Titan M 262 from the company Sachtleben Pigments,
with triethanolamine, such as the product STT-65-S from the company Titan Kogyo,
with stearic acid, such as the product Tipaque TTO-55 (C) from the company Ishihara,
with sodium hexametaphosphate, such as the product Microtitanium Dioxide MT 150 W from the company Tayca,
TiO₂ treated with octyltrimethylsilane, sold in particular under the trade name T 805 by the company Degussa Silices,
TiO₂ treated with a polydimethylsiloxane, sold in particular under the trade name 70250 Cardre UF TiO2SI3 by the company Cardre,
anatase/rutile TiO₂ treated with a polydimethylhydrogenosiloxane, sold in particular under the trade name Microtitanium Dioxide USP Grade Hydrophobic by the company Color Techniques.

Mention may also be made of TiO₂ pigments doped with at least one transition metal such as iron, zinc or manganese and more particularly manganese. Preferably, said doped pigments are in the form of an oily dispersion. The oil present in the oily dispersion is preferably chosen from triglycerides including those of capric/caprylic acids. The oily dispersion of titanium oxide particles may also comprise one or more dispersants, for instance a sorbitan ester, for instance sorbitan isostearate, or a polyoxyalkylenated fatty acid ester of glycerol, for instance TRI-PPG-3 myristyl ether citrate and polyglyceryl-3 polyricinoleate. Preferably, the oily dispersion of titanium oxide particles comprises at least one dispersant chosen from polyoxyalkylenated fatty acid esters of glycerol. Mention may be made more particularly of the oily dispersion of TiO₂ particles doped with manganese in capric/caprylic acid triglyceride in the presence of TRI-PPG-3 myristyl ether citrate and polyglyceryl-3 polyricinoleate and sorbitan isostearate having the INCI name: titanium dioxide (and) TRI-PPG-3 myristyl ether citrate (and) polyglyceryl-3 ricinoleate (and) sorbitan isostearate, for instance the product sold especially under the trade name Optisol TD50 by the company Croda.

The uncoated titanium oxide pigments are sold, for example, by the company Tayca under the trade names Microtitanium Dioxide MT 500 B or Microtitanium Dioxide MT 600 B, by the company Degussa under the name P 25, by the company Wackher under the name Transparent titanium oxide PW, by the company Miyoshi Kasei under the name UFTR, by the company Tomen under the name ITS and by the company Tioxide under the name Tioveil AQ.

The uncoated zinc oxide pigments are for example:
those sold especially under the name Z-Cote by the company Sunsmart;
those sold especially under the name Nanox by the company Elementis;
those sold especially under the name Nanogard WCD 2025 by the company Nanophase Technologies.

The coated zinc oxide pigments are for example:
those sold especially under the name Zinc Oxide CS-5 by the company Toshibi (ZnO coated with polymethylhydrosiloxane);
those sold especially under the name Nanogard Zinc Oxide FN by the company Nanophase Technologies (as a 40% dispersion in Finsolv TN, C₁₂-C₁₅ alkyl benzoate);
those sold especially under the names Daitopersion Zn-30 and Daitopersion Zn-50 by the company Daito (dispersions in cyclopolymethylsiloxane/oxyethylenated polydimethylsiloxane, containing 30% or 50% of zinc oxides coated with silica and polymethylhydrosiloxane);
those sold especially under the name NFD Ultrafine ZnO by the company Daikin (ZnO coated with perfluoroalkyl phosphate and copolymer based on perfluoroalkylethyl as a dispersion in cyclopentasiloxane);
those sold especially under the name SPD-Z1 by the company Shin-Etsu (ZnO coated with silicone-grafted acrylic polymer, dispersed in cyclodimethylsiloxane);
those sold especially under the name Escalol Z100 by the company ISP (alumina-treated ZnO dispersed in an ethylhexyl methoxycinnamate/PVP-hexadecene copolymer/methicone mixture);
those sold especially under the name Fuji ZnO-SMS-10 by the company Fuji Pigment (ZnO coated with silica and polymethylsilsesquioxane);
those sold especially under the name Nanox Gel TN by the company Elementis (ZnO dispersed at a concentration of 55% in C₁₂-C₁₅ alkyl benzoate with hydroxystearic acid polycondensate).

The uncoated cerium oxide pigments may, for example, be those sold under the name Colloidal Cerium Oxide by the company Rhône-Poulenc.

The uncoated iron oxide pigments are sold, for example, by the company Arnaud under the names Nanogard WCD 2002 (FE 45B), Nanogard Iron FE 45 BL AQ, Nanogard FE 45R AQ and Nanogard WCD 2006 (FE 45R) or by the company Mitsubishi under the name TY-220.

The coated iron oxide pigments are sold, for example, by the company Arnaud under the names Nanogard WCD 2008 (FE 45B FN), Nanogard WCD 2009 (FE 45B 556), Nanogard FE 45 BL 345 and Nanogard FE 45 BL or by the company BASF under the name Transparent Iron Oxide.

Mention may also be made of mixtures of metal oxides, in particular of titanium dioxide and of cerium dioxide, including the equal-weight mixture of titanium dioxide and cerium dioxide coated with silica, sold by the company Ikeda under the name Sunveil A, and also the mixture of titanium dioxide and zinc dioxide coated with alumina, silica and silicone, such as the product M 261 sold by the company Sachtleben Pigments, or coated with alumina, silica and glycerol, such as the product M 211 sold by the company Sachtleben Pigments.

According to the invention, coated or uncoated titanium oxide pigments are particularly preferred.

The inorganic insoluble UV-screening agents of the invention are preferably present in the compositions according to the invention in a content ranging from 0.1% to 50% by weight, more particularly from 0.1% to 40% by weight and in particular from 0.5% to 30% by weight relative to the total weight of the composition.

As outlined previously, according to a particularly preferred embodiment, the UV-screening agent(s) are chosen from water-soluble organic UV-screening agents, liposoluble organic UV-screening agents, and mixtures thereof.

According to a particular form of the invention, the composition comprises:
at least one aqueous phase gelled with at least one non-starchy hydrophilic gelling agent chosen from crosslinked and/or neutralized 2-acrylamido-2-methylpropanesulfonic acid (AMPS®) copolymers, and more particularly a copolymer of AMPS® and of hydroxyethyl acrylate,
at least one oily phase gelled with at least one lipophilic gelling agent chosen from hectorites modified with a C10 to C22 ammonium chloride, and more particularly a hectorite modified with distearyldimethylammonium chloride.

Said phases forming therein a macroscopically homogeneous mixture;

said composition also comprising at least one UV-screening agent chosen from water-soluble organic UV-screening agents, liposoluble organic UV-screening agents, and mixtures thereof, and preferably liposoluble organic UV-screening agents.

Aqueous Phase

The aqueous phase of a composition according to the invention comprises water and optionally a water-soluble solvent.

In the present invention, the term “water-soluble solvent” denotes a compound that is liquid at room temperature and water-miscible (miscibility with water of greater than 50% by weight at 25° C. and atmospheric pressure).

The water-soluble solvents that may be used in the composition of the invention may also be volatile.

Among the water-soluble solvents that may be used in the composition in accordance with the invention, mention may be made especially of lower monoalcohols containing from 1 to 5 carbon atoms such as ethanol and isopropanol, glycols containing from 2 to 8 carbon atoms such as ethylene glycol, propylene glycol, 1,3-butylene glycol and dipropylene glycol, C₃ and C₄ ketones and C₂-C₄ aldehydes.

The aqueous phase (water and optionally the water-miscible solvent) may be present in the composition in a content ranging from 5% to 95%, better still from 30% to 80% by weight and preferably from 40% to 75% by weight relative to the total weight of said composition.

According to another embodiment variant, the aqueous phase of a composition according to the invention may comprise at least one C₂-C₃₂ polyol.

For the purposes of the present invention, the term “polyol” should be understood as meaning any organic molecule comprising at least two free hydroxyl groups.

Preferably, a polyol in accordance with the present invention is present in liquid form at room temperature.

A polyol that is suitable for use in the invention may be a compound of linear, branched or cyclic, saturated or unsaturated alkyl type, bearing on the alkyl chain at least two —OH functions, in particular at least three —OH functions and more particularly at least four —OH functions.

The polyols that are advantageously suitable for formulating a composition according to the present invention are those especially containing from 2 to 32 carbon atoms and preferably 3 to 16 carbon atoms.

Advantageously, the polyol may be chosen, for example, from ethylene glycol, pentaerythritol, trimethylolpropane, propylene glycol, 1,3-propanediol, butylene glycol, isoprene glycol, pentylene glycol, hexylene glycol, glycerol, polyglycerols such as glycerol oligomers, for instance diglycerol, and polyethylene glycols, and mixtures thereof.

According to a preferred embodiment of the invention, said polyol is chosen from ethylene glycol, pentaerythritol, trimethylolpropane, propylene glycol, glycerol, polyglycerols, polyethylene glycols and mixtures thereof.

According to a particular mode, the composition of the invention may comprise at least propylene glycol.

According to another particular mode, the composition of the invention may comprise at least glycerol.

Oily Phase

For the purposes of the invention, an oily phase comprises at least one oil.

The term “oil” means any fatty substance that is in liquid form at room temperature and atmospheric pressure.

An oily phase that is suitable for preparing the cosmetic compositions according to the invention may comprise hydrocarbon-based oils, silicone oils, fluoro oils or non-fluoro oils, or mixtures thereof.

The oils may be volatile or non-volatile.

They may be of animal, plant, mineral or synthetic origin. According to one embodiment variant, oils of silicone origin are preferred.

For the purposes of the present invention, the term “nonvolatile oil” means an oil with a vapor pressure of less than 0.13 Pa.

For the purposes of the present invention, the term “silicone oil” means an oil comprising at least one silicon atom, and in particular at least one Si—O group.

The term “fluoro oil” means an oil comprising at least one fluorine atom.

The term “hydrocarbon-based oil” means an oil mainly containing hydrogen and carbon atoms.

The oils may optionally comprise oxygen, nitrogen, sulfur and/or phosphorus atoms, for example in the form of hydroxyl or acid radicals.

For the purposes of the invention, the term “volatile oil” means any oil that is capable of evaporating on contact with the skin in less than one hour, at room temperature and atmospheric pressure. The volatile oil is a volatile cosmetic compound, which is liquid at room temperature, especially having a nonzero vapor pressure, at room temperature and atmospheric pressure, especially having a vapor pressure ranging from 0.13 Pa to 40 000 Pa (10⁻³ to 300 mmHg), in particular ranging from 1.3 Pa to 13 000 Pa (0.01 to 100 mmHg) and more particularly ranging from 1.3 Pa to 1300 Pa (0.01 to 10 mmHg).

Volatile Oils

The volatile oils may be hydrocarbon-based oils or silicone oils.

Among the volatile hydrocarbon-based oils containing from 8 to 16 carbon atoms, mention may be made especially of branched C₈-C₁₆ alkanes, for instance C₈-C₁₆ isoalkanes (also known as isoparaffins), isododecane, isodecane, isohexadecane and, for example, the oils sold under the trade names Isopar or Permethyl, branched C₈-C₁₆ esters, for instance isohexyl neopentanoate, and mixtures thereof. Preferably, the volatile hydrocarbon-based oil is selected from volatile hydrocarbon-based oils containing from 8 to 16 carbon atoms, and mixtures thereof, in particular from isododecane, isodecane and isohexadecane, and is especially isohexadecane.

Mention may also be made of volatile linear alkanes comprising from 8 to 16 carbon atoms, in particular from 10 to 15 carbon atoms and more particularly from 11 to 13 carbon atoms, for instance n-dodecane (C₁₂) and n-tetradecane (C₁₄) sold by Sasol under the respective references Parafol 12-97 and Parafol 14-97, and also mixtures thereof, the undecane-tridecane mixture, mixtures of n-undecane (C₁₁) and of n-tridecane (C₁₃) obtained in Examples 1 and 2 of patent application WO 2008/155 059 from the company Cognis, and mixtures thereof.

Volatile silicone oils that may be mentioned include linear volatile silicone oils such as hexamethyldisiloxane, octamethyltrisiloxane, decamethyltetrasiloxane, tetradecamethylhexasiloxane, hexadecamethylheptasiloxane and dodecamethylpentasiloxane.

Volatile cyclic silicone oils that may be mentioned include hexamethylcyclotrisiloxane, octamethylcyclotetrasiloxane, decamethylcyclopentasiloxane and dodecamethylcyclohexasiloxane.

Non-Volatile Oils

The non-volatile oils may, in particular, be selected from non-volatile hydrocarbon-based, fluoro and/or silicone oils.

Nonvolatile hydrocarbon-based oils that may in particular be mentioned include:
hydrocarbon-based oils of animal origin,
hydrocarbon-based oils of plant origin, synthetic ethers containing from 10 to 40 carbon atoms, such as dicaprylyl ether,
synthetic esters, such as the oils of formula R₁COOR₂, in which R₁ represents a linear or branched fatty acid residue comprising from 1 to 40 carbon atoms and R₂ represents a hydrocarbon-based chain, which is in particular branched, containing from 1 to 40 carbon atoms, on condition that R₁+R₂≧10. The esters may be chosen especially from fatty acid alcohol esters, for instance cetostearyl octanoate, isopropyl alcohol esters such as isopropyl myristate or isopropyl palmitate, ethyl palmitate, 2-ethylhexyl palmitate, isopropyl stearate, octyl stearate, hydroxylated esters, such as isostearyl lactate or octyl hydroxystearate, alcohol or polyalcohol ricinoleates, hexyl laurate, neopentanoic acid esters, such as isodecyl neopentanoate or isotridecyl neopentanoate, and isononanoic acid esters, such as isononyl isononanoate or isotridecyl isononanoate,
polyol esters and pentaerythritol esters, such as dipentaerythrityl tetrahydroxystearate/tetraisostearate,
fatty alcohols that are liquid at room temperature, with a branched and/or unsaturated carbon-based chain containing from 12 to 26 carbon atoms, for instance 2-octyldodecanol, isostearyl alcohol and oleyl alcohol,
C₁₂-C₂₂ higher fatty acids, such as oleic acid, linoleic acid, linolenic acid, and mixtures thereof,
non-phenyl silicone oils, for instance caprylyl methicone, and
phenyl silicone oils, for instance phenyl trimethicones, phenyl dimethicones, phenyltrimethylsiloxydiphenylsiloxanes, diphenyl dimethicones, diphenylmethyldiphenyltrisiloxanes and 2-phenylethyl trimethylsiloxysilicates, dimethicones or phenyl trimethicone with a viscosity of less than or equal to 100 cSt, and trimethyl-pentaphenyl-trisiloxane, and mixtures thereof; and also mixtures of these various oils.

Preferably, a composition according to the invention comprises volatile and/or non-volatile silicone oils. Such silicone oils are particularly appreciated when the lipophilic gelling agent is an organopolysiloxane elastomer.

A composition according to the invention may comprise from 1% to 95% by weight and better still from 5% to 40% by weight of oil(s) relative to the total weight of said composition.

As mentioned above, the gelled oily phase according to the invention may have a threshold stress of greater than 1.5 Pa and preferably greater than 10 Pa.

The gelled oily phase according to the invention may have a threshold stress of less than 10 000 Pa, preferably less than 5000 Pa.

This threshold stress value reflects a gel-type texture of this oily phase.

Dyestuffs

A composition according to the invention may also comprise at least one particulate or non-particulate, water-soluble or water-insoluble dyestuff, preferably in a proportion of at least 0.01% by weight relative to the total weight of the composition.

For obvious reasons, this amount is liable to vary significantly with regard to the intensity of the desired color effect and of the color intensity afforded by the dyestuffs under consideration, and its adjustment clearly falls within the competence of a person skilled in the art.

A composition according to the invention may comprise from 0.01% to 25% by weight, especially from 0.1% to 25% by weight, in particular from 1% to 20% by weight and preferably from 5% to 18% by weight of dyestuffs relative to the total weight of said composition.

As stated above, the dyestuffs that are suitable for use in the invention may be water-soluble, but may also be liposoluble.

For the purposes of the invention, the term “water-soluble dyestuff” means any natural or synthetic, generally organic compound, which is soluble in an aqueous phase or water-miscible solvents and which is capable of imparting color.

As water-soluble dyes that are suitable for use in the invention, mention may be made especially of synthetic or natural water-soluble dyes, for instance FDC Red 4, DC Red 6, DC Red 22, DC Red 28, DC Red 30, DC Red 33, DC Orange 4, DC Yellow 5, DC Yellow 6, DC Yellow 8, FDC Green 3, DC Green 5, FDC Blue 1, betanine (beetroot), carmine, copper chlorophylline, methylene blue, anthocyanins (enocianin, black carrot, hibiscus and elder), caramel and riboflavin.

The water-soluble dyes are, for example, beetroot juice and caramel.

For the purposes of the invention, the term “liposoluble dyestuff” means any natural or synthetic, generally organic compound, which is soluble in an oily phase or in solvents that are miscible with a fatty substance, and which is capable of imparting color.

As liposoluble dyes that are suitable for use in the invention, mention may be made especially of synthetic or natural liposoluble dyes, for instance DC Red 17, DC Red 21, DC Red 27, DC Green 6, DC Yellow 11, DC Violet 2, DC Orange 5, Sudan red, carotenes (β-carotene, lycopene), xanthophylls (capsanthin, capsorubin, lutein), palm oil, Sudan brown, quinoline yellow, annatto and curcumin.

The coloring particulate materials may be present in a proportion of from 0.01% to 25% by weight, especially from 0.1% to 25% by weight, in particular from 1% to 20% by weight and preferably from 5% to 18% by weight of particulate materials relative to the total weight of the composition containing them.

They may especially be pigments, nacres and/or particles with metallic glints.

The term “pigments” should be understood as meaning white or colored, mineral or organic particles that are insoluble in an aqueous solution, which are intended to color and/or opacify the composition containing them.

A composition according to the invention may comprise from 0.01% to 25% by weight, especially from 0.1% to 25% by weight, in particular from 1% to 25% by weight and preferably from 5% to 18% by weight of pigments relative to the total weight of said composition.

Preferably, when the composition according to the invention is a makeup composition, it may comprise at least 5% and preferentially at least 3% by weight of pigments, relative to the total weight of said composition.

The pigments may be white or colored, and mineral and/or organic.

As mineral pigments that may be used in the invention, mention may be made of titanium oxide, titanium dioxide, zirconium oxide, zirconium dioxide, cerium oxide or cerium dioxide and also zinc oxide, iron oxide or chromium oxide, ferric blue, manganese violet, ultramarine blue and chromium hydrate, and mixtures thereof.

It may also be a pigment having a structure that may be, for example, of sericite/brown iron oxide/titanium dioxide/silica type. Such a pigment is sold, for example, under the reference Coverleaf NS or JS by the company Chemicals and Catalysts, and has a contrast ratio in the region of 30.

They may also be pigments having a structure that may be, for example, of silica microsphere type containing iron oxide. An example of a pigment having this tructure is the product sold by the company Miyoshi under the reference PC Ball PC-LL-100 P, this pigment consisting of silica microspheres containing yellow iron oxide.

Advantageously, the pigments in accordance with the invention are iron oxides and/or titanium dioxides.

The term “nacres” should be understood as meaning iridescent or non-iridescent colored particles of any shape, in particular produced by certain molluscs in their shell or alternatively synthesized, which have a color effect via optical interference.

A composition according to the invention may comprise from 0% to 15% by weight of nacres relative to the total weight of said composition.

The nacres may be chosen from nacreous pigments such as titanium mica coated with an iron oxide, titanium mica coated with bismuth oxychloride, titanium mica coated with chromium oxide, titanium mica coated with an organic dye and also nacreous pigments based on bismuth oxychloride. They may also be mica particles at the surface of which are superposed at least two successive layers of metal oxides and/or of organic dyestuffs.

Examples of nacres that may also be mentioned include natural mica coated with titanium oxide, with iron oxide, with natural pigment or with bismuth oxychloride.

Among the nacres available on the market, mention may be made of the nacres Timica, Flamenco and Duochrome (based on mica) sold by the company Engelhard, the Timiron nacres sold by the company Merck, the Prestige mica-based nacres, sold by the company Eckart, and the Sunshine synthetic mica-based nacres, sold by the company Sun Chemical.

The nacres may more particularly have a yellow, pink, red, bronze, orangey, brown, gold and/or coppery color or glint.

Advantageously, the nacres in accordance with the invention are micas coated with titanium dioxide or with iron oxide, and also bismuth oxychloride.

For the purposes of the present invention, the term “particles with a metallic glint” means any compound whose nature, size, structure and surface finish allow it to reflect the incident light, especially in a non-iridescent manner.

The particles with a metallic glint that may be used in the invention are in particular chosen from:
particles of at least one metal and/or of at least one metal derivative;
particles comprising a monomaterial or multimaterial organic or mineral substrate, at least partially coated with at least one layer with a metallic glint comprising at least one metal and/or at least one metal derivative; and
mixtures of said particles.

Among the metals that may be present in said particles, mention may be made, for example, of Ag, Au, Cu, Al, Ni, Sn, Mg, Cr, Mo, Ti, Zr, Pt, Va, Rb, W, Zn, Ge, Te and Se, and mixtures or alloys thereof. Ag, Au, Cu, Al, Zn, Ni, Mo and Cr, and mixtures or alloys thereof (for example bronzes and brasses) are preferred metals.

The term “metal derivatives” denotes compounds derived from metals, especially oxides, fluorides, chlorides and sulfides.

Illustrations of these particles that may be mentioned include aluminum particles, such as those sold under the names Starbrite 1200 EAC® by the company Siberline and Metalure® by the company Eckart and glass particles coated with a metallic layer, especially those described in documents JP-A-09188830, JP-A-10158450, JP-A-10158541, JP-A-07258460 and JP-A-05017710.

Hydrophobic Treatment of the Dyestuffs

The pulverulent dyestuffs as described previously may be totally or partially surface-treated, with a hydrophobic agent, to make them more compatible with the oily phase of the composition of the invention, especially so that they have good wettability with oils. Thus, these treated pigments are well dispersed in the oily phase.

Hydrophobic-treated pigments are described especially in document EP-A-1 086 683.

The hydrophobic-treatment agent may be chosen from silicones such as methicones, dimethicones and perfluoroalkylsilanes; fatty acids, such as stearic acid; metal soaps, such as aluminum dimyristate, the aluminum salt of hydrogenated tallow glutamate; perfluoroalkyl phosphates, polyhexafluoropropylene oxides; perfluoropolyethers; amino acids; N-acylamino acids or salts thereof; lecithin, isopropyl triisostearyl titanate, isostearyl sebacate, and mixtures thereof.

The term “alkyl” mentioned in the compounds cited above especially denotes an alkyl group containing from 1 to 30 carbon atoms and preferably containing from 5 to 16 carbon atoms.

Advantageously, a composition according to the invention may also comprise one or more fillers conventionally used in care and/or makeup compositions.

These fillers are colorless or white solid particles of any form, which are in a form that is insoluble and dispersed in the medium of the composition.

These fillers, of mineral or organic, natural or synthetic nature, give the composition containing them softness and give the makeup result a matt effect and uniformity. In addition, these fillers advantageously make it possible to combat various attacking factors such as sebum or sweat.

As illustrations of these fillers, mention may be made of talc, mica, silica, kaolin, poly-β-alanine powder and polyethylene powder, powders of tetrafluoroethylene polymers (Teflon®), lauroyllysine, starch, boron nitride, hollow polymer microspheres such as those of polyvinylidene chloride/acrylonitrile, for instance Expancel® (Nobel Industrie), acrylic acid copolymer microspheres, silicone resin microbeads (for example Tospearls® from Toshiba), polyorganosiloxane elastomer particles, precipitated calcium carbonate, magnesium carbonate, magnesium hydrogen carbonate, hydroxyapatite, barium sulfate, aluminum oxides, polyurethane powders, composite fillers, hollow silica microspheres, and glass or ceramic microcapsules. Use may also be made of particles which are in the form of hollow sphere portions, as described in patent applications JP-2003 128 788 and JP-2000 191 789.

In particular, such fillers may be present in a composition according to the invention in a content of between 0.01% and 30% by weight, especially between 0.1% and 25% by weight and in particular between 1% and 20% by weight relative to the total weight of the composition.

According to one embodiment of the invention, a composition may comprise at least solid particles such as pigments and/or fillers.

Dispersant

Advantageously, a composition according to the invention may also comprise a dispersant.

Such a dispersant may be a surfactant, an oligomer, a polymer or a mixture of several thereof.

According to a particular embodiment, a dispersant in accordance with the invention is a surfactant.

According to a particular variant, a composition according to the invention comprises at least 1% by weight of surfactant relative to the total weight of the composition, or even is free of surfactant.

Active Agent

For a particular care application, a composition according to the invention may comprise at least one moisturizer (also known as a humectant).

Preferably, the moisturizer is glycerol.

The moisturizer(s) may be present in the composition in a content ranging from 0.1% to 30% by weight, especially from 0.5% to 15% by weight or even from 1% to 10% by weight relative to the total weight of said composition.

As other active agents that may be used in the composition of the invention, mention may be made, for example, of vitamins.

Preferably, a composition according to the invention comprises at least one active agent.

It is a matter of routine operations for those skilled in the art to adjust the nature and the amount of the additives present in the compositions in accordance with the invention such that the desired cosmetic properties thereof are not thereby affected.

According to one embodiment, a composition of the invention may advantageously be in the form of a photoprotective composition for caring for the skin and/or keratin fibers, especially the hair, in particular of the body or the face.

According to another embodiment, a composition of the invention may advantageously be in the form of a makeup base composition.

According to another embodiment, a composition of the invention may advantageously be in the form of a foundation.

According to one embodiment, a composition of the invention may advantageously be in the form of a composition for making up the skin and especially the face. It may thus be an eyeshadow or a face powder. According to another embodiment, a composition of the invention may advantageously be in the form of a lip product, in particular a lipstick.

Such compositions are in particular prepared according to the general knowledge of those skilled in the art.

Throughout the description, including the claims, the term “comprising a” should be understood as being synonymous with “comprising at least one”, unless otherwise specified.

The terms “between . . . and . . . ” and “ranging from . . . to . . . ” should be understood as being inclusive of the limits, unless otherwise specified.

The invention is illustrated in greater detail by the examples presented below. Unless otherwise indicated, the amounts shown are expressed as percentages by weight.

Methodology for the Oscillating dynamic Rheology Measurements

These are harmonic-regime rheology measurements for measuring the elastic modulus.

The measurements are taken using a Haake RS600 rheometer on a product at rest, at 25° C. with a plate-plate rotor Ø60 mm and a 2 mm gap.

The harmonic-regime measurements make it possible to characterize the viscoelastic properties of the products. The technique consists in subjecting a material to a stress which varies sinusoidally over time and in measuring the response of the material to this stress. In a range in which the behavior is linear viscoelastic behavior (zone in which the strain is proportional to the stress), the stress (τ) and the strain (γ) are two sinusoidal functions of time which are written in the following manner:

τ(t)=τ₀ sin(ωt)

γ(t)=γ₀ sin(ωt+δ)

in which:

τ₀ represents the maximum amplitude of the stress (Pa);

γ₀ represents the maximum amplitude of the strain (−);

ω=2πN represents the angular frequency (rad.s⁻¹) with N representing the frequency (Hz); and

δ represents the phase shift of the stress relative to the strain (rad).

Thus, the two functions have the same angular frequency, but they are shifted by an angle δ. Depending on the phase shift δ between τ(t) and γ(t), the behavior of the system may be apprehended:

if δ=0, the material is purely elastic;
if δ=π/2, the material is purely viscous (Newtonian fluid); and
if 0<δ<π/2, the material is viscoelastic.

In general, the stress and the strain are written in complex form:

τ*(t)=τ₀ e^{iωt}

γ*(t)=γ₀ e^{(iωt+δ)}

A complex stiffness modulus, representing the overall resistance of the material to the strain, whether it is of elastic or viscous origin, is then defined by:

G*=τ*/γ*=G′+iG″

in which:

G′ is the storage modulus or elastic modulus, which characterizes the energy stored and totally restituted during a cycle, G′=(τ₀/γ₀) cos δ; and

G″ is the loss modulus or viscous modulus, which characterizes the energy dissipated by internal friction during a cycle, G″=(τ₀/γ₀) sin δ.

The parameter retained is the mean stiffness modulus G* recorded at the plateau measured at a frequency of 1 Hz.

### EXAMPLES

In the tables that follow, the amount of each compound is given as a weight percentage of raw material relative to the total weight of the composition.

The following formulations are prepared such that the following remain constant:
the mass percentage of the oily phase,
the mass percentage of the aqueous phase,
the mass percentage of oily gelling agent,
the mass percentage of aqueous gelling agent,
the mass percentage of UV-screening agents.

All the other constituents of the formulations are present in the same mass percentage.

A/ First Series of Examples

Preparation of the Compositions

Preparation of the Lipophilic Phases L

The fatty phase is gelled with at least one oily gelling agent with or without organic or particulate screening agents.

In the compositions according to the invention, the fatty phase is gelled with Disteardimonium hectorite (Bentone 38 V) alone (Examples 1-2 and 9).

The comparative examples are, themselves, gelled either with ethylcellulose (Examples 3, 4, 10, 11 and 12) or with a high-melting apolar hydrocarbon-based wax, microcrystalline wax (Examples 5 and 6) or a silicone polyamide (Examples 7 and 8).

Procedure: In a first stage, all of the liposoluble screening agents and of the hot-soluble starting materials are weighed out in a beaker and dissolved with mechanical stirring at 80° C.

As soon as the solution of screening agents is macroscopically clear, the oily gelling agents are added with mechanical stirring using a “deflocculator”. As soon as the homogeneous gelled phase is obtained, the solvents are added with the same mechanical stirring. The gel obtained constitutes a homogeneous gel.

Preparation of the Hydrophilic Gels H

The components of the aqueous phase are weighed out in a beaker and stirred.

The aqueous phase is gelled with at least one aqueous gelling agent with or without organic or particulate screening agents.

In the compositions according to the invention, the aqueous phase is gelled with hydroxyethyl acrylate/sodium acryloydimethyl taurate copolymer—Sepinov EMT 10 (Examples 1 and 2) or with a Carbomer (Example 9).

The comparative examples are gelled either with hydroxyethyl acrylate/sodium acryloydimethyl taurate copolymer—Sepinov EMT 10 (comparative Examples 3 to 8) or with a Carbomer (Example 10 outside the invention) or with a poloxamer (Example 11 outside the invention) or sodium CMC (Example 12 outside the invention).

Procedure: The aqueous gelling agent is introduced into the aqueous solvents with stirring using a “deflocculator” at room temperature. The gel obtained constitutes a homogeneous gel.

Gel/Gel Procedure

Since the lipophilic and hydrophilic phases are homogeneous, the gel/gel is prepared by mixing the two phases in a “kneader”-type mixer equipped with a tank and an axial paddle with moderate stirring for 4 minutes.

The final gel is characterized by a macroscopically homogeneous bicontinuous dispersion.

Properties Tested

Observation of the macroscopic appearance of the oily gel, of the aqueous gel and of the gel/gel is observed at:

t0, i.e. at the end of formulation, on exiting the tank;

t1, i.e. after two hours of leaving to stand at room temperature.

Compositions:

| | | | Example 2 | | |
|---|---|---|---|---|---|
| | Compounds | Example 1 | In | Example 3 | Example 4 |
| | INCI name | In accordance | accordance | Comparative | Comparative |
| LIPOPHILIC | Ethylhexyl salicylate | 5 | 5 | 5 | 5 |
| PHASE B | sold under the name Neo | | | | |
| | Heliopan OS by Symrise | | | | |
| | Octocrylene sold under the | 7 | 7 | 7 | 7 |
| | name Uvinul N539 T by | | | | |
| | BASF | | | | |
| | Butyl | 3 | 3 | 3 | 3 |
| | methoxydibenzoylmethane | | | | |
| | sold under the name | | | | |
| | Avobenzone by MFCI | | | | |
| | Disteardimonium hectorite | 5 | 5 | — | — |
| | sold under the name | | | | |
| | Bentone 38 VCG by | | | | |
| | Elementis | | | | |
| | Ethylcellulose sold under | — | — | 5 | 5 |
| | the name Ethocel by Dow | | | | |
| | Chemicals | | | | |
| | Propylene carbonate sold | 1.5 | 1.5 | 1.5 | 1.5 |
| | under the name Arconate | | | | |
| | propylene carbonate by | | | | |
| | Lyondell | | | | |
| | Octyldodecanol sold under | 27.7 | 27.7 | 27.7 | 27.7 |
| | the name Eutanol G by | | | | |
| | Cognis | | | | |
| | Caprylyl glycol sold under | 0.1 | 0.1 | 0.1 | 0.1 |
| | the name Dermasoft Octiol | | | | |
| | by Dr Straetmans | | | | |
| | Phenoxyethanol sold under | 0.5 | 0.5 | 0.5 | 0.5 |
| | the name Sepicide LD by | | | | |
| | SEPPIC | | | | |
| | Disodium EDTA sold | 0.2 | 0.2 | 0.2 | 0.2 |
| | under the name EDETA | | | | |
| | BD by BASF | | | | |
| HYDROPHILIC | Terephthalylidenedi- | — | 18 | — | 18 |
| PHASE A | camphorsulfonic acid (at | | | | |
| | 33% AM) sold under the | | | | |
| | name Mexoryl SX by | | | | |
| | Chimex | | | | |
| | Phenylbenzimidazole | — | 6 | — | 6 |
| | sulfonic acid sold under | | | | |
| | the name Eusolex 232 by | | | | |
| | Merck | | | | |
| | Triethanolamine sold | — | 6.75 | — | 6.75 |
| | under the name | | | | |
| | Triethanolamine by BASF | | | | |
| | Deionized water | 39.9 | 9.15 | 39.9 | 9.15 |
| | Caprylyl glycol sold under | 0.1 | 0.1 | 0.1 | 0.1 |
| | the name Dermasoft Octiol | | | | |
| | by Dr Straetmans | | | | |
| | Phenoxyethanol sold under | 0.5 | 0.5 | 0.5 | 0.5 |
| | the name Sepicide LD by | | | | |
| | SEPPIC | | | | |
| | Glycerol sold under the | 7 | 7 | 7 | 7 |
| | name Glycerine USP by | | | | |
| | VVF | | | | |
| | Hydroxyethyl | 2.5 | 2.5 | 2.5 | 2.5 |
| | acrylate/sodium | | | | |
| | acryloydimethyl taurate | | | | |
| | copolymer sold under the | | | | |
| | name Sepinov EMT 10 by | | | | |
| | SEPPIC | | | | |

Results:

For compositions 1 and 2 in accordance, the oily gel, the aqueous gel and the final gel/gel are always homogeneous at t0 and at t1.

However,
as regards the comparative composition 3, the macroscopic appearance of the composition at t1 appears phase-separated and
as regards the comparative composition 4, release of the oily gel at t1 and phase separation of the composition at t0 are observed.

| | Compounds | Example 5 | Example 6 | Example 7 | Example 8 |
|---|---|---|---|---|---|
| | INCI name | Comparative | Comparative | Comparative | Comparative |
| LIPOPHILIC | Ethylhexyl salicylate | 5 | 5 | 5 | 5 |
| PHASE B | sold under the name Neo | | | | |
| | Heliopan OS by Symrise | | | | |
| | Octocrylene sold under the | 7 | 7 | 7 | 7 |
| | name Uvinul N539 T by | | | | |
| | BASF | | | | |
| | Butyl | 3 | 3 | 3 | 3 |
| | methoxydibenzoylmethane | | | | |
| | sold under the name | | | | |
| | Avobenzone by MFCI | | | | |
| | Microcrystalline Wax sold | 5 | 5 | — | — |
| | under the name Microwax | | | | |
| | HW by Paramelt | | | | |
| | Nylon-611/dimethicone | — | — | 5 | 5 |
| | copolymer sold under the | | | | |
| | name Dow Corning 2-8179 | | | | |
| | Gellant by Dow Corning | | | | |
| | Propylene carbonate sold | 1.5 | 1.5 | 1.5 | 1.5 |
| | under the name Arconate | | | | |
| | propylene carbonate by | | | | |
| | Lyondell | | | | |
| | Octyldodecanol sold under | 27.7 | 27.7 | 27.7 | 27.7 |
| | the name Eutanol G by | | | | |
| | Cognis | | | | |
| | Caprylyl glycol sold under | 0.1 | 0.1 | 0.1 | 0.1 |
| | the name Dermasoft Octiol | | | | |
| | by Dr Straetmans | | | | |
| | Phenoxyethanol sold under | 0.5 | 0.5 | 0.5 | 0.5 |
| | the name Sepicide LD by | | | | |
| | SEPPIC | | | | |
| | Disodium EDTA sold | 0.2 | 0.2 | 0.2 | 0.2 |
| | under the name EDETA | | | | |
| | BD by BASF | | | | |
| HYDROPHILIC | Terephthalylidenedi- | — | 18 | — | 18 |
| PHASE A | camphorsulfonic acid (at | | | | |
| | 33% AM) sold under the | | | | |
| | name Mexoryl SX by | | | | |
| | Chimex | | | | |
| | Phenylbenzimidazole | — | 6 | — | 6 |
| | sulfonic acid sold under | | | | |
| | the name Eusolex 232 by | | | | |
| | Merck | | | | |
| | Triethanolamine sold | — | 6.75 | — | 6.75 |
| | under the name | | | | |
| | Triethanolamine by BASF | | | | |
| | Deionized water | 39.9 | 9.15 | 39.9 | 9.15 |
| | Caprylyl glycol sold under | 0.1 | 0.1 | 0.1 | 0.1 |
| | the name Dermasoft Octiol | | | | |
| | by Dr Straetmans | | | | |
| | Phenoxyethanol sold under | 0.5 | 0.5 | 0.5 | 0.5 |
| | the name Sepicide LD by | | | | |
| | SEPPIC | | | | |
| | Glycerol sold under the | 7 | 7 | 7 | 7 |
| | name Glycerine USP by | | | | |
| | VVF | | | | |
| | Hydroxyethyl | 2.5 | 2.5 | 2.5 | 2.5 |
| | acrylate/sodium | | | | |
| | acryloydimethyl taurate | | | | |
| | copolymer sold under the | | | | |
| | name Sepinov EMT 10 by | | | | |
| | SEPPIC | | | | |

Results:

For all the comparative compositions 5, 6, 7 and 8 during the study of the macroscopic appearance of the oily gel, a lump of said oily gel is observed from t0, accompanied for compositions 7 and 8 by the presence of surface oil.

In addition, phase separation of all of these comparative compositions is observed from t0.

| | Compounds | Example 9 | Example 10 | Example 11 | Example 12 |
|---|---|---|---|---|---|
| | INCI name | In accordance | Comparative | Comparative | Comparative |
| LIPOPHILIC | Ethylhexyl | 7 | 7 | 7 | 7 |
| PHASE B | methoxycinnamate sold | | | | |
| | under the name Parsol | | | | |
| | MCX by BASF | | | | |
| | Disteardimonium hectorite | 5 | — | — | — |
| | sold under the name | | | | |
| | Bentone 38 VCG by | | | | |
| | Elementis | | | | |
| | Ethylcellulose sold under | — | 5 | 5 | 5 |
| | the name Ethocel by Dow | | | | |
| | Chemicals | | | | |
| | Propylene carbonate sold | 1.5 | — | — | — |
| | under the name Arconate | | | | |
| | propylene carbonate by | | | | |
| | Lyondell | | | | |
| | Caprylic/Capric | 35.9 | 37.4 | 37.4 | 37.4 |
| | Triglyceride sold under the | | | | |
| | name Triglyceride C8/C10 | | | | |
| | by Stéarineries Dubois | | | | |
| | Caprylyl glycol sold under | 0.1 | 0.1 | 0.1 | 0.1 |
| | the name Dermasoft Octiol | | | | |
| | by Dr Straetmans | | | | |
| | Phenoxyethanol sold under | 0.5 | 0.5 | 0.5 | 0.5 |
| | the name Sepicide LD by | | | | |
| | SEPPIC | | | | |
| HYDROPHILIC | Deionized water | 46.9 | 46.9 | 46.9 | 46.9 |
| PHASE A | Caprylyl glycol sold under | 0.1 | 0.1 | 0.1 | 0.1 |
| | the name Dermasoft Octiol | | | | |
| | by Dr Straetmans | | | | |
| | Phenoxyethanol sold under | 0.5 | 0.5 | 0.5 | 0.5 |
| | the name Sepicide LD by | | | | |
| | SEPPIC | | | | |
| | Carbomer sold under the | 1.25 | 1.25 | — | — |
| | name Carbopol 980 | | | | |
| | Polymer by Ashland | | | | |
| | Triethanolamine sold | 1.25 | 1.25 | — | — |
| | under the name | | | | |
| | Triethanolamine by BASF | | | | |
| | Synperonic PE/L 64-LQ- | — | — | 2.5 | — |
| | (CQ) sold under the name | | | | |
| | Ploxamer by Croda | | | | |
| | Sodium | — | — | — | 2.5 |
| | carboxymethylcellulose | | | | |
| | sold under the name | | | | |
| | Blanose by Ashland | | | | |

Results

For composition 9 in accordance, the oily gel, the aqueous gel and the final gel/gel are always homogeneous at t1.

However, for the comparative compositions 10, 11 and 12, the macroscopic appearance of the composition at t1 appears phase-separated.

Only Examples 1, 2 and 9 in accordance with the invention lead to stable, homogeneous gel/gel compositions which also have a pleasant sensory aspect.

B Second Series of Examples (Comprising a Silicone Elastomer and a Silica Aerogel)

Compositions

| | | Example 1 | Example 2 |
|---|---|---|---|
| | | In accordance | Comparative |
| | Compounds | (Gel/gel composition) | (direct emulsion) |
| LIPOPHILIC | Homosalate | 4 | 4 |
| PHASE B | sold under the name Neo | | |
| | Heliopan HMS PBF by | | |
| | Symrise | | |
| | Ethylhexyl salicylate | 2 | 2 |
| | sold under the name Neo | | |
| | Heliopan OS by Symrise | | |
| | Octocrylene sold under the | 5 | 5 |
| | name Uvinul N539 T by | | |
| | BASF | | |
| | Butyl | 4 | 4 |
| | methoxydibenzoylmethane | | |
| | sold under the name | | |
| | Avobenzone by MFCI | | |
| | Bis-ethylhexyloxyphenol | 2 | 2 |
| | methoxyphenyl triazine | | |
| | sold under the name | | |
| | Tinosorb S by BASF | | |
| | Ethylhexyl triazone sold | 1 | 1 |
| | under the name Uvinul | | |
| | T150 by BASF | | |
| | Disteardimonium hectorite | 2.69 | 2.69 |
| | sold under the name | | |
| | Bentone 38 VCG by | | |
| | Elementis | | |
| | Silyl silica sold under the | 1 | 1 |
| | name DC V %-2270 | | |
| | Aerogel Fine Particles by | | |
| | Dow Corning | | |
| | Dimethicone sold under | 7.1 | 7.1 |
| | the name DC Toray SH200 | | |
| | C Fluid 5cs by Dow | | |
| | Corning | | |
| | Dimethicone and | 15.4 | 15.4 |
| | dimethicone crosspolymer | (i.e. 2.4% Dimethicone | (i.e. 2.4% Dimethicone |
| | sold under the name Dow | Crosspolymer) | Crosspolymer) |
| | Corning 9041 Silicone | | |
| | Elastomer Blend by Dow | | |
| | Corning | | |
| | Stearic acid sold under the | — | 1.5 |
| | name Radiacid 0461 by | | |
| | Oleon | | |
| | Glyceryl Stearate and | — | 1.5 |
| | PEG-100 stearate sold | | |
| | under the name Simulsol | | |
| | 165 by SEPPIC | | |
| | Denatured alcohol sold | 5.31 | 5.31 |
| | under the name Ethanol | | |
| | SDA 40B 200 Proof by | | |
| | Sasol | | |
| HYDROPHILIC | Deionized water | 38.35 | 38.35 |
| PHASE A | Disodium EDTA sold | 0.2 | 0.2 |
| | under the name EDETA | | |
| | BD by BASF | | |
| | Caprylyl glycol sold under | 0.7 | 0.7 |
| | the name Dermasoft Octiol | | |
| | by Dr Straetmans | | |
| | Phenoxyethanol sold under | 0.2 | 0.2 |
| | the name Sepicide LD by | | |
| | SEPPIC | | |
| | Glycerol sold under the | 6.6 | 6.6 |
| | name Glycerine USP by | | |
| | VVF | | |
| | Triethanolamine sold | — | 0.45 |
| | under the name | | |
| | Triethanolamine 99% by | | |
| | Dow Chemical | | |
| | Potassium cetyl phosphate | — | 1 |
| | sold under the name | | |
| | Amphisol K by DSM | | |
| | Nutritional Products | | |
| | Hydroxyethyl | 4.45 | — |
| | acrylate/sodium | | |
| | acryloydimethyl taurate | | |
| | copolymer sold under the | | |
| | name Sepinov EMT 10 by | | |
| | SEPPIC | | |

Preparation of the Compositions

Preparation of the Lipophilic Phases L

The fatty phase is gelled with at least the aerogel, the oily gelling agent with or without organic or particulate screening agents.

In Example 1, the fatty phase is gelled with aerogel combined with Bentone 38 VCG. Example 2 outside the invention is also gelled with the same gelling agents, but it contains in addition surfactants to form the emulsion.

In a first stage, all of the lipophilic screening agents and of the hot-soluble raw materials are weighed out in a beaker and dissolved with mechanical stirring at 80° C.

As soon as the solution of screening agents is macroscopically clear, the oily gelling agents are added with mechanical stirring using a “deflocculator”. Once a homogeneous gelled phase is obtained, the solvents are added with the same mechanical stirring. The gel obtained is homogeneous.

Preparation of the Hydrophilic Phases H

The components of the aqueous phase are weighed out in a beaker and stirred.

The aqueous phase is gelled with at least one aqueous gelling agent with or without organic or particulate screening agents.

In Example 1, the aqueous phase is gelled with Sepinov EMT 10.

The aqueous gelling agent is introduced into the aqueous solvents with stirring using a “deflocculator” at room temperature. The gel obtained is homogeneous.

The aqueous phase of Example 2 outside the invention is not gelled with Sepinov EMT 10, but contains an equivalent mass percentage of surfactants and combined neutralizer.

Gel/Gel Procedure Example 1 in Accordance

The gel/gel is prepared by mixing the two phases in a “kneader”-type mixer equipped with a tank and an axial paddle with moderate stirring for 4 minutes.

The final gel is characterized by a macroscopically homogeneous bicontinuous dispersion.

Emulsion Preparation Method—Comparative Example 2

The emulsion is prepared by introduction of phase L into phase H with stirring using a rotor-stator homogenizer at a stirring speed of 4500 rpm for 20 minutes. The emulsion is cooled to room temperature.

The final emulsion is characterized by drops between 1 μm and 20 μm in size.

Measurements

The SPF, the PPD and the sensory analysis of these compositions 1 and 2 were measured.

Protocol for Evaluating In Vitro the Screening Efficiency (SPF)

The sun protection factor (SPF) is determined according to the “in vitro” method described by B. L. Diffey in J. Soc. Cosmet. Chem. 40, 127-133, (1989). The measurements were made using a UV-1000S spectrophotometer from the company Labsphere. Each composition is applied to a rough plate of PMMA, in the form of a homogeneous and even deposit in a proportion of 1 mg/cm².

Protocol for Measuring the PPD In Vitro

The in vitro PPD index measurements were taken under the same conditions using a UV-1000S spectrophotometer from the company Labsphere. Each composition is applied to a rough plate of PMMA, in the form of a homogeneous and even deposit in a proportion of 1 mg/cm².

The UV-A ppd index: “Persistent Pigment Darkening” action spectrum value is extracted.

The UVA PPD sun protection factor (UVAppd PF) is expressed mathematically by the ratio of the dose of UVA radiation necessary to reach the pigmentation threshold with the UV-screening agent (MPPDp) to the dose of UVA radiation necessary to reach the pigmentation threshold without UV-screening agent (MPPDnp).

$${PFUVA}_{PPD} = \frac{MPPDp}{MPPDnp}$$

Protocol for Evaluating the Sensory Properties of the Formulations on the Skin

The sensory properties of the formulations on the skin are evaluated by applying the formulation to a forearm in a proportion of 2 mg/cm² and allowing a drying time equal to 2 minutes. The freshness is evaluated during the application, whereas the greasy and soft aspects are assessed after application, between the fingers and the surface of the forearm.

Results

Results

| | Example 1 | Example 2 |
|---|---|---|
| Property tested | In accordance | Comparative |
| in vitro SPF | 43.9 ± 4.8 | 21.3 ± 3.9 |
| in vitro PPD | 21.8 ± 2.1 | 10.4 ± 1.1 |
| Sensory analysis | Fresh on application, soft | Greasier feel |

These results show that composition 1 of the invention makes it possible to obtain a higher level of screening efficiency than composition 2 (direct emulsion), while at the same time showing an improved sensory aspect.

C Third Series of Examples (Comprising a Silicone Elastomer)

Non-Dyed Compositions

Preparation of the Compositions

Preparation of the Lipophilic Phases L

The fatty phase is gelled with the oily gelling agent.

Procedure

In a first stage, all of the lipophilic screening agents and of the hot-soluble raw materials are weighed out in a beaker and dissolved with mechanical stirring at 80° C.

As soon as the solution of screening agents is macroscopically clear, the oily gelling agents and the solvents are added with mechanical stirring using a “deflocculator”. The gel obtained is homogeneous.

Preparation of the Hydrophilic Phases H

The components of the aqueous phase are weighed out in a beaker and stirred.

The aqueous phase is gelled with at least one hydrophilic gelling agent.

Procedure

The hydrophilic gelling agent is introduced into the aqueous solvents with stirring using a “deflocculator” at room temperature. The gel obtained is homogeneous.

Gel/Gel Procedure

The gel/gel is prepared by mixing the two phases in a “kneader”-type mixer equipped with a tank and an axial paddle with moderate stirring for 4 minutes.

The final gel is characterized by a macroscopically homogeneous bicontinuous dispersion.

Measurements

The protocol for in vitro evaluation of the screening efficiency (SPF), the protocol for measuring the PPD in vitro and the protocol for evaluating the sensory properties of the formulations on the skin correspond to those described for series B above.

Evaluation of the Haze Effect of the Formulations in a Thin Film

Compositions 1 and 2 were spread to a thickness of 50 microns on a transparent film and the soft-focus effect of each composition was evaluated via a “Haze” measurement.

The “Haze” corresponds to the percentage of light scattered relative to the total transmittance according to standard ASTM D 1003 (Standard Test Method for Haze and Luminous Transmittance of Transparent Plastics).

Protocol for Evaluating the Homogeneity of the Film

The formulation is spread in the form of a 30 μm film onto a glass plate using a manual film spreader. The homogeneity of the formulation film is then evaluated by observation of the deposits by eye.

The notes applied are as follows:

“−” a nonuniform deposit, which is characterized by the presence of substantial holes visible to the naked eye;

“+” a homogeneous deposit;

“++” a very homogeneous deposit, which is characterized by a very small number of visible holes.

| | | Example 1 | Example 2 |
|---|---|---|---|
| | Compounds | In accordance | In accordance |
| LIPO- | Homosalate | 4 | 4 |
| PHILIC | sold under the name Neo | | |
| PHASE B | Heliopan HMS PBF by | | |
| | Symrise | | |
| | Ethylhexyl salicylate | 2 | 2 |
| | sold under the name Neo | | |
| | Heliopan OS by Symrise | | |
| | Octocrylene sold under the | 7 | 7 |
| | name Uvinul N539 T by | | |
| | BASF | | |
| | Butyl | 3 | 3 |
| | methoxydibenzoylmethane | | |
| | sold under the name | | |
| | Avobenzone by MFCI | | |
| | Silyl silica sold under the | 1 | 1.13 |
| | name DC V %-2270 | | |
| | Aerogel Fine Particles by | | |
| | Dow Corning | | |
| | Disteardimonium hectorite | 2.69 | — |
| | sold under the name | | |
| | Bentone 38 VCG by | | |
| | Elementis | | |
| | Propylene carbonate sold | 0.81 | — |
| | under the name Arconate | | |
| | propylene carbonate by | | |
| | Lyondell | | |
| | Dimethicone sold under | 8.8 | 1 |
| | the name DC Toray SH200 | | |
| | C Fluid 5cs by Dow | | |
| | Corning | | |
| | Dimethicone and | 15.4 | — |
| | dimethicone crosspolymer | (i.e. 2.4% | |
| | sold under the name Dow | Dimethicone | |
| | Corning 9041 Silicone | Cross- | |
| | Elastomer Blend by Dow | polymer) | |
| | Corning | | |
| | Dimethicone and | — | 15.4 |
| | Dimethicone crosspolymer | | (i.e. 2.0% |
| | sold under the name EL- | | Dimethicone |
| | 9240 Silicone Elastomer | | Cross- |
| | Blend by Dow Corning | | polymer) |
| | Denatured alcohol sold | 4.5 | — |
| | under the name Ethanol | | |
| | SDA 40B 200 Proof by | | |
| | Sasol | | |
| HYDRO- | Deionized water | qs 100 | qs 100 |
| PHILIC | Preserving agent | 0.98 | 0.9 |
| PHASE A | Glycerol sold under the | 6.6 | 6.6 |
| | name Glycerine USP by | | |
| | VVF | | |
| | Hydroxyethyl | 2.25 | 2.25 |
| | acrylate/sodium | | |
| | acryloydimethyl taurate | | |
| | copolymer sold under the | | |
| | name Sepinov EMT 10 by | | |
| | SEPPIC | | |

Results

| | Example 1 |
|---|---|
| Property tested | In accordance |
| in vitro SPF | 46 ± 8 |
| in vitro PPD | 20 ± 3 |
| Haze on 50 μm films | 87.7 |

Composition 1 has good photoprotective properties (level of screening efficiency) and also substantial haze.

In addition, composition 1 has freshness and velvety properties and also very satisfactory sensory and matt-effect properties.

For composition 2, only the homogeneity property of the film was tested.

| Property tested | Example 2 |
|---|---|
| Homogeneity of the film (30 | ++ |
| μm), covering power | |

Composition 2 has very good homogeneity, which is reflected by a smooth appearance of the composition deposited, which is visible to the naked eye.

Tinted Compositions (Examples 3 to 9)

Foundation compositions are prepared according to the protocol below in accordance with the protocol for evaluating the homogeneity of the film, described above.

Preparation of the Compositions

Preparation of the Hydrophilic Phase H

The components of the aqueous phase are weighed out in a beaker and stirred with a Rayneri blender, at room temperature.

The aqueous gelling agent is added with stirring at room temperature. The stirring is adjusted so as not to incorporate air into the mixture. The mixture is stirred moderately for about 10 minutes at room temperature.

A homogeneous aqueous gel is obtained.

Preparation of the Lipophilic Phase L

The pigments are ground with the solvents of the lipophilic phase B using a three-roll mill. The ground material is then introduced into a beaker and stirred using a Rayneri blender at room temperature. The gelling agent is added with vigorous stirring at room temperature. The gel slowly thickens. The mixture is stirred vigorously for 10 minutes and the fatty phase is gelled with the oily gelling agent.

A homogeneous oily gel is obtained.

Preparation of the Foundation Formulation

The formulation is obtained by mixing the phases intended to form the foundation in accordance with the invention.

The aqueous and oily gels are weighed out and then mixed using a Rayneri blender with moderate stirring. The formulation is prepared from the weight proportions described in the formulations.

For the tinted compositions, only the homogeneity property of the film was tested.

| | | Example 3 | Example 4 | Example 5 |
|---|---|---|---|---|
| | Compounds | In accordance | In accordance | In accordance |
| LIPOPHILIC | Homosalate | 4 | 4 | 4 |
| PHASE B | sold under the name | | | |
| | Neo Heliopan HMS | | | |
| | PBF by Symrise | | | |
| | Ethylhexyl salicylate | 2 | 2 | 2 |
| | sold under the name | | | |
| | Neo Heliopan OS by | | | |
| | Symrise | | | |
| | Octocrylene sold under | 7 | 7 | 7 |
| | the name Uvinul N539 | | | |
| | T by BASF | | | |
| | Iron oxides coated with | 2.54 | 2.54 | 2.54 |
| | aluminum stearoyl | | | |
| | glutamate (NAI-C33- | | | |
| | 9001-10 sold by the | | | |
| | company Miyoshi | | | |
| | Kasei) | | | |
| | Titanium dioxide | 8.46 | 8.46 | 8.46 |
| | coated with aluminum | | | |
| | stearoyl glutamate | | | |
| | (NAI-TAO-77891 sold | | | |
| | by the company | | | |
| | Miyoshi Kasei) | | | |
| | Disteardimonium | — | 3 | — |
| | hectorite sold under the | | | |
| | name Bentone 38 VCG | | | |
| | by Elementis | | | |
| | Silyl silica sold under | — | — | 0.96 |
| | the name DC V %- | | | |
| | 2270 Aerogel Fine | | | |
| | Particles by Dow | | | |
| | Corning | | | |
| | Dimethicone and | 7 | 7 | 7 |
| | Dimethicone | (i.e. 0.9% | (i.e. 0.9% | (i.e. 0.9% |
| | crosspolymer sold | Dimethicone | Dimethicone | Dimethicone |
| | under the name EL- | Crosspolymer) | Crosspolymer) | Crosspolymer) |
| | 9240 Silicone | | | |
| | Elastomer Blend by | | | |
| | Dow Corning | | | |
| HYDROPHILIC | Deionized water | qs 100 | qs 100 | qs 100 |
| PHASE A | Preserving agent | 0.7 | 0.7 | 0.7 |
| | Glycerol sold under the | 6 | 6 | 6 |
| | name Glycerine USP | | | |
| | by VVF | | | |
| | Hydroxyethyl | 2.4 | 2.4 | 2.4 |
| | acrylate/sodium | | | |
| | acryloydimethyl taurate | | | |
| | copolymer sold under | | | |
| | the name Sepinov EMT | | | |
| | 10 by SEPPIC | | | |

Results

| | Example 3 | Example 4 | Example 5 |
|---|---|---|---|
| | In accordance | In accordance | In accordance |
| Homogeneity of | + | ++ | ++ |
| the film (30 | | | |
| μm), covering | | | |
| power | | | |

| | | Example 6 | Example 7 | Example 8 | Example 9 |
|---|---|---|---|---|---|
| | | In | In | In | In |
| | Compounds | accordance | accordance | accordance | accordance |
| LIPOPHILIC | Homosalate | 4 | 4 | 4 | 4 |
| PHASE B | sold under the name Neo | | | | |
| | Heliopan HMS PBF by | | | | |
| | Symrise | | | | |
| | Ethylhexyl salicylate | 2 | 2 | 2 | 2 |
| | sold under the name Neo | | | | |
| | Heliopan OS by Symrise | | | | |
| | Octocrylene sold under the | 7 | 7 | 7 | 7 |
| | name Uvinul N539 T by | | | | |
| | BASF | | | | |
| | Iron oxides coated with | 2.54 | 2.54 | 2.54 | 2.54 |
| | aluminum stearoyl | | | | |
| | glutamate (NAI-C33-9001- | | | | |
| | 10 sold by the company | | | | |
| | Miyoshi Kasei) | | | | |
| | Titanium dioxide coated | 8.46 | 8.46 | 8.46 | 8.46 |
| | with aluminum stearoyl | | | | |
| | glutamate (NAI-TAO-77891 | | | | |
| | sold by the company | | | | |
| | Miyoshi Kasei) | | | | |
| | Dimethicone and | 7 | — | — | — |
| | dimethicone crosspolymer | (i.e. 1.1% | | | |
| | sold under the name Dow | Dimethicone | | | |
| | Corning 9041 Silicone | Crosspolymer) | | | |
| | Elastomer Blend by Dow | | | | |
| | Corning | | | | |
| | Diphenylsiloxy phenyl | — | 7 | — | — |
| | trimethicone 84% | | | | |
| | dimethicone/phenyl vinyl | | | | |
| | dimethicone crosspolymer | | | | |
| | 16% (KSG 18A sold by the | | | | |
| | company Shin-Etsu) | | | | |
| | Vinyl | — | — | 7 | — |
| | dimethicone/methicone | | | | |
| | silsesquioxane crosspolymer | | | | |
| | (KSP 100 sold by the | | | | |
| | company Shin-Etsu) | | | | |
| | Diphenyl dimethicone/vinyl | — | — | — | 7 |
| | diphenyl | | | | |
| | dimethicone/silsesquioxane | | | | |
| | crosspolymer (KSP 300 sold | | | | |
| | by the company Shin-Etsu) | | | | |
| HYDROPHILIC | Deionized water | qs 100 | qs 100 | qs 100 | qs 100 |
| PHASE A | Preserving agent | 0.7 | 0.7 | 0.7 | 0.7 |
| | Glycerol sold under the | 6 | 6 | 6 | 6 |
| | name Glycerine USP by | | | | |
| | VVF | | | | |
| | Hydroxyethyl | 2.4 | 2.4 | 2.4 | 2.4 |
| | acrylate/sodium | | | | |
| | acryloydimethyl taurate | | | | |
| | copolymer sold under the | | | | |
| | name Sepinov EMT 10 by | | | | |
| | SEPPIC | | | | |

Results

| | Example 6 | Example 7 | Example 8 | Example 9 |
|---|---|---|---|---|
| | In accor- | In accor- | In accor- | In accor- |
| | dance | dance | dance | dance |
| Homogeneity | ++ | ++ | ++ | ++ |
| of the film | | | | |
| (30 μm), | | | | |
| covering power | | | | |

Compositions 3 to 9 have very good homogeneity, which is reflected by a smooth appearance of the composition deposited, this property being visible to the naked eye.

1. Composition for making up and/or caring for keratin materials, comprising:
at least one aqueous phase gelled with at least one non-starchy hydrophilic gelling agent,
at least one oily phase gelled with at least one non-cellulose-based lipophilic gelling agent other than apolar hydrocarbon-based waxes with a melting point of greater than 75.0° C. and silicone polyamides;
said phases forming therein a macroscopically homogeneous mixture;
said composition also comprising at least one UV-screening agent.

2. Composition according to claim 1, in which said non-starchy hydrophilic gelling agent is chosen from synthetic polymeric gelling agents, mixed silicates and fumed silicas, non-starchy polymeric gelling agents that are natural or of natural origin, and mixtures thereof.

3. Composition according to claim 2, in which the hydrophilic gelling agent is a synthetic polymeric gelling agent.

4. Composition according to claim 3, in which the synthetic polymeric hydrophilic gelling agent is chosen from crosslinked ammonium acrylamido-2-methylpropanesulfonate polymers, copolymers of AMPS° and of hydroxyethyl acrylate, and crosslinked (meth)acrylic acid homopolymers.

5. Composition according to claim 1, in which the lipophilic gelling agent is chosen from organopolysiloxane elastomers, semicrystalline polymers, dextrin esters, hydrocarbon-based polyamides, block hydrocarbon-based copolymers, particulate gelling agents chosen from polar waxes, hydrocarbon-based apolar waxes with a melting point of less than or equal to 75.0° C., silicone waxes, modified clays, silicas, and also mixtures thereof.

6. Composition according to claim 5, in which the lipophilic gelling agent is chosen from semicrystalline homopolymers or copolymers bearing at least one crystallizable side chain and semicrystalline homopolymers or copolymers bearing at least one crystallizable block in the backbone, hydrocarbon-based polyamides, hydrophobic silica aerogels, hectorites modified with a C₁₀ to C₂₂ ammonium salt.

7. Composition according to claim 6, in which the lipophilic gelling agent is chosen from hectorites modified with a C₁₀ to C₂₂ ammonium salt.

8. Composition according to claim 5, in which the lipophilic gelling agent is chosen from the following organopolysiloxane elastomers:
dimethicone crosspolymer, vinyl dimethicone crosspolymer, dimethicone/vinyl dimethicone crosspolymer, dimethicone crosspolymer-3, vinyl dimethicone/methicone silsesquioxane crosspolymer, phenyl vinyl dimethicone crosspolymer.

9. Composition according to claim 1, comprising
at least one non-starchy hydrophilic gelling agent chosen from crosslinked and/or neutralized 2-acrylamido-2-methylpropanesulfonic acid (AMPS®) copolymers, and
at least one non-cellulose-based lipophilic gelling agent chosen from hectorites modified with a salt,
at least one UV-screening agent.

10. Composition according to claim 1, in which the UV-screening agent(s) are chosen from water-soluble organic UV-screening agents, liposoluble organic UV-screening agents, and mixtures thereof.

11. Composition according to claim 1, in which the UV-screening agent(s) are totally or partly present in the gelled aqueous phase or are totally or partly present in the gelled oily phase.

12. Composition according to claim 1, containing the gelled aqueous and oily phases in an aqueous phase/oily phase weight ratio of from 95/5 to 5/95.

13. Composition according to claim 1, also comprising at least solid particles.

14. Composition according to claim 1, also comprising volatile and/or non-volatile silicone oils.

15. Composition according to claim 1, also comprising at least one moisturizer.

16. Process for preparing a composition for making up and/or caring for keratin materials, comprising at least one step of mixing:
an aqueous phase gelled with at least one non-starchy hydrophilic gelling agent; and
at least one oily phase gelled with at least one non-cellulose-based lipophilic gelling agent other than apolar hydrocarbon-based waxes with a melting point of greater than 75.0° C. and silicone polyamides;
said phases forming therein a macroscopically homogeneous mixture;
said composition also comprising at least one UV-screening agent.

17. Process according to claim 16, comprising a step of mixing at least two gelled phases.

18. Process according to claim 16, in which the mixing is performed at room temperature.

19. Cosmetic process for making up and/or caring for keratin materials and/or keratin fibers, comprising at least one step which consists in applying to said keratin material a composition as defined according to claim 1.

20. Cosmetic process for making up and/or caring for keratin materials and/or keratin fibers, comprising at least the application to said keratin materials of a macroscopically homogeneous composition obtained by extemporaneous mixing, before application or at the time of application to said keratin material, of
an aqueous phase gelled with at least one non-starchy hydrophilic gelling agent; and
at least one oily phase gelled with at least one non-cellulose-based lipophilic gelling agent other than apolar hydrocarbon-based waxes with a melting point of greater than 75.0° C. and silicone polyamides; and
said composition also comprising at least one UV-screening agent.

21. Cosmetic process for limiting the darkening of the skin and/or improving the color and/or uniformity of the complexion, comprising the application, to the surface of the keratin material, of a composition as defined according to claim 1.

22. Cosmetic process for preventing and/or treating the signs of aging of a keratin material, comprising the application, to the surface of the keratin material, of a composition as defined according to claim 1.
